(19) United States
(12) Patent Application Publication
(10) Pub. No.: US 2022/0062354 A1
(43) Pub. Date: Mar. 3, 2022

(54) TOPICAL FORMULATIONS FOR THE DELIVERY OF MICROBIALLY DERIVED MATERIALS

(71) Applicant: Matrisys Bioscience, Inc., La Jolla, CA (US)

(72) Inventor: Louis H. Bookbinder, San Diego, CA (US)

(21) Appl. No.: 17/298,924

(22) PCT Filed: Dec. 18, 2019
(86) PCT No.: PCT/US2019/067061

§ 371 Date: Jun. 1, 2021

Related U.S. Application Data
(60) Provisional application No. 62/784,060, filed on Dec. 21, 2018.

(51) Int. Cl. A61K 35/741; A61K 47/44; A61K 9/06; A61K 9/00; A61P 31/04

(52) U.S. Cl. CPC A61K 35/741; A61K 47/44; A61P 31/04; A61K 9/0014; A61K 9/06

(57) ABSTRACT

The present disclosure describes topical formulations for the administration of probiotic skin bacteria and extracts or derivatives thereof. The present disclosure further describes methods and compositions for assuring the long-term storage and stability of said formulations.

### RELATED APPLICATIONS

This application claims the benefit under 35 U.S.C. §119(e) of U.S. Provisional Patent Application Ser. No. 62/784,060, filed Dec. 21, 2018, the content of this related application is incorporated herein by reference in its entirety for all purposes.

### BACKGROUND

### Field

The present disclosure relates generally to the field of treatments for skin disorders and more specifically to the use of microbes and microbial derived materials for said treatments.

### Description of the Related Art

Patients with atopic dermatitis (AD) have recurrent skin infections by Staphylococcus aureus (SA) and dysbiosis of their cutaneous microbiome. The increased susceptibility to SA has been associated with diminished innate immune defense including abnormal barrier function and decreased induction of antimicrobial peptides (AMPs) such as cathelicidin and β-defensins. Dysbiosis of the cutaneous microbiome allows establishment and/or overgrowth of SA and other potential skin pathogens and may contribute to irregularities in innate immunity. Correction of the underlying dysbiosis may therefore be an effective way to treat, ameliorate, or cure diseases or disorders associated with cutaneous dysbiosis, such as atopic dermatitis.

Symptoms of atopic dermatitis, also referred to as eczema or atopic eczema include: dry skin that forms a rash; scaly, swollen, and red skin; rash on the face, or inside the knees, elbows, or wrists; blisters that ooze; changes in skin color after repeated episodes; thickened, cracked, dry, scaly skin or skin that looks leathery in patches; and severe itchiness (pruritis), especially at night, along with raw, sensitive, swollen skin from scratching. Atopic dermatitis (eczema) signs and symptoms vary widely from person to person and may further include: red to brownish-gray patches, especially on the hands, feet, ankles, wrists, neck, upper chest, eyelids, inside the bend of the elbows and knees, and, in infants, the face, scalp, back of the head, ears, legs, feet, arms, hands and buttocks; small, raised bumps, which may leak fluid and crust over when scratched. Atopic dermatitis most often begins before age 5 and may persist into adolescence and adulthood. For some people, it flares periodically and then clears up for a time, even for several years. The skin changes brought about by atopic dermatitis can facilitate high susceptibility of these subjects to colonization and infections by Staphylococcus aureus, Staphylococcus schleiferi, Staphylococcus intermedius, Staphylococcus pseudintermedius, Staphylococcus felis, or other bacterial infections such as Mallassezia, especially Mallassezia sympodialis, Mallassezia globosa, Micrococcus spp., Acinetobacter spp., alpha-hemolytic streptococci, and/or other pathogens of the skin or external mucosa.

Dysbiosis comprises an imbalance in the cutaneous or mucosal flora, including the nasal, oral, ophthalmic, urogenital, intestinal flora, wherein species such as S. aureus become overrepresented and other species become underrepresented. Generally, in a healthy flora, nonpathogenic bacteria may secrete inhibitors or simply occupy all available niches, thus either directly inhibiting or indirectly excluding pathogens that would otherwise be able to establish infectious states or foster the development of disease or disease-like states, such as atopic dermatitis. Further, treatment of bacterial overgrowth or infection with either topical or systemic antibiotics may lead to further dysbiosis as beneficial and/or health associated bacteria are killed or restricted from growing. Nonspecific bactericidal treatments such as hypochlorite or triclosan rinses may yield similar effects. Importantly, the skin comprises a large surface area with numerous involutions, cracks, pores, hair follicles, and other microenvironments, many of which serve to protect bacteria living therein form insult or attack by systemically or topically applied antimicrobial or anti-infective compounds. Restoration of the normal skin or mucosal microbiota could have the effect of leveraging the biology of commensal organisms to exclude pathogens from these niches, or to actively inhibit growth or colonization by disease-causing organisms.

Accordingly there is a need for the development and delivery of compositions that can restore the healthy skin flora, either by reducing the abundance of pathogenic bacteria or by increasing the abundance of health-associated bacteria, thereby treating, ameliorating or curing diseases or disorders associated with dysbiosis, such as atopic dermatitis.

### SUMMARY

In some embodiments, the present disclosure contemplates a composition comprising one or more probiotic bacteria and further comprising an oil and at least one pharmaceutically acceptable excipient. In some embodiments, the one or more probiotic bacteria may be or may comprise a bacterium of the genus Staphylococcus. In some embodiments, a bacterium of the genus Staphylococcus may be or may comprise one or more bacteria of the species Staphylococcus hominis and/or Staphylococcus epidermis. In some embodiments, one or more bacteria of the species Staphylococcus hominis and/or Staphylococcus epidermis may be or may comprise a strain of Staphylococcus hominis, and in particular, Staphylococcus hominis strain A9.

Disclosed herein include compositions. In some embodiments, the composition comprises an active drug substance comprising lyophilized coagulase negative Staphylococcus (CoNS); an inert particle; and an oil. In some embodiments, the composition comprises an active drug substance comprising lyophilized Staphylococcus hominis strain A9; fumed silica; and an oil selected from the group comprising soy oil, sesame oil, mineral oil, corn oil, olive oil, peanut oil, macadamia nut oil, canola oil, emu oil, or any combination thereof. In some embodiments, the compositions provided herein comprise a sugar, a starch, a cellulose, powdered tragacanth, malt, gelatin, talc, a solid lubricant, calcium sulfate, a vegetable oil, a polyol, alginic acid; a TWEEN, sodium lauryl sulfate, an emulsifier, a wetting agent, a coloring agent, a flavoring agent, a tableting agent, a stabilizer; an antioxidant, a preservative, pyrogen-free water, isotonic saline, a phosphate buffer solution, or any combination thereof.

In some embodiments, the compositions disclosed herein comprise an oil, which may be or may comprise one or more of soy oil, sesame oil, mineral oil, corn oil, olive oil, peanut oil, macadamia nut oil, canola oil, or emu oil. In some embodiments the oil is or comprises soy oil. In some embodiments the oil is or comprises sesame oil.

In some embodiments, the compositions disclosed herein comprise a pharmaceutically acceptable excipient, which may be or may comprise one or more of tocopherol, monosodium glutamate, starch, colloidal silicon dioxide, microcrystalline cellulose, alginate, magnesium stearate, sodium stearate, stearyl alcohol, acetyl alcohol, cetostearyl alcohol, vinyl alcohol, or polyvinyl alcohol.

In some embodiments, the compositions disclosed herein may be formulated as oil compositions. In some embodiments, the compositions disclosed herein may be formulated as lotion compositions.

In some embodiments, the compositions disclosed herein may comprise one or more active drug substances or active cosmetic substances, wherein said active drug substance or active cosmetic substance may comprise one or more probiotic bacteria, lyophilized bacteria, growth media, lyophilized growth media, bacterial extracts, or lyophilized bacterial extracts. in some embodiments, an active drug substance may be present as a particle, microparticle, or nanoparticle. in some embodiments, said particle, microparticle, or nanoparticle has an average diameter of between 1 nm and 1 mm, between 10 nm and 500 um, between 100 nm and 100 um, between 1 um and 250 um, between 10 um and 100 um, between 10 um and 50 um, or between 20 um and 30 um.

In some embodiments, the compositions described herein are marked or identified by their ability to retain activity, especially bacterial growth or colony forming ability, after extended storage at room temperature. In particular, in some embodiments, the compositions disclosed herein may be stable for 2 months or more at room temperature, for 4 months or more at room temperature, or for 6 months or more at room temperature. In some embodiments, the compositions disclosed herein may be stable for 2 months or more at 4° C., for 4 months or more at 4° C., or for 6 months or more at 4° C. In some embodiments, composition as disclosed herein may retain at least 50% of its colony forming activity for 2 months or more at room temperature, at least 70% of its colony forming activity for 2 months or more at room temperature, or at least 80% of its colony forming activity for 2 months or more at room temperature. In some embodiments, a composition as disclosed herein may retain at least 50% of its colony forming activity for 2 months or more at 4° C., at least 70% of its colony forming activity for 2 months or more at 4° C., or at least 80% of its colony forming activity for 2 months or more at 4° C.

In some embodiments, a composition as disclosed herein may be anhydrous. or substantially free of moisture or water. In some embodiments, a composition as disclosed herein may be substantially free of water or moisture.

The present disclosure also contemplates a method of treating one or more diseases, disorders, or conditions of the skin by administering to a subject in need thereof one or more compositions as disclosed herein. In some embodiments, such a disease, disorder, or condition may comprise one or more of atopic dermatitis, eczema, pyotraumatic dermatitis, pyoderma, superficial pyoderma, folliculitis, rosacea, Netherton syndrome, acne, wounds (including abrasions, radiation damage, and burns), psoriasis, mastitis, icthyosis, lichen formation, and sebhorreic dermatitis, or any combination thereof, or any symptom, cause, or sequela thereof. In some embodiments, the disease, disorder, or condition to be treated may comprise or may further comprise a bacterial infection, overgrowth, or dysbiosis. In some embodiments, a bacterial infection, overgrowth, or dysbiosis may comprise or may further comprise colonization, overgrowth, or infection with one or more of Staphylococcus aureus, Staphylococcus intermedius, Staphylococcus pseudintermedius, Staphylococcus felis, Staphylococcus schleiferi, Micrococcus spp., Acinetobacter spp., and/or alpha hemolytic streptococci, Cutibacterium acnes or any combination thereof.

### BRIEF DESCRIPTION OF THE DRAWINGS

FIG. 1 shows S. aureus growth inhibition in a turbidity based growth assay using USA/300 MRSA UCSD as the test strain. Sh-A9 growth in AF-TSB of increasing concentration secrete relatively greater antimicrobial activity into the conditioned medium compared to the same seed of Sh-A9 grown in standard 1× AF-TSB. USA300 MRSA OD600 was measured after overnight incubation at 30° C. in Sh-A9 conditioned media.

FIG. 2 shows CFU recovery (CFU/mL) over 3 months in drug substance samples FM 1, 2, and 3. Initial cryoprotectant mixtures were stored in Eppendorf tubes at −20° C. after lyophilization, and CFU recovery was determined after water addition. FM1 and FM3 were identified as having superior CFU recovery.

FIG. 3 shows a comparison of CFU recovery from the 3 cryoprotectant mixtures over time at room temperature (17° C.) storage compared to −20° C. storage for non-milled lyophilized Sh-A9 “cakes” over 6 months. −20° C. storage demonstrated stable CFU recovery from all three cryoprotectant mixtures while 17° C. storage demonstrated 1 log reduction of CFU recovery by 6 months. Freeze Media 1 1× comprises: 10 gm Sucrose/100 ml=10%; 5 gm AF-Soytone/100 ml=5%; 0.75 gm AF TSB/100 ml=0.75%. Freeze Media 2 1× comprises: 2.5 gm Monosodium Glutamate/100 ml=2.5%; 4.0 gm Ascorbic Acid=4%; 10 gm Sucrose/100 ml=10%. Freeze Media 3 1× comprises: 4 gm Monosodium Glutamate/100 ml=4%; 2 gm Dextran 500/100 ml=2%; 2 gm Sorbitol/100 ml=2%.

FIG. 4 shows recovery of CFUs from Sesame oil formulations stored at 17° C. over 6 months (1-180 days). The data shown demonstrate stable recovery of CFUs from Sesame oil formulations throughout the 6 month test period. Powdered Sh-A9 open to the atmosphere lost significant CFUs over the first 30 days and became “cakey” and difficult to handle and recovery evaluation was discontinued after 30 days.

FIG. 5 shows three-month stability data of dried milled powders utilizing different cryoprotectant formulations, as indicated. Lyophilized drug substance (Sh-A9) powders FM#3 and FM#4 were produced. The freeze-dried bulks cakes were broken into pieces and milled through a 20 mesh screen followed by a 60 mesh screen. Powdered Sh-A9 was stored at −20° C. with desiccant. Dried powders were assayed for CFU recovery for stability. Freeze Media 3 1× comprises: 4 gm Monosodium Glutamate/100 ml=4%; 2 gm Dextran 500/100 ml=2%. Freeze Media 4 1× comprises: 1 gm Monosodium Glutamate/100 ml=1%; 2.5 gm Dextran 64-76K/100 ml=2.5%; 0.8 gm Sucrose/100 ml=0.8%; 0.42 gm Histidine/100 ml=0.42%; 5 gm Mannitol/100 ml=5%; pH to 7.4.

FIG. 6 shows CFU recovery (±SEM) from lyophilized Sh-A9 formulated in Sesame Oil with fumed silica (FM#1) over a 6-month period at 17° C. 100 Mesh Powder at room temperature and humidity. MSBFM#1 comprises 10× sucrose, 5% AF-Soytone. Vialed in sesame oil at room temperature and triplicate samples per vial diluted at day 6 and beyond for CFU determination.

FIG. 7 shows a comparison of CFU recovery from lyophilized Sh-A9 samples FM#3 and FM#4 formulated in various different oils (as indicated) over a 6 month period. FM#3 formulated in soybean oil or olive oil showed unexpectedly superior stability relative to other cryoprotectant formulations or oil formulations.

FIG. 8 shows a comparison of CFU recovery from lyophilized Sh-A9 samples of FM#3 and formulated in various different oils (as indicated) over a 6 month period at 17° C. As in FIG. 7, FM#3 formulated in soybean oil or olive oil showed unexpectedly superior stability relative to other cryoprotectant formulations or oil formulations. Further, presence of 800 to 1000 ppm BHT (butylated hydroxytoluene) as an oil preservative had no effect on recoverable CFUs. USP Soy oil with no preservative (NP) was used as a control to assess BHT toxicity to Sh-A9 Drug Products. USP Soy oil with or without BHT demonstrated similar CFU recovery over 6 months.

FIG. 9 shows the variability of CFU recovery over a 6 month period at 17° C. in FM#3 formulations in various oils. FM#3 lyophilized powder in topical oil formulations demonstrates stable recoverable CFUs for up to 6 months with <0.5 log range in variability over the time period. Average n=8 (e.g., 8 vials each formulation) measurements (CFU/mL) over 6 months ±SEM presented with SEM over the time varying by ˜±0.2 log.

FIGS. 10A-10D show an expanded analysis using day 7 to day 90 CFU/mL recovery data (n=6 time points) suggesting that the data variability measured may be due to the variability of the extraction process for any given sample rather than to product formulation destabilizing effects. Relative flatness of the regression trend lines and sigmoid point-to-point trend lines in the data below support this interpretation.

FIGS. 11A-11B show the stability of lyophilized Sh-A9 under refrigeration or at room temperature in standard Cetaphil/glycerol formulations. Significant CFU loss is seen after 47 days of refrigeration, and colony forming activity is essentially eliminated after 14 days at room temperature. FIG. 11A depicts non-limiting exemplary data related to the stability of Sh-A9 washed Bacteria freshly prepared in 50% Cetaphil/50% glycerol and stored at 4° C. FIG. 11B depicts non-limiting exemplary data related to the stability of Sh-A9 washed bacteria freshly prepared in 50% Cetaphil/50% glycerol and stored at 4° C. then transferred to 17° C.

FIGS. 12A-12C show the stability (CFU recovery) of lyophilized Sh-A9 in soy oil at 17° C. (FIG. 12A), 30° C. (FIG. 12B), and 45° C. (FIG. 12C) in the absence of fumed silica.

FIG. 13 shows the stability (CFU recovery) of lyophilized Sh-A9 in soy oil at room temperature (17° C.) in the presence of 0.23 mg/mg fumed silica per mg Sh-A9 lyophile.

FIGS. 14A-14C show the stability (CFU recovery) of lyophilized Sh-A9 in soy oil at room temperature (17° C.) (FIG. 14A), 30° C. (FIG. 14B), and 45° C. (FIG. 14C) in the presence of 0.34 mg fumed silica per mg Sh-A9 lyophile. Stability is significantly enhanced by the addition of 0.34% fumed silica.

FIGS. 15A-15C shows the stability (CFU recovery) of lyophilized Sh-A9 in soy oil at room temperature (17° C.) (FIG. 15A), 30° C. (FIG. 15B), and 45° C. (FIG. 15C) in the presence of 0.45 mg fumed silica per mg Sh-A9 lyophile. Stability is further enhanced by the addition of 0.45 mg/mg fumed silica.

FIGS. 16A-16C show the stability (CFU recovery) of lyophilized Sh-A9 in soy oil at room temperature (17° C.) (FIG. 16A), 30° C. (FIG. 16B), and 45° C. (FIG. 16C) in the presence of 0.64 mg fumed silica per mg Sh-A9 lyophile. Stability is enhanced by the addition of 0.64 mg/mg fumed silica relative to 0.23 mg/mg fumed silica.

FIG. 17 shows the stability of lyophilized Sh-A9 at room temperature in anhydrous lotion formulations (as indicated) containing beeswax. Freeze-dried Sh-A9 powder in FM#3 formulated in safflower oil and beeswax lotion (Sh-A9 Lotion #1 containing 5% super-refined beeswax) demonstrated a 1 log decrease in extractable CFU after 13 days at 17° C.

FIGS. 18A-18B, show the stability of lyophilized Sh-A9 at room temperature in anhydrous lotion formulations (soy oil/fatty alcohol lotion #1 (FIG. 18A) and soy oil/fatty alcohol lotion #2 (FIG. 18B)) containing fatty alcohols as described in Example 8. Soy oil/fatty alcohol lotion #2 comprises 0.48 mg silica/mg DS in some embodiments. Formulation #2 at 45° C. liquefied and turned yellow and was not measured.

### DETAILED DESCRIPTION

Dysbiosis may be a result of, or may be characterized by, loss of a beneficial bacterium, loss of a commensal bacterium, overgrowth of an otherwise beneficial or communal bacterium, establishment or overgrowth of a deleterious or pathogenic bacterium, or by any mechanism that results in an unstable or maladapted flora or microbiota, or a state of the flora or microbiota that is disordered or renders the host susceptible to any disease, disorder, or pathogenic state.

Providing a beneficial bacterium to remedy dysbiosis and disorders associated with dysbiosis has historically been difficult. Significant problems have been found with engraftment of bacterial cultures into the host microbiota, as well as with the stabilization and storage of formulations incorporating beneficial bacteria.

A necessary attribute of any pharmaceutical product is long-term stability (shelf life), typically at least 24 months under predefined storage conditions. To achieve this attribute, an efficient, scalable and reliable manufacturing platform needs to be developed for Bacterial-based Drug Substance (DS) and Drug Product (DP) formulations. During manufacturing and subsequent storage, the critical parameter for product stability is long-term viability. Manufacture, storage and eventual therapeutic use of commensal or engineered microbiome strains impose significant stress on the bacterial cell. In industrial settings, bacteria are preserved and distributed in liquid, spray-dried, frozen or lyophilized (freeze-dried) forms. These preparations are suitable for use as starter cultures in the food industry. However, emphasis is increasingly being placed on long-term preservation methods that promote high cell viability and metabolic activity, as these parameters are considered a prerequisite for (bio)pharmaceutical applications.

In order to maximize survival, it is possible to add one or more selected cryoprotectants to the biomass with subsequent lyophilization, especially considering the fact that viable and metabolically active bacteria have been shown to induce the desired therapeutic effect in situ.

Freeze-drying is widely regarded as a suitable dehydration processes for bacteria, aiming to achieve a solid and stable final formulation. It is one of the most common methods to store microbial cell cultures, even though survival rates after freeze-drying and during storage may vary between strains.

Without intending to be bound by any theory, survival after freeze-drying reflects the ability of the cells to resist the effects of rapid freezing and drying, such as membrane lipid oxidation and cell damage at several target sites. It is well known that the freeze-drying of unprotected bacteria kills most of them, and those that survive, often die rapidly upon storage. Several attempts have been made to increase the number of surviving bacteria upon lyophilization and storage, with limited success. Lyophilization is the most frequently, if not exclusively used method to achieve long-term bacterial shelf life. The choice of an appropriate drying medium/cryoprotectant mixture is critical to increase the survival rate of therapeutic strains of bacteria during lyophilization and subsequent storage. Several studies attempting to increase the survival rate of Lactic Acid Bacteria (LAB) during freeze-drying and/or subsequent storage have been reported, however, few of these publications demonstrate sufficient long-term stability of the freeze-dried bacteria, as required for pharmaceutical applications, and no studies have been identified which demonstrate long-term lyophilization stability for Staphylococcus species or other commensal species known to live on and protect human skin, help skin barrier function, and can modulate skin innate immunity; by secretion of targeted and immune privileged small peptides and fatty acid entities.

Most LAB cultures of commercial interest have historically selected skim milk powder as the drying medium because it stabilizes the cell membrane constituents, facilitates rehydration and forms a protective coating over the cells. Supplementing milk with additional cryoprotectants agents may enhance its intrinsic protective effect. However, cryoprotection of non-Lactobacillus bacteria is not well characterized and methods and compositions for the preservation of Staphylococcus bacteria have not been robustly developed.

Other cryoprotectantants investigated for bacterial cryopreservation in LAB have included adonitol in 10% skim milk; skim milk (11%) compared to trehalose (5%); and sorbitol or (mono)sodium glutamate (MSG), each added separately to LAB suspended in skim milk. Stability was increased compared to skim milk alone, however the reported survival rates in the presence of sorbitol or MSG were still very low (<0.1%). Furthermore, the sugars sucrose and polyols such as mannitol, as well as the amino acid β-alanine and stabilization potentiators like L-ascorbic acid and maltodextrin, as well as non-animal growth media (such as Soytone® (Corning)) are capable of enhancing cell viability above that recorded in skim milk alone.

Skim milk is a recurrent component of freeze-drying media for LAB, and thus appears to be essential for bacterial viability and an important constituent of the stabilized dry bacterial compositions. However, the use of milk and animal derivatives (e.g. bovine serum albumin, trypsin-based peptones) in novel pharmaceutical compositions is strongly discouraged, especially in view of the Transmissible Spongiform Encephalopathy (TSE) risk associated with their use. Few studies have addressed the replacement of the milk components with sufficient survival and stability under long-term shelf storage. In fact, most of these studies lack precise data on initial viability, stability and bacterial density. Finally, none of them report on freeze-drying of Staphylococcus bacteria or other skin commensal microbiota and/or maintenance of their properties to benefit the skin. Disclosed herein are compositions and methods for the production of stable lyophilized compositions incorporating Staphylococcus bacteria and/or other bacteria that are associated with healthy skin microbiota.

Once lyophilization has been achieved, it is necessary to prepare compositions that can be administered to a subject in order to achieve a therapeutic and/or a cosmetic effect. It is further necessary to prepare appropriate placebo substances such that the effects of said therapeutic and/or cosmetic compositions can be studied. Due to the freeze-dried nature of live biotherapeutic drug substances stabilized in hygroscopic excipients, product formulations must be developed with appropriate anhydrous excipients to maintain product shelf-live and therapeutic efficacy without adding water or water vapor which will activate cell metabolism and initiate product breakdown during storage. Stabilized lyophilized lactic acid bacteria in anhydrous food grade vegetable oils are currently marketed for non-prescription use for infants with colic, and have been used for oral probiotic supplements for livestock. These products can maintain stable CFU recovery for 24 months at room temperature storage. With this background, Sh-A9 Drug Product was developed using topical oils and excipients already approved in other topical pharmaceutical products to identify a minimal excipient combination which will not destabilize the drug substance powder.

Without being bound to any particular theory, it is recognized that factors which can influence excipient stability over time and affect product stability (include, for example, oxidization of oil lipids, room humidity during formulation and container closure). Therefore, it is the object to the present disclosure to provide compositions which show stability as defined by the ability to recover bacterial growth (CFU after storage) as well as by reductions in the rate of decomposition (by oxidation, separation, etc.) of the excipients and/or excipient mixtures. In some embodiments, compositions are provided which utilize anhydrous excipients to maintain freeze dried bacterial stability in product formulations. In some embodiments, the compositions disclosed herein may demonstrate room temp storage stability to a 1 day expiration date, a 1 week expiration date, a 1 month expiration date, a 2 month expiration date, a 4 month expiration date, a 6 month expiration date, an 8 month expiration date, a 12 month expiration date, an 18 month expiration date, a 24 month expiration date, or a 36 month expiration date, or a date within a range defined by any of these values.

Exemplary topical oils and topical excipients include, for example, those found in the FDA Inert Ingredient Guide database, accessible at www.accessdata.fda.gov/scripts/cder/iig/index.cfm, last accessed Nov. 13, 2018, which is hereby incorporated by reference in its entirety and particularly with respect to its disclosure of the identity and use of ingredients for the preparation of topical compositions. In some embodiments, compositions are provided which deliver >1×10⁷ CFU/gram at date of expiration, where the death at expiration is determined with regard to the requirements of the national health and safety laws governing said compositions at the time of their making or marketing. 1×10⁷ CFU/gram may, for example, yield 1.2×10⁵ CFU/cm² for a 2 gram application on 100 cm² of skin surface area.

Disclosed herein are compositions and methods for the treatment, amelioration, or prevention of one or more skin disorders or diseases associated with dysbiosis, especially dysbiosis of the skin, or symptoms thereof, or syndromes incorporating said disorders, diseases, or symptoms. Exemplary disorders or diseases to be treated according to the compositions and methods as disclosed herein include, but are not limited to, atopic dermatitis, eczema, pyotraumatic dermatitis, pyoderma, superficial pyoderma, folliculitis, rosacea, Netherton syndrome, acne, wounds (including abrasions, radiation damage, and burns), psoriasis, mastitis, icthyosis, lichen formation, and sebhorreic dermatitis, or any combination thereof. Exemplary symptoms of skin disorders or diseases associated with dysbiosis of the skin include but are not limited to thickened skin; discolored (especially reddish) skin; itching; open sores, blisters, cracks, or lesions that drain fluid, ooze, and/or crust; swelling; red rash or bumps; raw skin from scratching; red, leathery, cracked or scaly patches on the skin; dry, red patches, which may resemble a burn; burning, stinging or itching, which may be mild, moderate, or severe; small red, pus-filled bumps; yellowish scales or crust on the scalp, ears, face or other parts of the body; dandruff; icthyosis, dermal fibrosis, or other symptoms of localized, persistent inflammation or of dermatitis or dermatitis syndromes as are known in the art or any combination thereof. Representative dermatitis syndromes and/or symptoms thereof for which administration of the compositions described herein, preferably according to the methods described herein may comprise one or more of hyper IgE syndrome, pneumatocele, hyperextensibility, osteopenia, aneurysma, hypertension, recurrent infections, Wiskott-Aldrich syndrome, thrombocytopenia, recurrent infections, ectodermal dysplasias, Netherton syndrome, erythroderma, peeling skin disease, hyperhidrosis, clubbing, photophobia, severe dermatitis, multiple allergies, and metabolic wasting (SAM) syndrome, recurrent infections, multiple allergies, ichthyosis follicularis, alopecia, and photophobia (IFAP) syndrome, ichthyosis follicularis, alopecia, and photophobia; minimal change nephrotic syndrome (MCNS); SAM, severe dermatitis, multiple allergies, atopic dermatitis with ichthyosis follicularis, ichthyosis follicularis, atrichia, and/or photophobia, and/or any combination thereof. In some embodiments, the compositions and methods of the present disclosure provide a treatment for atopic dermatitis.

In some embodiments, the compositions and methods of the present disclosure contemplate treatments for diseases or disorders related to dysbiosis of the skin. In some embodiments, the present disclosure contemplates treatment, amelioration, or cure of the underlying dysbiosis. In some embodiments, said dysbiosis comprises a reduction or decrease in the relative amount or the absolute amount of a beneficial, protective, or health-associated bacterium in, on, or associated with the skin or external mucosae or the normal flora associated therewith. In some embodiments, said health-associated bacterium may comprise one or more coagulase negative staphylococci. Coagulase-negative staphylococci which may be present on the skin of a subject may include, for example, Staphylococcus epidermidis, Staphylococcus haemolyticus, Staphylococcus hominis, Staphylococcus capitis, Staphylococcus capitis ureolyticus, Staphylococcus caprae, Staphylococcus articularis, Staphylococcus saccharolyticus, Staphylococcus warneri, Staphylococcus pasteri, Staphylococcus saprophyticus, Staphylococcus cohnii, Staphylococcus cohnii urealyticum, Staphylococcus xylosus, Staphylococcus arlettae, Staphylococcus equorum, Staphylococcus gallinarum, Staphylococcus kloosii, Staphylococcus lentus, Staphylococcus simulans, Staphylococcus carnosus, Staphylococcus schleiferi, Staphylococcus sciuri, Staphylococcus lentus, Staphylococcus vitulinus, Staphylococcus chromogenes, Staphylococcus caseolyticus, Staphylococcus felis, Staphylococcus hyicus, Staphylococcus lugdunensis, Staphylococcus muscae, Staphylococcus piscifermentans, or others as are known in the art, or any combination thereof. In some embodiments, a health-associated bacterium may comprise one or more of Staphylococcus epidermis, Staphylococcus hominis, Staphylococcus lugdunensis, Staphylococcus warneri, and/or Staphylococcus capitis or any combination thereof. In some embodiments, a health-associated bacterium may comprise one or more of Staphylococcus epidermidis, Staphylococcus capitis, Staphylococcus caprae, Staphylococcus saccharolyticus, Staphylococcus warneri, Staphylococcus pasteuri, Staphylococcus haemolyticus, Staphylococcus devriesei, Staphylococcus hominis, Staphylococcus jettensis, Staphylococcus petrasii, and/or Staphylococcus lugdunensis. In some embodiments, said dysbiosis may comprise colonization, infection, overgrowth, or an increase in the presence, relative amount, or absolute amount of a deleterious, infectious, pathogenic, or disease-associated bacterium. In some embodiments, said deleterious, infectious, pathogenic, or disease-associated bacterium may comprise one or more bacteria known in the art to cause or to be associated with infections, diseases, disorders, or pathological conditions, especially inflammations and irritations, of the skin and/or external mucosae. In some embodiments, said deleterious, infectious, pathogenic, or disease-associated bacterium may comprise one or more of Staphylococcus aureus, Staphylococcus schleiferi, Staphylococcus intermedius, Staphylococcus pseudintermedius, Staphylococcus felis, or other bacterial infections such as Mallassezia, especially Mallassezia sympodialis, Mallassezia globosa, Micrococcus spp., Acinetobacter spp., alpha-hemolytic streptococci, and/or other pathogens of the skin or external mucosa.

The compositions and methods disclosed herein contemplate administration of said compositions for the treatment of diseases or disorders related to dysbiosis of the skin in a human subject or in a nonhuman subject, such as a dog, cat, horse, cow, nonhuman primate, etc. The compositions and methods disclosed herein contemplate administration of said compositions for the treatment of diseases or disorders related to dysbiosis of the skin wherein said diseases or disorders related to dysbiosis of the skin may comprise one or more of atopic dermatitis, eczema, pyotraumatic dermatitis, pyoderma, superficial pyoderma, folliculitis, rosacea, Netherton syndrome, acne, wounds (including abrasions, radiation damage, and burns), psoriasis, mastitis, icthyosis, lichen formation, and sebhorreic dermatitis, or any combination thereof, or other such disorders as are known in the art or may be reasonably determined by one of skill in the art to be associated with dysbiosis in human or nonhuman subjects. In some embodiments, said diseases or disorders related to dysbiosis of the skin may comprise one or more of Netherton syndrome, atopic dermatitis, contact dermatitis, eczema, psoriasis, acne, epidermal hyperkeratosis, acanthosis, epidermal inflammation, dermal inflammation and/or pruritus. The compositions and methods disclosed herein contemplate administration of said compositions for the treatment of diseases or disorders related to dysbiosis of the skin wherein said diseases or disorders related to dysbiosis of the skin may be associated with the presence, colonization by, overgrowth of, or activity of one or more of comprise one or more of Staphylococcus aureus, Staphylococcus schleiferi, Staphylococcus intermedius, Staphylococcus pseudintermedius, Staphylococcus felis, or other bacterial infections such as Mallassezia, especially Mallassezia sympodialis, Mallassezia globosa, Micrococcus spp., Acinetobacter spp., alpha-hemolytic streptococci, and/or other pathogens of the skin or external mucosa or other such bacteria as are known in the art to be associated with skin disorders involving dysbiosis in human or nonhuman subjects. The compositions and methods disclosed herein contemplate administration of said compositions for the treatment of diseases or disorders related to dysbiosis of the skin wherein said health-associated bacterium may comprise one or more of Staphylococcus epidermis, Staphylococcus hominis, Staphylococcus lugdunensis, Staphylococcus warneri, and/or Staphylococcus capitus or any combination thereof. The compositions and methods disclosed herein contemplate administration of said compositions to a subject for the treatment of diseases or disorders related to dysbiosis of the skin wherein said subject is a nonhuman animal. The compositions and methods disclosed herein contemplate administration of said compositions to a subject for the treatment of diseases or disorders related to dysbiosis of the skin wherein said subject is a dog, cat, horse, cow, camel, goat, or llama. The compositions and methods disclosed herein contemplate administration of said compositions to a subject for the treatment of diseases or disorders related to dysbiosis of the skin wherein said diseases or disorders related to dysbiosis of the skin may comprise atopic dermatitis and wherein said subject is a human subject. The compositions and methods disclosed herein contemplate administration of said compositions to a subject for the treatment of diseases or disorders related to dysbiosis of the skin wherein said diseases or disorders related to dysbiosis of the skin may comprise pyotraumatic dermatitis, superficial pyoderma, and/or folliculitis, and wherein said subject is a nonhuman subject. The compositions and methods disclosed herein contemplate administration of said compositions to a subject for the treatment of diseases or disorders related to dysbiosis of the skin wherein said diseases or disorders related to dysbiosis of the skin may comprise pyotraumatic dermatitis, superficial pyoderma, and/or folliculitis, and wherein said subject is a dog or a cat. The compositions and methods disclosed herein contemplate administration of said compositions to a subject for the treatment of diseases or disorders related to dysbiosis of the skin wherein said diseases or disorders related to dysbiosis of the skin may comprise pyotraumatic dermatitis and/or mastitis, and wherein said subject is a cow, horse, goat, or camel.

The compositions disclosed herein may comprise an active drug substance or an active cosmetic substance wherein said active drug substance or active cosmetic substance has the effect of ameliorating or curing dysbiosis in a tissue or at a particular location in a subject. The effect of ameliorating or curing dysbiosis in a tissue or at a particular location in a subject may comprise any of the steps of reducing the presence, activity, or virulence of a pathogen, an overgrown bacterium, or a disease-associated bacterium; increasing the presence activity, or beneficial effects of a commensal, beneficial, or health-associated bacterium; enhancing the stability of a bacterial community; altering the production, secretion, or availability of one or more metabolites; altering the utilization of a food source, electron acceptor, electron donor, ion or counterion; or any other action which may alter the status of the flora or microbiota of a subject, especially where such alteration has an effect on the health, appearance, or comfort of the subject.

The compositions disclosed herein may comprise one or more forms intended for administration to a subject, especially by topical administration. In some embodiments, a topical formulation disclosed herein can be in a form selected from the group consisting of liquid, including solution and suspension, solid, foamy material, emulsion, paste, gel, cream, and a combination thereof, such as a liquid containing a certain amount of solid contents. In some embodiments, the flavoring concentrate formulation may be a fluid, gel, or thickened form which may be aqueous-based or nonaqueous-based. In some embodiments, the compositions disclosed herein may comprise an oil. In some embodiments, the compositions disclosed herein may comprise a lotion. In some embodiments, the compositions disclosed herein may comprise a mist, spray, rinse, liquid, solid stick, roll-on, powder, or other composition as is known in the art or reasonably could be utilized for the administration of active drug compounds to sites of inflammation and/or infection on the skin.

The compositions of the present disclosure may comprise, in some embodiments, an extract, lysate, growth medium, conditioned growth medium, or active isolate thereof, from coagulase negative Staphylococcus (CoNS) bacterial cells. One of ordinary skill could readily isolate an extract, lysate, growth medium, conditioned growth medium, or active isolate thereof, from coagulase negative Staphylococcus (CoNS) bacterial cells using any suitable method known in the art. In one non-limiting example, the bacterial cell can be disrupted by mechanical means. The resulting lysate or suspension may then be processed to remove undesired solids. The isolate may then be placed in a shallow vessel and quickly exposed to low temperature, i.e., flash frozen, for example at −20° C. or lower, preferably under a vacuum for removal of water content (lyophilization). In some embodiments, this process is carried out with a bacterial cell population. In some embodiments, this process is carried out with a bacterial cell lysate. In some embodiments, this process is carried out with a bacterial growth medium.

In other aspects, aqueous, alcoholic, or oil based extraction techniques, or combinations thereof, can be used on the whole bacteria or extracts or derivatives thereof of (e.g., conditioned broth, sub-fractions from lysed or resuspended cells, or conditioned broth, etc.) to produce an extract. In such a process, the desired bacterial extract, broth, or the whole bacterial cell may optionally be crushed or mechanically or chemically disturbed, and then subjected to a desired solvent (e.g., water, alcohol, water/alcohol, or oil based solvents) to obtain an extract. The extract can then be stored in liquid form, lyophilized, or subject to further processing techniques (e.g., heating, cooling, etc.). Extraction processes are well-known to those having ordinary skill and may include or incorporate one or more of maceration, infusion, percolation, digestion, decoction, hot continuous extraction, aqueous-alcoholic extract, counter current extraction, microwave assisted extraction, ultrasound extraction, supercritical fluid extraction, phytonic extract (e.g., with hydro-fluoro-carbon solvents, TFF, bulk column chromatography, etc.

It is contemplated that the compositions of the present invention can include an extract of coagulase negative Staphylococcus (CoNS) which may include, but is not limited to, one or more of Staphylococcus hominis, Staphylococcus epidermidis, Staphylococcus capitis, Staphylococcus warneri Staphylococcus lugdunensis, or Staphylococcus haemolyticus, or any combination thereof. The compositions may also include additional ingredients described elsewhere herein. The concentrations of the bacterial extracts and/or additional ingredients can vary. In some embodiments, the compositions can include in their final form, for example, at least 0.0001%, 0.0002%, 0.0003%, 0.0004%, 0.0005%, 0.0006%, 0.0007%, 0.0008%, 0.0009%, 0.0010%, 0.0011%, 0.0012%, 0.0013%, 0.0014%, 0.0015%, 0.0016%, 0.0017%, 0.0018%, 0.0019%, 0.0020%, 0.0021%, 0.0022%, 0.0023%, 0.0024%, 0.0025%, 0.0026%, 0.0027%, 0.0028%, 0.0029%, 0.0030%, 0.0031%, 0.0032%, 0.0033%, 0.0034%, 0.0035%, 0.0036%, 0.0037%, 0.0038%, 0.0039%, 0.0040%, 0.0041%, 0.0042%, 0.0043%, 0.0044%, 0.0045%, 0.0046%, 0.0047%, 0.0048%, 0.0049%, 0.0050%, 0.0051%, 0.0052%, 0.0053%, 0.0054%, 0.0055%, 0.0056%, 0.0057%, 0.0058%, 0.0059%, 0.0060%, 0.0061%, 0.0062%, 0.0063%, 0.0064%, 0.0065%, 0.0066%, 0.0067%, 0.0068%, 0.0069%, 0.0070%, 0.0071%, 0.0072%, 0.0073%, 0.0074%, 0.0075%, 0.0076%, 0.0077%, 0.0078%, 0.0079%, 0.0080%, 0.0081%, 0.0082%, 0.0083%, 0.0084%, 0.0085%, 0.0086%, 0.0087%, 0.0088%, 0.0089%, 0.0090%, 0.0091%, 0.0092%, 0.0093%, 0.0094%, 0.0095%, 0.0096%, 0.0097%, 0.0098%, 0.0099%, 0.0100%, 0.0200%, 0.0250%, 0.0275%, 0.0300%, 0.0325%, 0.0350%, 0.0375%, 0.0400%, 0.0425%, 0.0450%, 0.0475%, 0.0500%, 0.0525%, 0.0550%, 0.0575%, 0.0600%, 0.0625%, 0.0650%, 0.0675%, 0.0700%, 0.0725%, 0.0750%, 0.0775%, 0.0800%, 0.0825%, 0.0850%, 0.0875%, 0.0900%, 0.0925%, 0.0950%, 0.0975%, 0.1000%, 0.1250%, 0.1500%, 0.1750%, 0.2000%, 0.2250%, 0.2500%, 0.2750%, 0.3000%, 0.3250%, 0.3500%, 0.3750%, 0.4000%, 0.4250%, 0.4500%, 0.4750%, 0.5000%, 0.5250%, 0.550%, 0.5750%, 0.6000%, 0.6250%, 0.6500%, 0.6750%, 0.7000%, 0.7250%, 0.7500%, 0.7750%, 0.8000%, 0.8250%, 0.8500%, 0.8750%, 0.9000%, 0.9250%, 0.9500%, 0.9750%, 1.0%, 1.1%, 1.2%, 1.3%, 1.4%, 1.5%, 1.6%, 1.7%, 1.8%, 1.9%, 2.0%, 2.1%, 2.2%, 2.3%, 2.4%, 2.5%, 2.6%, 2.7%, 2.8%, 2.9%, 3.0%, 3.1%, 3.2%, 3.3%, 3.4%, 3.5%, 3.6%, 3.7%, 3.8%, 3.9%, 4.0%, 4.1%, 4.2%, 4.3%, 4.4%, 4.5%, 4.6%, 4.7%, 4.8%, 4.9%, 5.0%, 5.1%, 5.2%, 5.3%, 5.4%, 5.5%, 5.6%, 5.7%, 5.8%, 5.9%, 6.0%, 6.1%, 6.2%, 6.3%, 6.4%, 6.5%, 6.6%, 6.7%, 6.8%, 6.9%, 7.0%, 7.1%, 7.2%, 7.3%, 7.4%, 7.5%, 7.6%, 7.7%, 7.8%, 7.9%, 8.0%, 8.1%, 8.2%, 8.3%, 8.4%, 8.5%, 8.6%, 8.7%, 8.8%, 8.9%, 9.0%, 9.1%, 9.2%, 9.3%, 9.4%, 9.5%, 9.6%, 9.7%, 9.8%, 9.9%, 10%, 11%, 12%, 13%, 14%, 15%, 16%, 17%, 18%, 19%, 20%, 21%, 22%, 23%, 24%, 25%, 26%, 27%, 28%, 29%, 30%, 35%, 40%, 45%, 50%, 60%, 65%, 70%, 75%, 80%, 85%, 90%, 95%, or 99% or more than 99%, or less than 0.0001%, or any range or integer derivable therein, of at least one of the CONS extracts identified herein. In non-limiting aspects, the percentage of such ingredients can be calculated by weight or volume of the total weight of the compositions. The concentrations can vary depending on the desired effect of the compositions or on the product into which the compositions are incorporated.

The compositions disclosed herein can be formulated into any appropriate vehicle. Non-limiting examples of suitable vehicles include oils, emulsions (e.g., oil-in-water, water-in-oil, silicone-in-water, water-in-silicone, water-in-oil-in-water, oil-in-water, oil-in-water-in-oil, oil-in-water-in-silicone, etc.), suspensions, creams, lotions, anhydrous lotions, solutions (both aqueous, oil, and hydro-alcoholic), anhydrous bases (such as lipsticks and powders), gels, ointments, pastes, milks, liquids, aerosols, solid forms, eye jellies, or skin-serums. Variations and other appropriate vehicles will be apparent to the skilled artisan and are appropriate for use in the present invention. In certain aspects, the concentrations and combinations of the ingredients can be selected in such a way that the combinations are chemically compatible and do not cause degradation, oxidation, etc. of the active drug product.

It is also contemplated that the CoNS extracts and additional ingredients identified throughout this specification may be encapsulated for delivery to a target area such as skin. Non-limiting examples of encapsulation techniques include the use of liposomes, vesicles, and/or nanoparticles (e.g., biodegradable and non-biodegradable colloidal particles comprising polymeric materials in which the ingredient is trapped, encapsulated, and/or absorbed—examples include nanospheres and nanocapsules) that can be used as delivery vehicles to deliver such ingredients to skin. Representative nanoparticles and/or nanocapsules are described in U.S. Pat. Nos. 6,387,398; 6,203,802, and 5,411,744, which are hereby incorporated by reference with respect to their disclosure of the making, use, administration, and storage of nanoparticles and nanocapsules and related compositions for topical administration.

For purposes of the present disclosure, the following definitions are provided.

As used herein, when referring to the term “dysbiosis” means an imbalance or maladaptation of the flora or microbiota within one or more tissues, compartments, subcompartments, or locations of the body, and particularly within the various domains and subdomains of the skin. Such dysbiosis is characterized by a change in the composition of the local microbiome, in terms of the species/strains which are present and/or the relative abundance or proportion of the species/strains which are present, in which the change has a definable effect on the host organism. The effect on the host organism can result from microbiome-mediated changes in electrolyte balance, biofilm formation, epithelial, mesothelial, or endothelial barrier integrity, or the release from the microbiome of metabolites which directly (e.g., as toxins or effectors) or indirectly (e.g., as pre-cursors to toxins or effectors) affect the health, appearance, or comfort of the host.

As used herein, an “active drug substance” refers to any substance, compound, or composition that is capable of or intended to have an effect in the treatment, amelioration, or cure of dysbiosis in a subject; or of any disease, disorder, or condition related to dysbiosis in a subject, or that is otherwise intended to affect the health, comfort, or appearance of a subject. In some embodiments, an active drug substance as disclosed herein may comprise one or more of a bacterium, a killed bacterium, a bacterial culture, a bacterial extract, a growth medium, a lyophilized bacterium, a lyophilized killed bacterium, a lyophilized bacterial culture, a lyophilized bacterial extract, a lyophilized growth medium, or any equivalent or derivative thereof, or any combination of the foregoing. In some embodiments, an active drug substance may be provided for use in treating, ameliorating, or curing dysbiosis in a subject; or any disease, disorder, or condition related to dysbiosis in a subject, or otherwise in affecting the health, comfort, or appearance of a subject. In some embodiments, an active drug substance may be provided for the manufacture of a medicament for use in treating, ameliorating, or curing dysbiosis in a subject; or any disease, disorder, or condition related to dysbiosis in a subject, or otherwise in affecting the health, comfort, or appearance of a subject. Where a substance, compound, or composition is provided primarily for the purpose of altering or improving the appearance of a subject, without regard to any effects said substance, compound, or composition may have on the treatment, amelioration, or cure of dysbiosis in said subject, the term “active cosmetic substance” may optionally be used. The present disclosure contemplates the use of active drug substances for cosmetic purposes and vice versa.

As used herein, the term “biocompatible” refers to a composition does not have clinically significant toxic or injurious effects, locally or systemically. The term “biocompatible” does not exclude the possibility that a composition may have one or more effects on the health, appearance, or comfort of a subject which do not rise to the level of clinical significance or are acceptable in comparison to the severity of other symptoms or conditions.

As used herein, the term “nonabsorbable” refers to a composition, component thereof, or a compound that is substantially incapable of being absorbed across the tissue layer to which it is administered, such as the skin, oral mucosa, nasal/nasopharyngeal mucosa, optic mucosae, orbital mucosae and ocular surfaces, vaginal mucosae, or gastrointestinal epithelia, or other such mucosal, epithelial, or boundary layers, such that less than 25%, and preferably less than 20%, 15%, 10%, 5% or 1% of the composition, either by weight or by volume is systemically absorbed.

As used herein, “stable” refers to a composition that retains any amount of one or more elements essential to its function, at a time after it was originally made, manufactured, formulated, assembled, or packaged. A stable formulation as disclosed herein may, for example, retain up to 10%, up to 20%, up to 30%, up to 40%, up to 50%, up to 60%, up to 70%, up to 80%, up to 90%, or up to 100% of its activity in treating, ameliorating, or preventing one or more skin disorders or diseases associated with dysbiosis. Where said formulation comprises one or more live, lyophilized, freeze-dried, encapsulated, or otherwise reconstitutable bacteria as disclosed herein, a stable formulation may be one which retains up to 10%, up to 20%, up to 30%, up to 40%, up to 50%, up to 60%, up to 70%, up to 80%, up to 90%, or up to 100% of its colony forming activity relative to the composition as it was originally made, manufactured, formulated, assembled, or packaged. A stable formulation as disclosed herein may further retard or prevent the growth of or colonization by undesirable microorganisms, such as those not intended to be present in the formulation or those which may contaminate or disrupt the formulation. Stability may be achieved by any means known in the art and especially by those methods and compounds as disclosed herein. The use of sterile or aseptic technique in the making, manufacture, and/or packaging of the compositions may contribute to the stability of said compositions, as may the elements included within said composition and/or the elements, systems, and mechanisms of the packaging, storage or dispensation of said compositions.

In some embodiments, the compositions disclosed herein may be stable for 1, 2, 3, 4, 5, 6, 7, 8, 9, 10, 11, 12, 13, 14, 15, 16, 17, 18, 19, 20, 21, 22, 23, or 24 months, or more, when stored at or below room temperature. In some embodiments, the compositions disclosed herein may be stable for 1, 2, 3, 4, 5, 6, 7, 8, 9, 10, 11, 12, 13, 14, 15, 16, 17, 18, 19, 20, 21, 22, 23, or 24 months, or more, when stored at or below 40° C. In some embodiments, the compositions disclosed herein may be stable for 1, 2, 3, 4, 5, 6, 7, 8, 9, 10, 11, 12, 13, 14, 15, 16, 17, 18, 19, 20, 21, 22, 23, or 24 months, or more, when stored at or below 37° C. In some embodiments, the compositions disclosed herein may be stable for 1, 2, 3, 4, 5, 6, 7, 8, 9, 10, 11, 12, 13, 14, 15, 16, 17, 18, 19, 20, 21, 22, 23, or 24 months, or more, when stored at or below 25° C. In some embodiments, the compositions disclosed herein may be stable for 1, 2, 3, 4, 5, 6, 7, 8, 9, 10, 11, 12, 13, 14, 15, 16, 17, 18, 19, 20, 21, 22, 23, or 24 months, or more, when stored at or below 20° C. In some embodiments, the compositions disclosed herein may be stable for 1, 2, 3, 4, 5, 6, 7, 8, 9, 10, 11, 12, 13, 14, 15, 16, 17, 18, 19, 20, 21, 22, 23, or 24 months, or more, when stored at or below 15° C. In some embodiments, the compositions disclosed herein may be stable for 1, 2, 3, 4, 5, 6, 7, 8, 9, 10, 11, 12, 13, 14, 15, 16, 17, 18, 19, 20, 21, 22, 23, or 24 months, or more, when stored at or below 10° C. In some embodiments, the compositions disclosed herein may be stable for 1, 2, 3, 4, 5, 6, 7, 8, 9, 10, 11, 12, 13, 14, 15, 16, 17, 18, 19, 20, 21, 22, 23, or 24 months, or more, when stored at or below 4° C. In some embodiments, the compositions disclosed herein may be stable for 1, 2, 3, 4, 5, 6, 7, 8, 9, 10, 11, 12, 13, 14, 15, 16, 17, 18, 19, 20, 21, 22, 23, or 24 months, or more, when stored at or below 0° C. In some embodiments, the compositions disclosed herein may be stable for 1, 2, 3, 4, 5, 6, 7, 8, 9, 10, 11, 12, 13, 14, 15, 16, 17, 18, 19, 20, 21, 22, 23, or 24 months, or more, when stored at any temperature between, or at temperatures that fluctuate anywhere between, 0° C. and 40° C. In some embodiments, the compositions disclosed herein may be stable for 1, 2, 3, 4, 5, 6, 7, 8, 9, 10, 11, 12, 13, 14, 15, 16, 17, 18, 19, 20, 21, 22, 23, or 24 months, or more, when stored at any temperature between, or at temperatures that fluctuate anywhere between, 0° C. and 37° C. In some embodiments, the compositions disclosed herein may be stable for 1, 2, 3, 4, 5, 6, 7, 8, 9, 10, 11, 12, 13, 14, 15, 16, 17, 18, 19, 20, 21, 22, 23, or 24 months, or more, when stored at any temperature between, or at temperatures that fluctuate anywhere between, 0° C. and 30° C. In some embodiments, the compositions disclosed herein may be stable for 1, 2, 3, 4, 5, 6, 7, 8, 9, 10, 11, 12, 13, 14, 15, 16, 17, 18, 19, 20, 21, 22, 23, or 24 months, or more, when stored at any temperature between, or at temperatures that fluctuate anywhere between, 0° C. and 25° C. In some embodiments, the compositions disclosed herein may be stable for 1, 2, 3, 4, 5, 6, 7, 8, 9, 10, 11, 12, 13, 14, 15, 16, 17, 18, 19, 20, 21, 22, 23, or 24 months, or more, when stored at any temperature between, or at temperatures that fluctuate anywhere between, 0° C. and 20° C. In some embodiments, the compositions disclosed herein may be stable for 1, 2, 3, 4, 5, 6, 7, 8, 9, 10, 11, 12, 13, 14, 15, 16, 17, 18, 19, 20, 21, 22, 23, or 24 months, or more, when stored at any temperature between, or at temperatures that fluctuate anywhere between, 0° C. and 15° C. In some embodiments, the compositions disclosed herein may be stable for 1, 2, 3, 4, 5, 6, 7, 8, 9, 10, 11, 12, 13, 14, 15, 16, 17, 18, 19, 20, 21, 22, 23, or 24 months, or more, when stored at any temperature between, or at temperatures that fluctuate anywhere between, 0° C. and 10° C. In some embodiments, the compositions disclosed herein may be stable for 1, 2, 3, 4, 5, 6, 7, 8, 9, 10, 11, 12, 13, 14, 15, 16, 17, 18, 19, 20, 21, 22, 23, or 24 months, or more, when stored at any temperature between, or at temperatures that fluctuate anywhere between, 0° C. and 4° C.

As used herein, the term “associated” means that the presence or level of a bacterium, bacterial consortium, combination of bacteria, metabolite, reaction product of the metabolite, extract, or other composition or phenomenon has been statistically significantly correlated with the presence or degree of the symptom or disorder, and/or that the bacterium, bacterial consortium, combination of bacteria, metabolite, reaction product of a metabolite, extract, or other composition or phenomenon, has been causally or mechanistically related to the development, maintenance or degree of the symptom or disorder. The determination of statistical significance can be made according to appropriate statistical models or tests as identified by one of ordinary skill in the art, with the threshold for significance determined likewise as appropriate to the particular data by one of ordinary skill

The term “subject” as used herein, refers to a human, other mammal, or a non-human animal including but not limited to a dog, cat, horse, donkey, chicken, duck, goose, turkey, guinea fowl, emu, ostrich, parrot, canary, mynah bird, pheasant, quail, partridge, peafowl, mule, cow, domestic buffalo, camel, llama, alpaca, bison, yak, goat, sheep, pig, elk, deer, domestic antelope, or a non-human primate selected or identified for removal of one or more microbial metabolites (and host-generated modifications of these metabolites) or selected or identified for treatment, inhibition, or amelioration of a disease, disorder, or condition, or any symptom thereof, including cosmetic conditions, associated with an alteration in the microbiome of the subject, especially with respect to the microbiome of the skin, intestine, eye, oral mucosa, nasal mucosa, respiratory mucosa, ear, cloaca, or vagina. Particular diseases or disorders may include without limitation any diseases or disorders disclosed herein, such as atopic dermatitis, eczema, pyotraumatic dermatitis, pyoderma, superficial pyoderma, folliculitis, rosacea, Netherton syndrome, acne, wounds (including abrasions, radiation damage, and burns), psoriasis, mastitis, icthyosis, lichen formation, and sebhorreic dermatitis, or any combination thereof.

The term “subject suspected of having” refers to a subject exhibiting one or more symptoms, cosmetic indicators, or clinical indicators of a disease, disorder, or condition. In some embodiments, the disease, disorder, or condition may comprise one or more of atopic dermatitis, eczema, pyotraumatic dermatitis, pyoderma, superficial pyoderma, folliculitis, rosacea, Netherton syndrome, acne, wounds (including abrasions, radiation damage, and burns), psoriasis, mastitis, icthyosis, lichen formation, and sebhorreic dermatitis, or any combination thereof. In some embodiments, said one or more symptoms, cosmetic indicators, or clinical indicators of a disease, disorder, or condition may be established based on the appearance of the subject. In some embodiments, exhibition one or more symptoms, cosmetic indicators, or clinical indicators of a disease, disorder, or condition is determined by one of skill in the art of diagnosis and/or treatment of dermatological diseases, disorders, or conditions. In some embodiments, exhibition of one or more symptoms, cosmetic indicators, or clinical indicators of a disease, disorder, or condition is determined by the subject. In some embodiments, exhibition of one or more symptoms, cosmetic indicators, or clinical indicators of a disease, disorder, or condition is determined by a person other than the subject. In some embodiments, exhibition of one or more symptoms, cosmetic indicators, or clinical indicators of a disease, disorder, or condition is determined by a system, algorithm, computer, or software.

The term “subject in need thereof” refers to a subject selected or identified as one being in need of a composition that inhibits dysbiosis or restores a healthy microbiome, or one in need of treatment, inhibition, or amelioration of a disease or disorder, or any symptom thereof, associated with an alteration in the microbiome of the subject, especially with respect to the microbiome of the skin, intestine, eye, oral mucosa, nasal mucosa, respiratory mucosa, ear, or vagina. Particular diseases or disorders may include without limitation atopic dermatitis, eczema, pyotraumatic dermatitis, pyoderma, superficial pyoderma, folliculitis, rosacea, Netherton syndrome, acne, wounds (including abrasions, radiation damage, and burns), psoriasis, mastitis, icthyosis, lichen formation, and sebhorreic dermatitis, or any combination thereof. In some embodiments, a subject in need thereof may comprise a subject showing alterations in appearance due to one or more diseases, disorders, or conditions as disclosed herein. In some embodiments, a subject in need thereof may comprise a subject that desires of amelioration of a cosmetic effect or cosmetic symptom of one or more diseases, disorders, or conditions as disclosed herein.

The term “administering” refers to providing a composition to a subject, such as by contacting the body, including the skin, of the subject with said composition, and includes, but is not limited to, administering by a medical professional and self-administration. Administration of the compositions disclosed herein, including bacteria, bacterial extracts, lysates, metabolites, and mixtures and compositions thereof, can be via any of the accepted modes of administration for agents that serve similar utilities including, but not limited to, topically, ocularly, or ophthalmically, nasally, orally, intraperitoneally, vaginally, or rectally. Topical administrations are customary in administering the compositions that are the subject of particular embodiments as described herein. However in some embodiments, the compositions are administered rectally, nasally, orally, or ophthalmically. In some embodiments, administration may be by a lotion, cream, ointment, unguent, oil, suspension, emulsion, or solution. In some embodiments, administration may be by an enema or suppository. In some embodiments, administration of the compounds may occur systemically, such as by parenteral injection or infusion.

A “therapeutic effect” relieves or alleviates, to at least some extent, one or more of the symptoms of a disease or disorder, and includes curing the disease or disorder. “Curing” means that the symptoms of active disease are eliminated. However, certain long-term or permanent effects of the disease may exist even after a cure is obtained (such as scarring and/or tissue damage).

The term “Colony Forming Unit” (CFU) has its ordinary meaning in the art.

The term “amelioration” refers to a lessening of severity of at least one indicator of a disease, disorder, condition or symptom thereof. In some embodiments, amelioration includes a delay or slowing in the progression of one or more indicators of a disease, disorder, condition or symptom thereof. The severity of indicators can be determined by subjective or objective measures which are known to those skilled in the art. Indicators of a disease, disorder, condition, or symptom thereof, include cosmetic indicators.

The term “modulation” refers to an alteration of function or activity. In some embodiments, modulation refers to an increase in gene expression. In some embodiments, modulation refers to a decrease in gene expression. In some embodiments, modulation refers to an increase or decrease in total serum levels of a specific protein. In some embodiments, modulation refers to an increase or decrease in free serum levels of a specific protein. In some embodiments, modulation refers to an increase or decrease in total serum levels of a specific non-protein factor, e.g., a metabolite. In some embodiments, modulation refers to an increase or decrease in free serum levels of a specific non-protein factor. In some embodiments, modulation refers to an increase or decrease in total bioavailability of a specific protein. In some embodiments, modulation refers to an increase or decrease in total bioavailability of a specific non-protein factor. In some embodiments, modulation refers to an increase or decrease in the relative or absolute abundance of one or more microbial species or microbial strains. In some embodiments, modulation refers to an increase or decrease in the relative or absolute abundance of one or more bacterial species or strains.

As used herein, the term “adjuvant” denotes an additive that supplements, stabilizes, maintains, or enhances the intended function or effectiveness of the active ingredient, such as the compound of the present invention. In some embodiments, the compositions disclosed herein may further comprise one or more adjuvants, which may comprise, consist essentially of, or consist of topically acceptable materials that may be used to preserve or alter the properties of the topical composition disclosed herein. These materials may include, but are not limited to, materials having anti-acne, anti-aging, anti-wrinkle, antifungal, anti-inflammatory, antimicrobial, antioxidant, antiperspirant, antidandruff, anti-dermatitis, antipruritic, anti-emetic, anti-dry skin, anti-psoriatic, anti-seborrheic, anti-asthmatic, astringents, bronchodilating, biocidal, exfoliant (either chemical or physical), cleansing, coloring, corticosteroid, deodorant, depigmenting, depilating, emollient, epilating, analgesic, hair conditioning, hormonal, humectant, light-interacting, luster-imparting, make-up removing, pH adjusting, powder-like, rheology modifying, shine-imparting, skin bleaching, skin conditioning, skin protecting, tanning, UV screening vitamin, and/or wound-healing properties.

In some embodiments, the compositions to be administered according to the methods described herein may comprise, consist essentially of, or consist of one or more of a bacterial strain or a lyophile, growth medium, extract, metabolite, or derivative thereof. In some embodiments, said bacterial strain may comprise one or more members of the genus Staphylococcus. In some embodiments, said bacterial strain may comprise one or more coagulase negative staphylococci. In some embodiments, said bacterial strain may comprise one or more bacteria of the species Staphylococcus hominis. In some embodiments, said bacterial strain may comprise one or more bacteria corresponding to or belonging to Staphylococcus hominis strain A9.

The beneficial properties of pharmaceutical or cosmetic compositions containing at least one of said strains may be further enhanced by addition of one or more additional strains or preparations thereof, including bacteria, cultures, isolates, culture media or fractions thereof, extracts or fractions thereof, lyophiles, metabolites, and/or secreted factors such as, for example, antimicrobial peptides, signaling peptides, autoinducing peptides, lantibiotics, and/or 6-hydroxyaminopurine. Said strains may comprise one or more of Staphylococcus epidermidis strains M034, M038, All, AMT1, AMTS-05, and/or AMTS-G6 and/or Staphylococcus hominis strains A9, C2, C4, C5, C6 AMT2, AMT3, AMT4-C2, AMT4-G1, and/or AMT4-D12, as described in U.S. Patent Application Publication No. 2018/0289751 which is hereby incorporated by reference in its entirety for all purposes and especially for its disclosure of strains and strain characteristics of S. hominis and S. epidermis strains.

In some embodiments, the compositions and methods of the present disclosure may comprise an active particle, which may comprise, consist essentially of, or consist of a particle, such as a lyophilized particle, derived from a cell, a microbial cell, a cell culture, a microbial culture, a culture extract, or a microbial culture extract. Said particle may be a particle, microparticle, or nanoparticle. Said particle may be the result of milling and sieving, including wet milling, and especially wet milling in oil or lipid based or anhydrous media, spray drying, electrospray, electrospinning, or any other method as is known in the art for the production of particles, especially particles for pharmaceutical or cosmetic use. In some embodiments, a milled particle may have an average particle size of 5-40 nm, 25-100 nm, 50-300 nm, 150-500 nm, 300 nm-1 μm, 0.5 μm-2 μm, 1 μm-5 μm, 2.5-10 μm, 6-20 μm, 15-50 μm, 30-100 μm, 75-150 μm, 100-300 μm, 250-500 μm, 300-750 μm, 600 μm-1 mm, or greater than 1 mm or a size that is within a range defined by any two of the aforementioned sizes. In some embodiments, said milled particles have particle sizes of 300 μm-1 mm, 1-3 mm, 2-5 mm, or greater than 5 mm or a size that is within a range defined by any two of the aforementioned sizes. Said active particles may also comprise a plurality of pores and may have a specific surface area in the range of from 20 m²/g to 5000 m²/g, such as, e.g., 20, 50, 100, 250, 500, 750, 1000, 1500, 2000, 2500, 3000, 3500, 4000, 4500 or 5000 m²/g or a specific surface area within a range defined by any two of the aforementioned surface areas. Specific surface area can be determined using known methods, such as, for example, the method of Bruanauer, Emmett and Teller (J. Am. Chem. Soc. (1938), 60:309-311) and/or mercury porosimetry. See, e.g., ASTM Test Methods D3663, D6556, and D4567, each of which is incorporated by reference in its entirety.

Said particles, nanoparticles, or microparticles may additionally have a specific pore volume (determined on the basis of pores having a diameter of 1.0 nm to 100 nm) that is from 0.1 cm³/g to 1.5 cm³/g, from 0.1 cm³/g to 0.8 cm³/g, from 0.1 cm³/g to 0.7 cm³/g, from 0.1 cm³/g to 0.6 cm³/g, from 0.1 cm³/g to 0.5 cm³/g, from 0.2 cm³/g to 0.8 cm³/g, from 0.2 cm³/g to 0.7 cm³/g, from 0.2 cm³/g to 0.6 cm³/g, from 0.2 cm³/g to 0.5 cm³/g, from 0.3 cm 3 /g to 1 cm³/g, from 0.3 cm³/g to 0.9 cm³/g, from 0.3 cm³/g to 0.8 cm³/g, from 0.3 cm³/g to 0.7 cm³/g, from 0.3 cm³/g to 0.6 cm³/g, or from 0.3 cm³/g to 0.5 cm³/g or within a range defined by any two of the aforementioned values, as measured by a method for determining pore diameters and specific pore volumes, such as that described in Barrett, Joyner and Halenda (1951), J. Am. Chem. Soc. 73:373-380 and ASTM D4222-03 (2008) (the method referred to herein as the “BJH method”), both of which are expressly incorporated herein by reference in their entireties, and by the method of mercury porosimetry {e.g., using a mercury porosimeter, such as, for example, the Micromeritics Autopore V 9605 Mercury Porosimeter (Micromeritics Instrument Corp., Norcross, Ga.) in accordance with the manufacturer's instructions). See e.g., ASTM 3663, ASTM D-4284-12 and D6761-07 (2012), all of which are incorporated herein by reference. Said particles, nanoparticles, or microparticles may further have a mean pore diameter in the range of from 2 nm to 100 nm, as measured by the BJH method and/or mercury porosimetry. More typically, the particles, nanoparticles, or microparticles may have a mean pore diameter in the range of from 2-5 nm, from 3-9 nm, from 6-15 nm, from 10 nm to 90 nm or a size that is within a range defined by any two of the aforementioned sizes, as measured by the BJH method and/or mercury porosimetry. In some embodiments, the mean pore diameter is in the range of from 10 nm to 80 nm, or from 10 nm to 70 nm, or from 10 nm to 60 nm, and often from 10 nm to 50 nm or a size that is within a range defined by any two of the aforementioned sizes, as determined by the BJH method and/or mercury porosimetry. In some embodiments, the mean pore diameter is in the range of from 20 nm to 100 nm or a size that is within a range defined by any two of the aforementioned sizes, as measured by the BJH method and/or mercury porosimetry. In certain of these embodiments, the mean pore diameter is in the range from 20 nm to 90 nm, or from 20 nm to 80 nm, or from 20 nm to 70 nm, or from 20 nm to 60 nm, or from 10 nm to 50 nm or a size that is within a range defined by any two of the aforementioned sizes, as determined by the BJH method and/or mercury porosimetry.

The term “agent” includes any substance, microorganism, combination of microorganisms, mixture, molecule, element, compound, entity, or a combination thereof. It includes, but is not limited to, e.g., bacteria, fungi, viruses or phage, lipopolysaccharide, microbial extract, microbial growth medium, protein, polypeptide, peptide or mimetic, small organic molecule, polysaccharide, polynucleotide, polymer, resin, organic or inorganic microparticle, organic or inorganic nanoparticle, and the like. It can be a natural product, a synthetic compound, or a chemical compound, or a combination of two or more substances.

The compositions described herein can be formulated into pharmaceutical compositions, cosmetic compositions, personal care compositions, and/or supplements for use in treating, inhibiting, or ameliorating a disease or disorder associated with an alteration in the skin microbiome such as atopic dermatitis, eczema, pyotraumatic dermatitis, pyoderma, superficial pyoderma, folliculitis, rosacea, Netherton syndrome, acne, wounds (including abrasions, radiation damage, and burns), psoriasis, mastitis, icthyosis, and sebhorreic dermatitis, or any combination thereof. Standard pharmaceutical, cosmetic, personal care, and/or dietary supplement formulation techniques are used, such as those disclosed in Remington's The Science and Practice of Pharmacy, 21st Ed., Lippincott Williams & Wilkins (2005), incorporated herein by reference in its entirety. Accordingly, some embodiments include pharmaceutical, cosmetic, personal care, and/or dietary supplement compositions comprising, consisting essentially of, or consisting of: (a) a safe and therapeutically effective amount of one or more compounds described herein, or pharmaceutically acceptable salts thereof; and (b) a pharmaceutically acceptable carrier, diluent, excipient or combination thereof.

The term “pharmaceutically acceptable carrier” or “pharmaceutically acceptable excipient” includes any and all solvents, cosolvents, diluents, emulsifiers, binders, adsorbents, permeation enhancers, surfactants, stabilizers, preservatives, cheating agents, thickeners, smoothing agents, abrasives, polymers, humectants, emollients, moisturizers, buffers, dispersion media, coatings, antibacterial and antifungal agents, isotonic and absorption delaying agents and the like, or any other such compound as is known by those of skill in the art to be useful in preparing pharmaceutical formulations. As used herein, an excipient or pharmaceutically acceptable excipient, or a cosmetically acceptable excipient, may comprise any component of a formulation that is not itself, or is not intended to be, an active drug product or primary cosmetic product. It is understood in the art that excipients may serve multiple purposes within a formulation. It is further additionally understood that the disclosure of any excipient for any particular purpose (such as, as an emulsifier, emollient, preservative, etc.) does not restrict the use of the excipient to that particular purpose. The use of such media and agents for pharmaceutically active substances is well known in the art. Except insofar as any conventional media or agen is incompatible with the active ingredient, its use in the therapeutic compositions is contemplated. Supplementary active ingredients can also be incorporated into the compositions. In addition, various adjuvants such as are commonly used in the art can be included. These and other such compounds are described in the literature, e.g., in the Merck Index, Merck & Company, Rahway, N.J. Considerations for the inclusion of various components in pharmaceutical compositions are described, e.g., in Gilman et al. (Eds.) (1990); Goodman and Gilman's: The Pharmacological Basis of Therapeutics, 8th Ed., Pergamon Press.

The term “cosmetically acceptable carrier,” “cosmetically acceptable excipient” or “cosmetically acceptable ingredient” includes any and all solvents, diluents, emulsifiers, binders, buffers, dispersion media, coatings, antibacterial and antifungal agents, isotonic and absorption delaying agents and the like, or any other such compound as is known by those of skill in the art to be useful in preparing cosmetic compositions or formulations. It is understood in the art that excipients may serve multiple purposes within a formulation. It is further additionally understood that the disclosure of any excipient for any particular purpose (such as, as an emulsifier, emollient, preservative, etc.) does not restrict the use of the excipient to that particular purpose. The use of such media and agents for cosmetic compositions or formulations is well known in the art. Except insofar as any conventional media or agent is incompatible with the active ingredient, its use in pharmaceutical, cosmetic, or personal care compositions is contemplated. Supplementary active ingredients can also be incorporated into the compositions. In addition, various adjuvants such as are commonly used in the art can be included.

Some examples of substances, which can serve as pharmaceutically or cosmetically acceptable carriers, excipients, or ingredients, or components thereof, include but are not limited to sugars, such as lactose, glucose and sucrose; starches, such as corn starch and potato starch; cellulose and its derivatives, such as sodium carboxymethyl cellulose, ethyl cellulose, and methyl cellulose; powdered tragacanth; malt; gelatin; talc; solid lubricants, such as stearic acid and magnesium stearate; calcium sulfate; vegetable oils, such as peanut oil, soy oil, safflower oil, coconut oil, palm oil, emu oil, cottonseed oil, sesame oil, olive oil, corn oil and oil of theobroma; polyols such as propylene glycol, glycerine, sorbitol, mannitol, and polyethylene glycol; alginic acid; TWEENS; sodium lauryl sulfate; emulsifiers; wetting agents; coloring agents; flavoring agents; tableting agents, stabilizers; antioxidants; preservatives; pyrogen-free water; isotonic saline; and/or phosphate buffer solutions. Additional excipients contemplated by the present disclosure are known in the art, additional nonlimiting examples are disclosed elsewhere herein. In some embodiments, one or more components of the compositions provided herein are A.C.S. grade, reagent grade, U.S.P. grade, N.F. grade, lab grade, super refined grade, refined grade, purified grade, technical grade, or any combination thereof.

In some embodiments, the compositions of the present disclosure may comprise an oil product, which may comprise one or more of an oil, a preservative, a bulking agent, and/or a drug substance. In some embodiments, the compositions of the present disclosure may comprise soy oil, tocopherol, colloidal SiO₂, and/or a drug substance. In some embodiments, a drug substance may comprise a bacterium, lyophilized bacterium, killed bacterium, bacterial lysate, bacterial extract, bacterial growth medium, or other bacterially derived substance. In some embodiments, a drug substance may comprise a lyophile optionally containing one or more of a sugar, a sugar alcohol, a polysaccharide, an amino acid, and/or a bacterium, lyophilized bacterium, killed bacterium, bacterial lysate, bacterial extract, bacterial growth medium, or other bacterially derived substance. In some embodiments, a drug substance may comprise one or more of sorbitol, dextran, such as dextran 500, monosodium glutamate, and animal-free peptone. In some embodiments, a drug product may comprise sorbitol, dextran, such as dextran 500, monosodium glutamate, and a bacterium. In some embodiments, a drug product may comprise sorbitol, dextran, such as dextran 500, monosodium glutamate, and a bacterium of the genus Staphylococcus. In some embodiments, a drug product may comprise sorbitol, dextran, such as dextran 500, monosodium glutamate, and a bacterium of the species Staphylococcus hominis. In some embodiments, a drug product may comprise sorbitol, dextran, such as dextran 500, monosodium glutamate, and a bacterium comprising Staphylococcus hominis strain A9.

In some embodiments, a drug product may comprise a bacterium, lyophilized bacterium, killed bacterium, bacterial lysate, bacterial extract, bacterial growth medium, or other bacterially derived substance produced by fermentation. In some embodiments, a bacterium, lyophilized bacterium, killed bacterium, bacterial lysate, bacterial extract, bacterial growth medium, or other bacterially derived substance may be collected by such means as are known in the art for collecting or harvesting products of bacterial growth, such as, for example, centrifugation. In some embodiments, said bacterium, lyophilized bacterium, killed bacterium, bacterial lysate, bacterial extract, bacterial growth medium, or other bacterially derived substance may be washed and/or resuspended in a solution for administration or to facilitate lyophilization and/or further processing and/or further compounding or formulation. In some embodiments said solution may comprise or function as a cryoprotectant. In some embodiments, said solution may comprise one or more of an amino acid as disclosed herein, and/or a sugar, sugar alcohol, polymer, or a polysaccharide as disclosed herein, or any combination thereof. In some embodiments, said solution may comprise one or more of monosodium glutamate, dextran, sorbitol, mannitol, or any combination thereof. in some embodiments, said solution may comprise up to 10% (w/w) monosodium glutamate, up to 10% (w/w) dextran 500, and up to 10% (w/w) sorbitol.

In some embodiments, the compositions of the present disclosure may comprise an anhydrous lotion product, which may comprise one or more of an oil, a preservative, a bulking agent, and/or a drug substance. In some embodiments, the compositions of the present disclosure may comprise soy oil, tocopherol, colloidal SiO₂, and/or a drug substance. As noted elsewhere, in some embodiments, a drug substance may comprise a lyophile optionally containing one or more of a sugar, a sugar alcohol, a polysaccharide, an amino acid, and/or a bacterium, lyophilized bacterium, killed bacterium, bacterial lysate, bacterial extract, bacterial growth medium, or other bacterially derived substance. In some embodiments, a drug substance may comprise one or more of sorbitol, dextran, such as dextran 500, monosodium glutamate, and animal-free peptone, such as soy peptone or soy hydrolysates. In some embodiments, a drug product may comprise sorbitol, dextran, such as dextran 500, monosodium glutamate, and a bacterium. In some embodiments, a drug product may comprise sorbitol, dextran, such as dextran 500, monosodium glutamate, and a bacterium of the genus Staphylococcus. In some embodiments, a drug product may comprise sorbitol, dextran, such as dextran 500, monosodium glutamate, and a bacterium of the species Staphylococcus hominis. In some embodiments, a drug product may comprise sorbitol, dextran, such as dextran 500, monosodium glutamate, and a bacterium comprising Staphylococcus hominis strain A9. In some embodiments, an anhydrous lotion formulation may comprise one or more of a wax, a fat, a lipid, an oil, a fatty alcohol, or any combination thereof. In some embodiments, an anhydrous lotion formulation may comprise one or more of beeswax, coconut oil, shea butter, cocoa butter, lanolin, or other lipids, fats, oils, or waxes as disclosed elsewhere herein, especially those which are solid or substantially solid at a room temperature of 10° C., 15° C., 17° C., 20° C., 22° C., 25° C., 30° C., or a range defined by any of the aforementioned values, or any combination thereof. In some embodiments, an anhydrous lotion may comprise beeswax. In some embodiments, an anhydrous lotion may comprise one or more fatty alcohols as disclosed herein. In some embodiments, an anhydrous lotion may comprise one or more of lauryl, myristyl, cetyl, hexadecyl, stearyl, cetostearyl, cetearyl, isostearyl, hydroxystearyl, oleyl, ricinoleyl, behenyl, and erucyl alcohols, 2-octyl dodecanol, fatty alcohol ethers and mixtures thereof; ethoxylated fatty alcohols; ether-esters such as fatty acid esters of ethoxylated fatty alcohols and mixtures thereof, or any other fatty alcohols, fatty esters, or derivatives thereof, or any combination thereof. In some embodiments, an anhydrous lotion may comprise a microparticle or nanoparticle as described herein. In some embodiments, an anhydrous lotion may comprise a carbon, polymer, metallic, or silica microparticle or nanoparticle as disclosed herein. In some embodiments, an anhydrous lotion may comprise fumed silica.

In some embodiments, an anhydrous lotion formulation may contain 0.34% or an amount within a range from 0.05% to 1% of drug substance powder, 85.18% or an amount within a range from 45-95% of soy oil, 6.81% or an amount within a range from 1%-10% of stearyl alcohol, 6.81% or an amount within a range from 1%-10% of cetostearyl alcohol, 0.85% or an amount within a range from 0.05%-3% vitamin E, and no fumed silica.

In some embodiments, an anhydrous lotion formulation may be made by first making a drug substance powder suspended in fumed silica and soy oil with a 15% oil content, or with an oil content of between 5 and 30%, prior to addition to the remaining soy oil/fatty alcohol base lotion. In some embodiments, an anhydrous formulation may contain 0.42% or an amount within a range from 0.05%-2% of a drug substance powder, 85% or an amount within a range from 40-97% of soy oil, 7% or an amount within a range from 1%-15% stearyl alcohol, 7% or an amount within a range from 1%-15% cetostearyl alcohol, 1% or an amount within a range from 0.05%-5% vitamin E, and 0.21% or an amount within a range from 0.02%-3% fumed silica. Said suspension may be homogenous, heterogenous, or irregular.

In some embodiments, the composition (e.g., a formulation) comprises a combination of various combination groups and individual ingredients. In some embodiments, the composition comprises, consists essentially of or consists of several or all of the following groups of ingredients:

(1) a bacterial cell, strain, culture, isolate, medium, extract, lyophile, and/or a derivative thereof (e.g., a lyophilized milled and sieved powder of a coagulase negative Staphylococcus (CoNS) bacterial cell, such as Staphylococcus hominis strain A9);

(2) an oil (e.g., soy oil, sesame oil, mineral oil, corn oil, olive oil, peanut oil, macadamia nut oil, canola oil, emu oil, or any combination thereof);

(3) an inert particle (e.g., polymer nanoparticles or microparticles such as PLA, PLGA, PLA-PLGA, block copolymer, or acrylate nanoparticles or microparticles, silica, fumed silica, or amorphous silica nanoparticles or microparticles, or any combination thereof); and

(4) one or more pharmaceutically acceptable excipients (e.g., a sugar, a starch, a cellulose, powdered tragacanth, malt, gelatin, talc, a solid lubricant, calcium sulfate, a vegetable oil, a polyol, alginic acid; a TWEEN, sodium lauryl sulfate, an emulsifier, a wetting agent, a coloring agent, a flavoring agent, a tableting agent, a stabilizer; an antioxidant, a preservative, pyrogen-free water, isotonic saline, a phosphate buffer solution, or any combination thereof).

In some embodiments, group (1), group (2), group (3), and group (4) above are provided in a range of about 0.001%-99% (e.g., 0.001%, 0.01%, 0.1%, 0.2%, 0.3%, 0.4%, 0.5%, 0.6%, 0.7%, 0.8%, 0.9%, 1.0%, 1.1%, 1.2%, 1.3%, 1.4%, 1.5%, 1.6%, 1.7%, 1.8%, 1.9%, 2.0%, 2.1%, 2.2%, 2.3%, 2.4%, 2.5%, 2.6%, 2.7%, 2.8%, 2.9%, 3.0%, 3.1%, 3.2%, 3.3%, 3.4%, 3.5%, 3.6%, 3.7%, 3.8%, 3.9%, 4.0%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or ranges in between) of the total composition. By way of example, if soy oil is provided at 80% vis-a-vis the select group of ingredients listed in group (2), and group (2) is provided as 60% of the total composition, then soy oil will be present as 48% of the total composition. The percentages provided above for the groups (1)-(4) are provided as % m/m in some embodiments. In other embodiments, these ingredients are provided as % w/w, % m/v, % v/v, % m/w, or % w/v. In several embodiments, a therapeutic or effective amount of each ingredient is included in the composition. A therapeutic or effective amount may be that which reduces bacterial infection, reduces bacterial overgrowth, and/or reduces dysbiosis.

The compositions described herein are preferably provided in a unit dosage form. As used herein, a “unit dosage form” is a composition containing an amount of a compound that is suitable for administration to a subject, in a single dose, according to good medical practice. The preparation of a single or unit dosage form however, does not imply that the dosage form is administered once per day or once per course of therapy. A unit dosage form may comprise, consist essentially of, or consist of a single daily dose or a fractional sub-dose wherein several unit dosage forms are to be administered over the course of a day in order to complete a daily dose. According to the present disclosure, a unit dosage form can be given more or less often that once daily, and can be administered more than once during a course of therapy. Such dosage forms can be administered in any manner consistent with their formulation, including orally, rectally, nasally, and/or parenterally. While single administrations are specifically contemplated, the compositions administered according to the methods described herein may also be administered as a continuous infusion or via an implantable infusion pump.

The methods as described herein may utilize any of a variety of suitable forms for a variety of routes for administration, for example, for topical, oral, mucosal, nasal, or rectal routes of administration. Depending upon the particular route of administration desired, a variety of pharmaceutically-acceptable carriers well-known in the art can be used. Pharmaceutically-acceptable carriers include, for example, solid or liquid fillers, diluents, hydrotropes, surface-active agents, and encapsulating substances. Optional pharmaceutically-active materials can be included, which do not substantially interfere with the activity of the one or more compounds in the formulation. The amount of carrier employed in conjunction with the compound is sufficient to provide a practical quantity of material for administration per unit dose of the compound. Techniques and compositions for making dosage forms useful in the methods described herein are described in the following references, all incorporated by reference herein: Modern Pharmaceutics, 4th Ed., Chapters 9 and 10 (Banker & Rhodes, editors, 2002); Lieberman et al., Pharmaceutical Dosage Forms: Tablets (1989); and Ansel, Introduction to Pharmaceutical Dosage Forms 8th Edition (2004).

In some embodiments, the excipients can include a topical pharmaceutically- and cosmetically-acceptable emollient. As used herein, “emollients” refer to materials used for the prevention or relief of dryness, as well as for the protection of the skin. Sagarin, Cosmetics, Science and Technology, 2nd Edition, Vol. 1, pp. 32-43 (1972), which is incorporated herein by reference in its entirety, contains numerous examples of suitable materials for use as emollients.

The actual unit dose of the compositions described herein depends on the one or more compounds in the formulation. In some embodiments, the amount of each compound in the formulation can be from 5 mg/kg to 500 mg/kg or more of body weight per day, from 10 mg/kg or less to 70 mg/kg, from 50 mg/kg to 80 mg/kg of body weight per day, from 70 mg/kg to 120 mg/kg of body weight per day, from 100 mg/kg to 300 mg/kg of body weight per day, or from 250 mg/kg to 500 mg/kg of body weight per day. In some embodiments, the dose can be less than 100 mg/kg, 500 mg/kg, 300 mg/kg, 200 mg/kg, 150 mg/kg, 100 mg/kg, 50 mg/kg, 40 mg/kg, 30 mg/kg, 25 mg/kg, 20 mg/kg, 10 mg/kg, 7.5 mg/kg, 6 mg/kg, 5 mg/kg, 4 mg/kg, 3 mg/kg, 2.5 mg/kg, or 1 mg/kg of body weight per day or an amount that is within a range defined by any two of the aforementioned amounts. In some embodiments, the actual unit dose is 5, 10, 25, 50, 75, 100, 150, or 200 mg/kg of body weight per day or an amount that is within a range defined by any two of the aforementioned amounts. Thus, for administration to a 70 kg person, for example, the dosage range is from 350 mg to 750 mg, from 500 mg to 1 g, from 750 mg to 2 g, from 1 g to 5 g, from 2.5 g to 6 g, from 4 g to 10 g, from 8 g to 20 g, from 15 g to 35 g, or from 1 g or less to 35 g or more, or an amount that is within a range defined by any two of the aforementioned amounts. In some embodiments, the actual unit dose is 6 g. In some embodiments the actual unit dose is 10 g. In some embodiments, the actual unit dose is 35 g. In some embodiments, the actual unit dose is 1 g or less but not zero. In some embodiments, the actual unit dose is 10 g or less but not zero. In some embodiments, the actual unit dose is 35 mg or less but not zero.

In some embodiments, the actual dose administered may be 0.002 g/cm² or less, 0.02 g/cm² or less, 0.2 g/cm² or less, 2 g/cm² or less, or more than 2 g/cm². In some embodiments, the actual dose administered may be 0.005 ml/cm²or less, 0.05 ml/cm² or less, 0.5 ml/cm² or less, 5 ml/cm² or less, or more than 5 ml/cm². In some embodiments, the actual dose administered may be 1 CFU/cm² or less, 10 CFU/cm² or less, 10² CFU/cm² or less, 10³ CFU/cm² or less, 10³ CFU/cm² or less, 10⁵ CFU/cm² or less, 10⁶ CFU/cm² or less, 10⁷ CFU/cm² or less, 10⁸ CFU/cm²or less, 10⁹ CFU/cm²or less, 10¹⁰ CFU/cm²or less, or more than 10¹⁰ CFU/cm². In some embodiments, the actual dose administered may be 2×10⁵ CFU/cm² or less. In some embodiments, the actual dose administered may be more than 2×10⁵ CFU/cm² or less.

The phrase “loading dose,” as used herein refers to an initial dose of a compound which is higher than subsequent doses.

The phrase “maintenance dose,” as used herein refers to a subsequent dose that follows a loading dose, and occurs later in time than a loading dose. One of ordinary skill in the art will be aware that the dosage form or mode of administration of a maintenance dose can be different from that used for the loading dose. In any of the embodiments disclosed herein, a maintenance dose may comprise administration of the unit dosage form on any dosing schedule contemplated herein, including but not limited to, monthly or multiple times per month, biweekly or multiple times each two weeks, weekly or multiple times per week, daily or multiple times per day. It is contemplated within the present disclosure that dosing holidays can be incorporated into the dosing period of the maintenance dose. Such dosing holidays may occur immediately after the administration of the loading dose or at any time during the period of administration of the maintenance dose. As used herein, the period of administration of the maintenance dose can be referred to as the “maintenance phase” of the treatment period.

The phrase “mode of administration” as used herein refers to the avenue by which one or more compounds are administered to a subject. As used herein, “mode of administration” comprises the dosage form (for example, a tablet, powder, dissolved liquid, suspension, emulsion, lotion, oil, etc.) and mechanism by which the dosage form is applied to the subject (for example, topically, such as by metered pump, resorbable film, nonresorbable film, patch, a wipe, such as a cloth, paper, or fiber wipe, drops, massage, lavage, rinse, immersion, or other methods as are known in the art for the administration of topical or other compositions to a subject). As used herein, “mode of administration” also comprises the dose, dose amount, and dosing schedule by which a compound is administered to a subject.

In some embodiments, the compositions to be administered according to the methods of the present disclosure are provided with, or mixed into, a foodstuff, beverage, or other ingestible item. In some embodiments, said beverage, foodstuff, or other ingestible item may comprise, consist essentially of, or consist of one or more of a candy, an applesauce, a yogurt, a soft pudding, a gelatin foodstuff, a juice, milk, a soy or nut beverage, a thickened beverage, or a cheese, or any combination thereof. One of ordinary skill will readily recognize that the combination of the compositions to be administered according to the methods of the disclosure can be combined with any suitable food or beverage to facilitate ingestion of the compositions.

Because levels of some microbial strains or species will be expected to fluctuate in response to external and/or somatically derived stimuli, the methods according to the present disclosure contemplate varying or controlling the timing and/or the frequency of administration of the compositions described herein, in order to enhance the effectiveness of the treatment, for example, by optimizing the timing of the emplacement of one or more microbial strains or extracts, in such a manner as to maintain both the somatic and the microbial health of the subject. In some embodiments, the compositions to be administered according to the methods of the present disclosure can be administered before, during, or after cleaning or rinsing of the skin, and/or before during or after additional treatments of the skin including moisturization, disinfection, administration or coadministration of additional therapeutics or cosmetics, before, during or after application of makeup, perfume, or deodorant, before, during, or after shaving, waxing, or exfoliation. In some embodiments, the compositions to be administered according to the methods of the present disclosure can be administered within 1-5 minutes, within 3-10 minutes, within 6-15 minutes, within 10-20 minutes, within 15-30 minutes, within 20-45 minutes, or within one hour before or after washing, rinsing, disinfection, exfoliation or other activity contacting the affected area of the skin. In some embodiments, the compositions to be administered according to the methods of the present disclosure can be administered without washing, rinsing, disinfection, exfoliation or other activity contacting the affected area of the skin, such as between 1-3 hours, between 2-5 hours, between 4-8 hours, between 6-12 hours, between 9-18 hours, between 12-24 hours, or more than 24 hours before or after washing, rinsing, disinfection, exfoliation or other activity contacting the affected area of the skin. In some embodiments, the compositions of the present disclosure may be administered concurrently with a meal or other ingestion of a foodstuff, or concurrently with a beverage or prescribed drink. In some further embodiments, the compositions to be administered according to the methods of the present disclosure can be administered immediately before or immediately after a meal or other ingestion of a foodstuff. In some further embodiments, the compositions to be administered according to the methods of the present disclosure can be administered within 1-5 minutes, within 3-10 minutes, within 6-15 minutes, within 10-20 minutes, within 15-30 minutes, within 20-45 minutes, within one hour, or within two hours before or after a meal or other ingestion of a foodstuff. In some embodiments, the compositions to be administered according to the methods of the present disclosure can be administered between meals or in the absence of the ingestion of a meal, beverage, or other foodstuff such as between 1-3 hours, between 2-5 hours, between 4-8 hours, between 6-12 hours, between 9-18 hours, between 12-24 hours, or more than 24 hours before or after ingestion of a meal, beverage, or other foodstuff.

As used herein, “duration of the treatment” refers to the time commencing with administration of the first dose and concluding with the administration of the final dose, such length of time being determined by one of ordinary skill in the art of treating skin disorders or disorders implicating dysbiosis of the skin, with reference to the symptoms and health of the subject being treated therefor. Such duration can be determined with reference to periodic, sporadic, or ongoing monitoring of the appearance of the skin, of metabolic genetic markers of inflammation, of the presence or absence of particular commensal bacteria and/or pathogens, or by any other method as is known to one of skill in the art of treating skin disorders or disorders implicating dysbiosis of the skin.

As used herein, “dosing holiday” refers to a period of 24 hours or more during which either no dose is administered to the subject, or a reduced dose is administered to the subject. As used herein, “reduced dose” refers to a dose that is less than the total daily dose to be administered to a subject.

Topical formulations may generally comprise one or more of a pharmaceutical carrier, co-solvent, emulsifier, penetration enhancer, preservative system, carrier, protectant, cryoprotectant, vehicle, lubricant, diluent, abrasive, and/or an emollient and/or other excipient or excipients. Examples of compounds in the classes of waxes, oils, polymers, fatty acids, and the like that are useful as carriers, protectants, cryoprotectants, vehicles, lubricants, emollients, diluents, or other excipients include, but are not limited to, hydrocarbon oils and waxes such as mineral oil, petrolatum, paraffin, ceresin, ozokerite, microcrystalline wax, polyethylene, and perhydrosqualene; silicone oil, such as dimethyl polysiloxanes, methylphenyl polysiloxanes, and water-soluble and alcohol-soluble silicone glycol copolymers. Other suitable compounds include triglyceride esters such as vegetable and animal fats and oils including castor oil, safflower oil, cotton seed oil, corn oil, olive oil, cod liver oil, almond oil, avocado oil, palm oil, sesame oil, macadamia nut oil, jojoba oil, emu oil, and soybean oil; acetoglyceride esters, such as acetylated monoglycerides; ethoxylated glycerides, such as ethoxylated glycerylmonostearate; alkyl esters of fatty acids including methyl, isopropyl, and butyl esters of fatty acids, alkyl esters including hexyl laurate, isohexyl laurate, iso-hexyl palmitate, isopropyl palmitate, decyl oleate, isodecyl oleate, hexadecyl stearate, decyl stearate, isopropyl isostearate, diisopropyl adipate, dissohexyl adipate, di-hexyldecyl adipate, diisopropyl sebacate, lauryl lactate, myristyl lactate, and cetyl lactate; and alkenyl esters of fatty acids such as oleyl myristate, oleyl stearate, and oleyl oleate. Other suitable classes of compounds useful for the formulation of the present compositions include fatty acids such as pelargonic, lauric, myristic, palmitic, stearic, isostearic, hydroxystearic, oleic, linoleic, ricinoleic, arachidic, behenic, and erucic acids; fatty alcohols such as lauryl, myristyl, cetyl, hexadecyl, stearyl, cetostearyl, cetearyl, isostearyl, hydroxystearyl, oleyl, ricinoleyl, behenyl, and erucyl alcohols, as well as 2-octyl dodecanol and mixtures thereof; fatty alcohol ethers and mixtures thereof; ethoxylated fatty alcohols; ether-esters such as fatty acid esters of ethoxylated fatty alcohols and mixtures thereof; lanolin and derivatives including lanolin oil, lanolin wax, lanolin alcohols, lanolin fatty acids, isopropyllanolate, ethoxylated lanolin, ethoxylated lanolin alcohols, ethoxolated cholesterol, propoxylated lanolin alcohols, acetylated lanolin, acetylated lanolin alcohols, lanolin alcohols linoleate, lanolin alcohols recinoleate, acetate of lanolin alcohols recinoleate, acetate of lanolin alcohols recinoleate, acetate of ethoxylated alcohols esters, products of hydrogenolysis of lanolin, ethoxylated hydrogenated lanolin, ethoxylated sorbitol lanolin, and liquid and semisolid lanolin absorption bases (ethoxylated hydrogenated lanolin, ethoxylated sorbitol lanolin, and liquid and semisolid lanolin absorption bases are illustrative of emollients or topical excipients derived from lanolin); polyhydric alcohols and polyether derivatives such as propylene glycol, dipropylene glycol, polypropylene glycols, polyoxyethylene polyoxypropylene glycols, polyoxypropylene polyoxyethylene glycols, glycerol, sorbitol, ethoxylated sorbitol, hydroxypropylsorbitol, polyethylene glycols, methoxy polyethylene glycols, polyalkylene glycols and derivatives, hexylene glycol(2-methyl-2,4-pentanediol), 1,3-butylene glycol, 1,2,6-hexanetriol, 2-ethyl,3-hexanediol, and polyoxypropylene derivatives of trimethylolpropane; polydydric alcohol esters such as ethylene glycol mono- and di-fatty acid esters, diethylene glycol mono- and di-fatty acid esters, polyethylene glycol, mono- and di-fatty acid esters, propylene glycol mono- and di-fatty esters, polypropylene glycol monooleate, polypropylene glycol monostearate, ethoxylatedpropylene glycol monostearate, glyceryl mono- and di-fatty acid esters, polyglycerol poly-fatty acid esters, ethoxylated glyceryl monostearate, 1,3-butylene glycolmonostearate, 1,3-butylene glycol distearate, polyoxyethylene polyol fatty acid ester, sorbitan fatty acid esters, and polyoxyethylene sorbitan fatty acid esters; wax esters such as beeswax, spermaceti, myristyl myristate and stearyl stearate; beeswax derivatives, e.g., polyoxyethylene sorbitol beeswax; vegetable waxes including carnauba and candelilla waxes; and phospholipids, such as lecithin and derivatives; sterols including, for example, cholesterol and cholesterol fatty acid esters; amides such as fatty acid amides, ethoxylated fatty acid amides and solid fatty acid alkanolamides. In some embodiments, the compositions and methods of the present disclosure contemplate mixtures incorporating any of the foregoing. In some embodiments, the composition may comprise one or more compounds selected from the group consisting of glycerol, hexanetriol, butanetriol, lactic acid, urea, pyrrolidone carboxylic acid, amino acids, guanidine, diglycerol and triglycerol. In a typical embodiment, the compositions as disclosed herein can include sesame or soy oil. In some embodiments, the compositions as disclosed herein comprise any or any combination of the foregoing. In some embodiments, the oils are highly refined or super-refined. In some embodiments, the oils are substantially free of allergens or detectable protein or polysaccharide contaminants.

In some embodiments, the excipients can include fatty acid triglycerides, namely the triglyceryl esters of saturated and/or unsaturated, branched and/or unbranched alkanecarboxylic acids having a chain length of from 6 to 24 carbon atoms, in particular 6-10 carbon atoms. In some embodiments, the excipients can include fatty acid diglycerides, namely the diglycerol esters of saturated and/or unsaturated, branched and/or unbranched alkanecarboxylic acids having a chain length of from 6 to 24 carbon atoms, in particular 6-10 carbon atoms. In some embodiments, the excipients can include fatty acid monoglycerides, namely the monoglycerol esters of saturated and/or unsaturated, branched and/or unbranched alkanecarboxylic acids having a chain length of from 6 to 24 carbon atoms, in particular 6-10 carbon atoms.

In some embodiments, the topical composition disclosed herein can comprise at least 0.1% (w/w), 0.2% (w/w), 0.3% (w/w), 0.4% (w/w), 0.5% (w/w), 0.6% (w/w), 0.7% (w/w), 0.8% (w/w), 0.9% (w/w), 1.0% (w/w), 1.1% (w/w), 1.2% (w/w), 1.3% (w/w), 1.4% (w/w), 1.5% (w/w), 1.6% (w/w), 1.7% (w/w), 1.8% (w/w), 1.9% (w/w), 2.0% (w/w), 2.1% (w/w), 2.2% (w/w), 2.3% (w/w), 2.4% (w/w), 2.5% (w/w), 2.6% (w/w), 2.7% (w/w), 2.8% (w/w), 2.9% (w/w), 3.0% (w/w), 3.1% (w/w), 3.2% (w/w), 3.3% (w/w), 3.4% (w/w), 3.5% (w/w), 3.6% (w/w), 3.7% (w/w), 3.8% (w/w), 3.9% (w/w), or 4.0% (w/w) of an oil, wax, fatty acid, fatty ester, lipid, triglyceride, or derivatives or equivalents thereof which may function as a base, carrier, emollient, or other excipient. In some embodiments, the topical composition disclosed herein can comprise 0.1% (w/w), 0.2% (w/w), 0.3% (w/w), 0.4% (w/w), 0.5% (w/w), 0.6% (w/w), 0.7% (w/w), 0.8% (w/w), 0.9% (w/w), 1.0% (w/w), 1.1% (w/w), 1.2% (w/w), 1.3% (w/w), 1.4% (w/w), 1.5% (w/w), 1.6% (w/w), 1.7% (w/w), 1.8% (w/w), 1.9% (w/w), 2.0% (w/w), 2.1% (w/w), 2.2% (w/w), 2.3% (w/w), 2.4% (w/w), 2.5% (w/w), 2.6% (w/w), 2.7% (w/w), 2.8% (w/w), 2.9% (w/w), 3.0% (w/w), 3.1% (w/w), 3.2% (w/w), 3.3% (w/w), 3.4% (w/w), 3.5% (w/w), 3.6% (w/w), 3.7% (w/w), 3.8% (w/w), 3.9% (w/w), 4% (w/w), 5% (w/w), 6% (w/w), 7% (w/w), 8% (w/w), 9% (w/w), 10% (w/w), 20% (w/w) or 30% (w/w) of an oil, wax, fatty acid, fatty ester, lipid, triglyceride, or derivatives or equivalents thereof which may function as a base, carrier, emollient, or other excipient, or a range defined by any two of the preceding values. In some embodiments, the compositions disclosed herein may comprise 0.001% (w/w), 1% (w/w), 2% (w/w), 3% (w/w), 4% (w/w), 5% (w/w), 6% (w/w), 7% (w/w), 8% (w/w), 9% (w/w), 10% (w/w), 11% (w/w), 12% (w/w), 13% (w/w), 14% (w/w), 15% (w/w), 16% (w/w), 17% (w/w), 18% (w/w), 19% (w/w), 20% (w/w), 21% (w/w), 22% (w/w), 23% (w/w), 24% (w/w), 25% (w/w), 26% (w/w), 27% (w/w), 28% (w/w), 29% (w/w), 30% (w/w), 31% (w/w), 32% (w/w), 33% (w/w), 34% (w/w), 35% (w/w), 36% (w/w), 37% (w/w), 38% (w/w), 39% (w/w), 40% (w/w), 41% (w/w), 42% (w/w), 43% (w/w), 44% (w/w), 45% (w/w), 46% (w/w), 47% (w/w), 48% (w/w), 49% (w/w), 50% (w/w), 51% (w/w), 52% (w/w), 53% (w/w), 54% (w/w), 55% (w/w), 56% (w/w), 57% (w/w), 58% (w/w), 59% (w/w), 60% (w/w), 61% (w/w), 62% (w/w), 63% (w/w), 64% (w/w), 65% (w/w), 66% (w/w), 67% (w/w), 68% (w/w), 69% (w/w), 70% (w/w), 71% (w/w), 72% (w/w), 73% (w/w), 74% (w/w), 75% (w/w), 76% (w/w), 77% (w/w), 78% (w/w), 79% (w/w), 80% (w/w), 81% (w/w), 82% (w/w), 83% (w/w), 84% (w/w), 85% (w/w), 86% (w/w), 87% (w/w), 88% (w/w), 89% (w/w), 90% (w/w), 91% (w/w), 92% (w/w), 93% (w/w), 94% (w/w), 95% (w/w), 96% (w/w), 97% (w/w), 98% (w/w), 99% (w/w), or more of an oil, wax, fatty acid, fatty ester, lipid, triglyceride, or derivatives or equivalents thereof which may function as a base, carrier, emollient, or other excipient, or a range defined by any two of the preceding values.

In some embodiments, the topical composition disclosed herein can comprise at least 0.1% (w/w), 0.2% (w/w), 0.3% (w/w), 0.4% (w/w), 0.5% (w/w), 0.6% (w/w), 0.7% (w/w), 0.8% (w/w), 0.9% (w/w), 1.0% (w/w), 1.1% (w/w), 1.2% (w/w), 1.3% (w/w), 1.4% (w/w), 1.5% (w/w), 1.6% (w/w), 1.7% (w/w), 1.8% (w/w), 1.9% (w/w), 2.0% (w/w), 2.1% (w/w), 2.2% (w/w), 2.3% (w/w), 2.4% (w/w), 2.5% (w/w), 2.6% (w/w), 2.7% (w/w), 2.8% (w/w), 2.9% (w/w), 3.0% (w/w), 3.1% (w/w), 3.2% (w/w), 3.3% (w/w), 3.4% (w/w), 3.5% (w/w), 3.6% (w/w), 3.7% (w/w), 3.8% (w/w), 3.9% (w/w), or 4.0% (w/w) of an emollient. In some embodiments, the topical composition disclosed herein can comprise 0.1% (w/w), 0.2% (w/w), 0.3% (w/w), 0.4% (w/w), 0.5% (w/w), 0.6% (w/w), 0.7% (w/w), 0.8% (w/w), 0.9% (w/w), 1.0% (w/w), 1.1% (w/w), 1.2% (w/w), 1.3% (w/w), 1.4% (w/w), 1.5% (w/w), 1.6% (w/w), 1.7% (w/w), 1.8% (w/w), 1.9% (w/w), 2.0% (w/w), 2.1% (w/w), 2.2% (w/w), 2.3% (w/w), 2.4% (w/w), 2.5% (w/w), 2.6% (w/w), 2.7% (w/w), 2.8% (w/w), 2.9% (w/w), 3.0% (w/w), 3.1% (w/w), 3.2% (w/w), 3.3% (w/w), 3.4% (w/w), 3.5% (w/w), 3.6% (w/w), 3.7% (w/w), 3.8% (w/w), 3.9% (w/w), 4% (w/w), 5% (w/w), 6% (w/w), 7% (w/w), 8% (w/w), 9% (w/w), 10% (w/w), 20% (w/w) or 30% (w/w) of an emollient or a range defined by any two of the preceding values. In some embodiments, the compositions disclosed herein may comprise 0.001% (w/w), 1% (w/w), 2% (w/w), 3% (w/w), 4% (w/w), 5% (w/w), 6% (w/w), 7% (w/w), 8% (w/w), 9% (w/w), 10% (w/w), 11% (w/w), 12% (w/w), 13% (w/w), 14% (w/w), 15% (w/w), 16% (w/w), 17% (w/w), 18% (w/w), 19% (w/w), 20% (w/w), 21% (w/w), 22% (w/w), 23% (w/w), 24% (w/w), 25% (w/w), 26% (w/w), 27% (w/w), 28% (w/w), 29% (w/w), 30% (w/w), 31% (w/w), 32% (w/w), 33% (w/w), 34% (w/w), 35% (w/w), 36% (w/w), 37% (w/w), 38% (w/w), 39% (w/w), 40% (w/w), 41% (w/w), 42% (w/w), 43% (w/w), 44% (w/w), 45% (w/w), 46% (w/w), 47% (w/w), 48% (w/w), 49% (w/w), 50% (w/w), 51% (w/w), 52% (w/w), 53% (w/w), 54% (w/w), 55% (w/w), 56% (w/w), 57% (w/w), 58% (w/w), 59% (w/w), 60% (w/w), 61% (w/w), 62% (w/w), 63% (w/w), 64% (w/w), 65% (w/w), 66% (w/w), 67% (w/w), 68% (w/w), 69% (w/w), 70% (w/w), 71% (w/w), 72% (w/w), 73% (w/w), 74% (w/w), 75% (w/w), 76% (w/w), 77% (w/w), 78% (w/w), 79% (w/w), 80% (w/w), 81% (w/w), 82% (w/w), 83% (w/w), 84% (w/w), 85% (w/w), 86% (w/w), 87% (w/w), 88% (w/w), 89% (w/w), 90% (w/w), 91% (w/w), 92% (w/w), 93% (w/w), 94% (w/w), 95% (w/w), 96% (w/w), 97% (w/w), 98% (w/w), 99% (w/w), or 100% (w/w) of an emollient or a range defined by any two of the preceding values.

In some embodiments, the topical composition disclosed herein can comprise at least 0.1% (w/w), 0.2% (w/w), 0.3% (w/w), 0.4% (w/w), 0.5% (w/w), 0.6% (w/w), 0.7% (w/w), 0.8% (w/w), 0.9% (w/w), 1.0% (w/w), 1.1% (w/w), 1.2% (w/w), 1.3% (w/w), 1.4% (w/w), 1.5% (w/w), 1.6% (w/w), 1.7% (w/w), 1.8% (w/w), 1.9% (w/w), 2.0% (w/w), 2.1% (w/w), 2.2% (w/w), 2.3% (w/w), 2.4% (w/w), 2.5% (w/w), 2.6% (w/w), 2.7% (w/w), 2.8% (w/w), 2.9% (w/w), 3.0% (w/w), 3.1% (w/w), 3.2% (w/w), 3.3% (w/w), 3.4% (w/w), 3.5% (w/w), 3.6% (w/w), 3.7% (w/w), 3.8% (w/w), 3.9% (w/w), or 4.0% (w/w) of glycerol. In some embodiments, the topical composition disclosed herein can comprise 0.1% (w/w), 0.2% (w/w), 0.3% (w/w), 0.4% (w/w), 0.5% (w/w), 0.6% (w/w), 0.7% (w/w), 0.8% (w/w), 0.9% (w/w), 1.0% (w/w), 1.1% (w/w), 1.2% (w/w), 1.3% (w/w), 1.4% (w/w), 1.5% (w/w), 1.6% (w/w), 1.7% (w/w), 1.8% (w/w), 1.9% (w/w), 2.0% (w/w), 2.1% (w/w), 2.2% (w/w), 2.3% (w/w), 2.4% (w/w), 2.5% (w/w), 2.6% (w/w), 2.7% (w/w), 2.8% (w/w), 2.9% (w/w), 3.0% (w/w), 3.1% (w/w), 3.2% (w/w), 3.3% (w/w), 3.4% (w/w), 3.5% (w/w), 3.6% (w/w), 3.7% (w/w), 3.8% (w/w), 3.9% (w/w), 4% (w/w), 5% (w/w), 6% (w/w), 7% (w/w), 8% (w/w), 9% (w/w), 10% (w/w), 20% (w/w) or 30% (w/w) of a preservative or a range defined by any two of the preceding values.

In some embodiments, a composition as disclosed herein can comprise at least 0.1% (w/w), 0.2% (w/w), 0.3% (w/w), 0.4% (w/w), 0.5% (w/w), 0.6% (w/w), 0.7% (w/w), 0.8% (w/w), 0.9% (w/w), 1.0% (w/w), 1.1% (w/w), or 1.2% (w/w) of a fatty acid monoglyceride. In some embodiments, the topical composition disclosed herein can comprise 0.1% (w/w), 0.2% (w/w), 0.3% (w/w), 0.4% (w/w), 0.5% (w/w), 0.6% (w/w), 0.7% (w/w), 0.8% (w/w), 0.9% (w/w), 1.0% (w/w), 1.1% (w/w), 1.2% (w/w), 1.5% (w/w), 2% (w/w), 3% (w/w), 4% (w/w), 5% (w/w), 6% (w/w), 7% (w/w), 8% (w/w), 9% (w/w), 10% (w/w), 20% (w/w) or 30% (w/w) of a fatty acid monoglyceride or a range defined by any two of the preceding values.

In some embodiments, a composition as disclosed herein can comprise at least 0.1% (w/w), 0.2% (w/w), 0.3% (w/w), 0.4% (w/w), 0.5% (w/w), 0.6% (w/w), 0.7% (w/w), 0.8% (w/w), 0.9% (w/w), 1.0% (w/w), 1.1% (w/w), or 1.2% (w/w) of a fatty acid diglyceride. In some embodiments, the topical composition disclosed herein can comprise 0.1% (w/w), 0.2% (w/w), 0.3% (w/w), 0.4% (w/w), 0.5% (w/w), 0.6% (w/w), 0.7% (w/w), 0.8% (w/w), 0.9% (w/w), 1.0% (w/w), 1.1% (w/w), 1.2% (w/w), 1.5% (w/w), 2% (w/w), 3% (w/w), 4% (w/w), 5% (w/w), 6% (w/w), 7% (w/w), 8% (w/w), 9% (w/w), 10% (w/w), 20% (w/w) or 30% (w/w) of a fatty acid diglyceride or a range defined by any two of the preceding values.

In some embodiments, a composition as disclosed herein can comprise at least 0.1% (w/w), 0.2% (w/w), 0.3% (w/w), 0.4% (w/w), 0.5% (w/w), 0.6% (w/w), 0.7% (w/w), 0.8% (w/w), 0.9% (w/w), 1.0% (w/w), 1.1% (w/w), or 1.2% (w/w) of a fatty acid triglyceride. In some embodiments, the topical composition disclosed herein can comprise 0.1% (w/w), 0.2% (w/w), 0.3% (w/w), 0.4% (w/w), 0.5% (w/w), 0.6% (w/w), 0.7% (w/w), 0.8% (w/w), 0.9% (w/w), 1.0% (w/w), 1.1% (w/w), 1.2% (w/w), 1.5% (w/w), 2% (w/w), 3% (w/w), 4% (w/w), 5% (w/w), 6% (w/w), 7% (w/w), 8% (w/w), 9% (w/w), 10% (w/w), 20% (w/w) or 30% (w/w) of a fatty acid triglyceride or a range defined by any two of the preceding values.

Further examples of suitable emollients may include, in some embodiments, amino acids, chondroitin sulfate, diglycerin, erythritol, fructose, glucose, glycerin, glycerol polymers, glycol, 1,2,6-hexanetriol, honey, hyaluronic acid, hydrogenated honey, hydrogenated starch hydrolysate, inositol, lactitol, maltitol, maltose, mannitol, natural moisturizing factor, PEG-15 butanediol, polyglyceryl sorbitol, salts of pyrollidone carboxylic acid, potassium PCA, propylene glycol, sodium glucuronate, sodium PCA, sorbitol, sucrose, trehalose, urea, and xylitol. Other examples of moisturizing agents which may be incorporated into the compositions disclosed herein may include one or more of acetylated lanolin, acetylated lanolin alcohol, acrylates/C10-30 alkyl acrylate crosspolymer, acrylates copolymer, alanine, algae extract, aloe barbadensis, aloe-barbadensis extract, aloe barbadensis gel, althea officinalis extract, aluminum starch octenylsuccinate, aluminum stearate, apricot (prunus armeniaca) kernel oil, arginine, arginine aspartate, arnica montana extract, ascorbic acid, ascorbyl palmitate, aspartic acid, avocado (persea gratissima) oil, barium sulfate, barrier sphingolipids, butyl alcohol, beeswax, behenyl alcohol, beta-sitosterol, BHT, birch (betula alba) bark extract, borage (borago officinalis) extract, 2-bromo-2-nitropropane-1,3-diol, butcherbroom (ruscus aculeatus) extract, butylene glycol, calendula officinalis extract, calendula officinalis oil, candelilla (euphorbia cerifera) wax, canola oil, caprylic/capric triglyceride, cardamon (elettaria cardamomum) oil, carnauba (copernicia cerifera) wax, carrageenan (chondrus crispus), carrot (daucus carota sativa) oil, castor (ricinus communis) oil, ceramides, ceresin, ceteareth-5, ceteareth-12, ceteareth-20, cetearyl octanoate, ceteth-20, ceteth-24, cetyl acetate, cetyl octanoate, cetyl palmitate, chamomile (anthemis nobilis) oil, cholesterol, cholesterol esters, cholesteryl hydroxystearate, citric acid, clary (salvia sclarea) oil, cocoa (theobroma cacao) butter, coco-caprylate/caprate, coconut (cocos nucifera) oil, collagen, collagen amino acids, corn (zea mays) oil, fatty acids, decyl oleate, dextrin, diazolidinyl urea, dimethicone copolyol, dimethiconol, dioctyl adipate, dioctyl succinate, dipentaerythrityl hexacaprylate/hexacaprate, DMDM hydantoin, DNA, erythritol, ethoxydiglycol, ethyl linoleate, eucalyptus globulus oil, evening primrose (oenothera biennis) oil, fatty acids, tructose, gelatin, geranium maculatum oil, glucosamine, glucose glutamate, glutamic acid, glycereth-26, glycerin, glycerol, glyceryl distearate, glyceryl hydroxystearate, glyceryl laurate, glyceryl linoleate, glyceryl myristate, glyceryl oleate, glyceryl stearate, glyceryl stearate SE, glycine, glycol stearate, glycol stearate SE, glycosaminoglycans, grape (vitis vinifera) seed oil, hazel (corylus americana) nut oil, hazel (corylus avellana) nut oil, hexylene glycol, honey, hyaluronic acid, hybrid safflower (carthamus tinctorius) oil, hydrogenated castor oil, hydrogenated coco-glycerides, hydrogenated coconut oil, hydrogenated lanolin, hydrogenated lecithin, hydrogenated palm glyceride, hydrogenated palm kernel oil, soybean oil, safflower oil, olive oil, sesame oil, hydrogenated soybean oil, hydrogenated tallow glyceride, hydrogenated vegetable oil, hydrolyzed collagen, hydrolyzed elastin, hydrolyzed glycosaminoglycans, hydrolyzed keratin, hydrolyzed soy protein, hydroxylated lanolin, hydroxyproline, imidazolidinyl urea, iodopropynyl butylcarbamate, isocetyl stearate, isocetyl stearoyl stearate, isodecyl oleate, isopropyl isostearate, isopropyl lanolate, isopropyl myristate, isopropyl palmitate, isopropyl stearate, isostearamide DEA, isostearic acid, isostearyl lactate, isostearyl neopentanoate, jasmine (jasminum officinale) oil, jojoba (buxus chinensis) oil, kelp, kukui (aleurites moluccana) nut oil, lactamide MEA, laneth-16, laneth-10 acetate, lanolin, lanolin acid, lanolin alcohol, lanolin oil, lanolin wax, lavender (lavandula angustifolia) oil, lecithin, lemon (citrus medica limonum) oil, linoleic acid, linolenic acid, macadamia ternifolia nut oil, magnesium stearate, magnesium sulfate, maltitol, matricaria (chamomilla recutita) oil, methyl glucose sesquistearate, methylsilanol PCA, microcrystalline wax, mineral oil, mink oil, mortierella oil, myristyl lactate, myristyl myristate, myristyl propionate, neopentyl glycol dicaprylate/dicaprate, octyldodecanol, octyldodecyl myristate, octyldodecyl stearoyl stearate, octyl hydroxystearate, octyl palmitate, octyl salicylate, octyl stearate, oleic acid, olive (olea europaea) oil, orange (citrus aurantium dulcis) oil, palm (elaeis guineensis) oil, palmitic acid, pantethine, panthenol, panthenyl ethyl ether, paraffin, PCA, peach (prunus persica) kernel oil, peanut (arachis hypogaea) oil, PEG-8 C12-18 ester, PEG-15 cocamine, PEG-150 distearate, PEG-60 glyceryl isostearate, PEG-5 glyceryl stearate, PEG-30 glyceryl stearate, PEG-7 hydrogenated castor oil, PEG-40 hydrogenated castor oil, PEG-60 hydrogenated castor oil, PEG-20 methyl glucose sesquistearate, PEG40 sorbitan peroleate, PEG-5 soy sterol, PEG-10 soy sterol, PEG-2 stearate, PEG-8 stearate, PEG-20 stearate, PEG-32 stearate, PEG40 stearate, PEG-50 stearate, PEG-100 stearate, PEG-150 stearate, pentadecalactone, peppermint (mentha piperita) oil, petrolatum, phospholipids, polyamino sugar condensate, polyglyceryl-3 diisostearate, polyquaternium-24, polysorbate 20, polysorbate 40, polysorbate 60, polysorbate 80, polysorbate 85, potassium myristate, potassium palmitate, potassium sorbate, potassium stearate, propylene glycol, propylene glycol dicaprylate/dicaprate, propylene glycol dioctanoate, propylene glycol dipelargonate, propylene glycol laurate, propylene glycol stearate, propylene glycol stearate SE, PVP, pyridoxine dipalmitate, quaternium-15, quaternium-18 hectorite, quaternium-22, retinol, retinyl palmitate, rice (oryza sativa) bran oil, RNA, rosemary (rosmarinus officinalis) oil, rose oil, safflower (carthamus tinctorius) oil, sage (salvia officinalis) oil, salicylic acid, sandalwood (santalum album) oil, serine, serum protein, sesame (sesamum indicum) oil, shea butter (butyrospermum parkii), silk powder, sodium chondroitin sulfate, sodium hyaluronate, sodium lactate, sodium palmitate, sodium PCA, sodium polyglutamate, sodium stearate, soluble collagen, sorbic acid, sorbitan laurate, sorbitan oleate, sorbitan palmitate, sorbitan sesquioleate, sorbitan stearate, sorbitol, soybean (glycine soja) oil, sphingolipids, squalane, squalene, stearamide MEA-stearate, stearic acid, stearoxy dimethicone, stearoxytrimethylsilane, stearyl alcohol, stearyl glycyrrhetinate, stearyl heptanoate, stearyl stearate, sunflower (helianthus annuus) seed oil, sweet almond (prunus amygdalus dulcis) oil, synthetic beeswax, tocopherol, tocopheryl acetate, tocopheryl linoleate, tribehenin, tridecyl neopentanoate, tridecyl stearate, triethanolamine, tristearin, urea, vegetable oil, water, waxes, wheat (triticum vulgare) germ oil, and ylang ylang (cananga odorata) oil.

In some embodiments, the compositions of the present disclosure may comprise one or more structuring agent. Structuring agents, in certain aspects, may assist in providing rheological characteristics to the composition to contribute to the composition's stability. In other aspects, structuring agents can also function as an emulsifier or surfactant. Non-limiting examples of structuring agents include stearic acid, palmitic acid, stearyl alcohol, cetyl alcohol, behenyl alcohol, stearic acid, palmitic acid, the polyethylene glycol ether of stearyl alcohol having an average of about 1 to about 21 ethylene oxide units, the polyethylene glycol ether of cetyl alcohol having an average of about 1 to about 5 ethylene oxide units, and mixtures thereof.

In some embodiments, the topical composition disclosed herein can comprise one or more amino acids. Exemplary amino acids include, but are not limited to, Asparagine, Tyrosine, Cysteine, Cystine, Serine, Threonine, Glutamine, Histidine, Glutamic Acid, Glutamate, Arginine, Lysine, Aspartic acid, Aspartate, Tryptophan, Isoleucine, Methionine, Proline, Phenylalanine, Glycine, Alanine, Valine, Leucine, aminobutyric acid, gamma aminobutyric acid, 2-aminobutyric acid, Dehydralanine, or any compound of the general formula H₂N—CRR′—CO₂H, or an acid, base, or salt thereof. In some embodiments, an amino acid is present as a potassium magnesium, lithium, manganese, or sodium salt. In some embodiments an amino acid is glutamic acid. In some embodiments, an amino acid is monosodium or disodium glutamate. In some embodiments, an amino acid is potassium glutamate. In some embodiments, an amino acid is dipotassium glutamate. In some embodiments an amino acid is aspartic acid. In some embodiments, an amino acid is monosodium or disodium aspartate. In some embodiments, an amino acid is potassium aspartate. In some embodiments, an amino acid is dipotassium aspartate.

In some embodiments, the topical composition disclosed herein can comprise at least 0.1% (w/w), 0.2% (w/w), 0.3% (w/w), 0.4% (w/w), 0.5% (w/w), 0.6% (w/w), 0.7% (w/w), 0.8% (w/w), 0.9% (w/w), 1.0% (w/w), 1.1% (w/w), 1.2% (w/w), 1.3% (w/w), 1.4% (w/w), 1.5% (w/w), 1.6% (w/w), 1.7% (w/w), 1.8% (w/w), 1.9% (w/w), 2.0% (w/w), 2.1% (w/w), 2.2% (w/w), 2.3% (w/w), 2.4% (w/w), 2.5% (w/w), 2.6% (w/w), 2.7% (w/w), 2.8% (w/w), 2.9% (w/w), 3.0% (w/w), 3.1% (w/w), 3.2% (w/w), 3.3% (w/w), 3.4% (w/w), 3.5% (w/w), 3.6% (w/w), 3.7% (w/w), 3.8% (w/w), 3.9% (w/w), or 4.0% (w/w) of an amino acid. In some embodiments, the topical composition disclosed herein can comprise 0.1% (w/w), 0.2% (w/w), 0.3% (w/w), 0.4% (w/w), 0.5% (w/w), 0.6% (w/w), 0.7% (w/w), 0.8% (w/w), 0.9% (w/w), 1.0% (w/w), 1.1% (w/w), 1.2% (w/w), 1.3% (w/w), 1.4% (w/w), 1.5% (w/w), 1.6% (w/w), 1.7% (w/w), 1.8% (w/w), 1.9% (w/w), 2.0% (w/w), 2.1% (w/w), 2.2% (w/w), 2.3% (w/w), 2.4% (w/w), 2.5% (w/w), 2.6% (w/w), 2.7% (w/w), 2.8% (w/w), 2.9% (w/w), 3.0% (w/w), 3.1% (w/w), 3.2% (w/w), 3.3% (w/w), 3.4% (w/w), 3.5% (w/w), 3.6% (w/w), 3.7% (w/w), 3.8% (w/w), 3.9% (w/w), 4% (w/w), 5% (w/w), 6% (w/w), 7% (w/w), 8% (w/w), 9% (w/w), 10% (w/w), 20% (w/w) or 30% (w/w) of an amino acid or a range defined by any two of the preceding values.

In some embodiments, the compositions disclosed herein may comprise an inert particle. An inert particle may have the same range of sizes, specific surface areas, pore sizes, pore densities, etc. as disclosed herein for an active particle. As contemplated herein, an inert particle does not comprise an active drug substance, though it may have an active drug substance incorporated into its structure, adhered to its surface, present within a pore, etc. Representative inert particles include but are not limited to metallic nanoparticles or microparticles such as Fe, Au, Cu, etc.; polymer nanoparticles or microparticles such as PLA, PLGA, PLA-PLGA, block copolymer, or acrylate nanoparticles or microparticles, silica, fumed silica, or amorphous silica nanoparticles or microparticles; proteinaceous or polysaccharide microparticles or nanoparticles such as dextran, collagen, chitosan, chitin, cellulose, or microcrystalline cellulose nanoparticles or microparticles; or other such particles as are known in the art. In some embodiments, an inert particle may be a silica particle. In some embodiments, an inert particle may be a fumed silica particle. In some embodiments, an inert particle may be either a hydrophilic or a hydrophobic fumed silica particle.

In some embodiments, the topical composition disclosed herein can comprise at least 0.1% (w/w), 0.2% (w/w), 0.3% (w/w), 0.4% (w/w), 0.5% (w/w), 0.6% (w/w), 0.7% (w/w), 0.8% (w/w), 0.9% (w/w), 1.0% (w/w), 1.1% (w/w), 1.2% (w/w), 1.3% (w/w), 1.4% (w/w), 1.5% (w/w), 1.6% (w/w), 1.7% (w/w), 1.8% (w/w), 1.9% (w/w), 2.0% (w/w), 2.1% (w/w), 2.2% (w/w), 2.3% (w/w), 2.4% (w/w), 2.5% (w/w), 2.6% (w/w), 2.7% (w/w), 2.8% (w/w), 2.9% (w/w), 3.0% (w/w), 3.1% (w/w), 3.2% (w/w), 3.3% (w/w), 3.4% (w/w), 3.5% (w/w), 3.6% (w/w), 3.7% (w/w), 3.8% (w/w), 3.9% (w/w), or 4.0% (w/w) of an inert particle. In some embodiments, the topical composition disclosed herein can comprise 0.1% (w/w), 0.2% (w/w), 0.3% (w/w), 0.4% (w/w), 0.5% (w/w), 0.6% (w/w), 0.7% (w/w), 0.8% (w/w), 0.9% (w/w), 1.0% (w/w), 1.1% (w/w), 1.2% (w/w), 1.3% (w/w), 1.4% (w/w), 1.5% (w/w), 1.6% (w/w), 1.7% (w/w), 1.8% (w/w), 1.9% (w/w), 2.0% (w/w), 2.1% (w/w), 2.2% (w/w), 2.3% (w/w), 2.4% (w/w), 2.5% (w/w), 2.6% (w/w), 2.7% (w/w), 2.8% (w/w), 2.9% (w/w), 3.0% (w/w), 3.1% (w/w), 3.2% (w/w), 3.3% (w/w), 3.4% (w/w), 3.5% (w/w), 3.6% (w/w), 3.7% (w/w), 3.8% (w/w), 3.9% (w/w), 4% (w/w), 5% (w/w), 6% (w/w), 7% (w/w), 8% (w/w), 9% (w/w), 10% (w/w), 20% (w/w) or 30% (w/w) of an inert particle or a range defined by any two of the preceding values.

In some embodiments, a composition as disclosed herein can comprise at least 0.1% (w/w), 0.2% (w/w), 0.3% (w/w), 0.4% (w/w), 0.5% (w/w), 0.6% (w/w), 0.7% (w/w), 0.8% (w/w), 0.9% (w/w), 1.0% (w/w), 1.1% (w/w), or 1.2% (w/w) of a bacterial cell, strain, culture, isolate, medium, extract, lyophile, or derivative thereof. In some embodiments, the topical composition disclosed herein can comprise 0.1% (w/w), 0.2% (w/w), 0.3% (w/w), 0.4% (w/w), 0.5% (w/w), 0.6% (w/w), 0.7% (w/w), 0.8% (w/w), 0.9% (w/w), 1.0% (w/w), 1.1% (w/w), 1.2% (w/w), 1.5% (w/w), 2% (w/w), 3% (w/w), 4% (w/w), 5% (w/w), 6% (w/w), 7% (w/w), 8% (w/w), 9% (w/w), 10% (w/w), 20% (w/w) or 30% (w/w) of a bacterial cell, strain, culture, isolate, medium, extract, lyophile, or derivative thereof, or a range defined by any two of the preceding values.

In some embodiments, the excipients can include an emulsifier. Suitable emulsifiers are disclosed in, for example, in McCutcheon's Detergents and Emulsifiers, North American Edition, pp. 317-324 (1986), and the ICI Handbook, pp. 1673-1686, which are incorporated herein by reference in their entirety. In some embodiments, the emulsifier can include glycerol monostearate. In some embodiments, the emulsifier can include polyoxyl stearate. In some embodiments, the emulsifier can include glycerol monostearate and polyoxyl stearate. In some embodiments, the emulsifier can include PEG-6 Stearate and Glycol stearate and PEG-32 stearate. In some embodiments, the emulsifier can include glycerol monostearate, PEG-6 Stearate, Glycol stearate and PEG-32 stearate.

In some embodiments, a composition as disclosed herein can comprise at least 0.1% (w/w), 0.2% (w/w), 0.3% (w/w), 0.4% (w/w), 0.5% (w/w), 0.6% (w/w), 0.7% (w/w), 0.8% (w/w), 0.9% (w/w), 1% (w/w), 2% (w/w), 3% (w/w), 4% (w/w), 5% (w/w), 6% (w/w), 7% (w/w), 8% (w/w), 9% (w/w), 10% (w/w), 11% (w/w), 12% (w/w), 13% (w/w), 14% (w/w), 15% (w/w), 16% (w/w), 17% (w/w), 18% (w/w), 19% (w/w), 20% (w/w), 30% (w/w), or 40% (w/w) of an emulsifier. In some embodiments, the topical composition disclosed herein can comprise 0.1% (w/w), 0.2% (w/w), 0.3% (w/w), 0.4% (w/w), 0.5% (w/w), 0.6% (w/w), 0.7% (w/w), 0.8% (w/w), 0.9% (w/w), 1% (w/w), 2% (w/w), 3% (w/w), 4% (w/w), 5% (w/w), 6% (w/w), 7% (w/w), 8% (w/w), 9% (w/w), 10% (w/w), 11% (w/w), 12% (w/w), 13% (w/w), 14% (w/w), 15% (w/w), 16% (w/w), 17% (w/w), 18% (w/w), 19% (w/w), 20% (w/w), 30% (w/w), or 40% (w/w) of an emulsifier or a range defined by any two of the preceding values. In some embodiments, the emulsifier can include one or more components, two or more components or three or more components.

In some embodiments, a composition as disclosed herein can comprise at least 0.1% (w/w), 0.2% (w/w), 0.3% (w/w), 0.4% (w/w), 0.5% (w/w), 0.6% (w/w), 0.7% (w/w), 0.8% (w/w), 0.9% (w/w), 1% (w/w), 2% (w/w), 3% (w/w), 4% (w/w), 5% (w/w), 6% (w/w), 7% (w/w), 8% (w/w), 9% (w/w), 10% (w/w), 11% (w/w), 12% (w/w), 13% (w/w), 14% (w/w), 15% (w/w), 16% (w/w), 17% (w/w), 18% (w/w), 19% (w/w), 20% (w/w), 30% (w/w), or 40% (w/w) of an emulsifier including glycerol monostearate, PEG-6 Stearate, Glycol stearate and PEG-32 stearate. In some embodiments, the topical composition disclosed herein can comprise 0.1% (w/w), 0.2% (w/w), 0.3% (w/w), 0.4% (w/w), 0.5% (w/w), 0.6% (w/w), 0.7% (w/w), 0.8% (w/w), 0.9% (w/w), 1% (w/w), 2% (w/w), 3% (w/w), 4% (w/w), 5% (w/w), 6% (w/w), 7% (w/w), 8% (w/w), 9% (w/w), 10% (w/w), 11% (w/w), 12% (w/w), 13% (w/w), 14% (w/w), 15% (w/w), 16% (w/w), 17% (w/w), 18% (w/w), 19% (w/w), 20% (w/w), 30% (w/w), or 40% (w/w) of an emulsifier including glycerol monostearate, PEG-6 Stearate, Glycol stearate and PEG-32 stearate or a range defined by any two of the preceding values. In some embodiments, the compositions described herein may comprise a mixture of glycerol monostearate, PEG-6 Stearate, Glycol stearate and PEG-32 stearate.

Further examples of emulsifiers contemplated herein may include, in some embodiments, esters of glycerin, esters of propylene glycol, fatty acid esters of polyethylene glycol, fatty acid esters of polypropylene glycol, esters of sorbitol, esters of sorbitan anhydrides, carboxylic acid copolymers, esters and ethers of glucose, ethoxylated ethers, ethoxylated alcohols, alkyl phosphates, polyoxyethylene fatty ether phosphates, fatty acid amides, acyl lactylates, soaps, TEA stearate, DEA oleth-3 phosphate, polyethylene glycol 20 sorbitan monolaurate (polysorbate 20), polyethylene glycol 5 soya sterol, steareth-2, steareth-20, steareth-21, ceteareth-20, PPG-2 methyl glucose ether distearate, ceteth-10, polysorbate 80, cetyl phosphate, potassium cetyl phosphate, diethanolamine cetyl phosphate, polysorbate 60, glyceryl stearate, PEG-100 stearate, and mixtures thereof.

In some embodiments, the compositions of the present disclosure may comprise one or more silicone containing compounds. Silicone containing compounds include any member of a family of polymeric products whose molecular backbone comprises silicon and carbon atoms with side groups attached to the silicon or carbon atoms. By varying the chain lengths, side groups, and crosslinking, silicones can be synthesized into a wide variety of materials. They can vary in consistency from liquid to gel to solids.

Silicone containing compounds that may be incorporated into some embodiments of the compositions of the present disclosure include those described herein, and/or those known to a person of ordinary skill in the art. Non-limiting examples include silicone oils (e.g., volatile and non-volatile oils), gels, and solids. In preferred aspects, the silicon containing compounds includes a silicone oils such as a polyorganosiloxane. Non-limiting examples of polyorganosiloxanes include dimethicone, cyclomethicone, polysilicone-11, phenyl trimethicone, trimethylsilylamodimethicone, stearoxytrimethylsilane, or mixtures of these and other organosiloxane materials in any given ratio in order to achieve the desired consistency and application characteristics depending upon the intended application (e.g., to a particular area such as the skin, hair, or eyes). A “volatile silicone oil” includes a silicone oil have a low heat of vaporization, i.e. normally less than about 50 cal per gram of silicone oil. Non-limiting examples of volatile silicone oils include: cyclomethicones such as Dow Corning 344 Fluid, Dow Corning 345 Fluid, Dow Corning 244 Fluid, and Dow Corning 245 Fluid, Volatile Silicon 7207 (Union Carbide Corp., Danbury, Conn.); low viscosity dimethicones, i.e. dimethicones having a viscosity of about 50 cst or less (e.g., dimethicones such as Dow Corning 200-0.5 cst Fluid). The Dow Corning Fluids are available from Dow Corning Corporation, Midland, Mich. Cyclomethicone and dimethicone are described in the Third Edition of the CTFA Cosmetic Ingredient Dictionary (incorporated by reference) as cyclic dimethyl polysiloxane compounds and a mixture of fully methylated linear siloxane polymers end-blocked with trimethylsiloxy units, respectively. Other non-limiting volatile silicone oils that can be used in the context of the present invention include those available from General Electric Co., Silicone Products Div., Waterford, N.Y. and SWS Silicones Div. of Stauffer Chemical Co., Adrian, Mich.

In some embodiments, the excipients can include preservatives. In some embodiments, the preservatives can be selected from the group consisting of benzyl alcohol, paraben, methyl paraben, propyl paraben, DMDM hydantoin, methylchloroisothiaoline, methylisothiazolinone, tocopherols, tocotrienols, imidazolidinyl urea phenoxyethanol, sodium benzoate and benzoic acid. In some embodiments, the preservatives can include phenoxyethanol, propyl paraben, and methyl paraben. In some embodiments, the preservatives can include benzalkonium chloride and/or poly(hexamethylenebiguanide) hydrochloride (PHMB). In some embodiments, the compositions described herein may comprise one or more tocopherols, including alpha tocopherol, beta tocopherol, delta tocopherol, gamma tocopherol, mixed tocopherols, tocotreinols, mixed tocotrienols, alpha tocotrienol, beta tocotrienol, delta tocotrienol, gamma tocotrienol, or mixtures thereof or any combination, derivative, or pro compound thereof. Exemplary tocopherol derivatives may include tocopheryl acetate, tocopheryl stearate, or other various tocopheryl compounds as are known in the art.

In some embodiments, a composition as disclosed herein can comprise at least 0.1% (w/w), 0.2% (w/w), 0.3% (w/w), 0.4% (w/w), 0.5% (w/w), 0.6% (w/w), 0.7% (w/w), 0.8% (w/w), 0.9% (w/w), 1.0% (w/w), 1.1% (w/w), or 1.2% (w/w) of a preservative or excipient capable of functioning as a preservative. In some embodiments, the topical composition disclosed herein can comprise 0.1% (w/w), 0.2% (w/w), 0.3% (w/w), 0.4% (w/w), 0.5% (w/w), 0.6% (w/w), 0.7% (w/w), 0.8% (w/w), 0.9% (w/w), 1.0% (w/w), 1.1% (w/w), 1.2% (w/w), 1.5% (w/w), 2% (w/w), 3% (w/w), 4% (w/w), 5% (w/w), 6% (w/w), 7% (w/w), 8% (w/w), 9% (w/w), 10% (w/w), 20% (w/w) or 30% (w/w) of a preservative or excipient capable of functioning as a preservative or a range defined by any two of the preceding values. In some embodiments, the preservative or excipient capable of functioning as a preservative can include one or more components, two or more components or three or more components. In some embodiments, a composition as disclosed herein may be free of, or substantially free of, preservatives, or of excipients capable of functioning as preservatives.

In some embodiments, a composition as disclosed herein can comprise at least 0.1% (w/w), 0.2% (w/w), 0.3% (w/w), 0.4% (w/w), 0.5% (w/w), 0.6% (w/w), 0.7% (w/w), 0.8% (w/w), 0.9% (w/w), 1.0% (w/w), 1.1% (w/w), or 1.2% (w/w) of a preservative including phenoxyethanol, propyl paraben, and methyl paraben. In some embodiments, the topical composition disclosed herein can comprise 0.1% (w/w), 0.2% (w/w), 0.3% (w/w), 0.4% (w/w), 0.5% (w/w), 0.6% (w/w), 0.7% (w/w), 0.8% (w/w), 0.9% (w/w), 1.0% (w/w), 1.1% (w/w), 1.2% (w/w), 1.5% (w/w), 2% (w/w), 3% (w/w), 4% (w/w), 5% (w/w), 6% (w/w), 7% (w/w), 8% (w/w), 9% (w/w), 10% (w/w), 20% (w/w) or 30% (w/w) of a preservative including phenoxyethanol, propyl paraben, and methyl paraben or a range defined by any two of the preceding values.

In some embodiments, a composition as disclosed herein may include colorants, deodorants, fragrances, perfumes, anti-foaming agents, lubricants, natural moisturizing agents, skin conditioning agents, skin protectants, skin benefit agents, solvents, solubilizing agents, suspending agents, wetting agents, humectants, propellants, dyes, pigments, and combinations thereof.

In some embodiments, a composition as disclosed herein may comprise an aqueous component. For example, the composition can be a cream, lotion, ointment, conditioning shampoo, moisturizing hand soap, etc. In some embodiments, the topical composition disclosed herein can comprise about 35% (w/w) to about 90% (w/w), about 40% (w/w) to about 85% (w/w), about 45% (w/w) to about 80% (w/w), about 50% (w/w) to about 75% (w/w), about 55% (w/w) to about 70% (w/w), about 60% (w/w) to about 65% (w/w), or about 62% (w/w) of water. In some embodiments, the topical composition disclosed herein can comprise at least 50% (w/w), 55% (w/w), 60% (w/w), 65% (w/w), 70% (w/w), 75% (w/w), 80% (w/w), or 85% (w/w) of water. In some embodiments, the topical composition disclosed herein can comprise up to 50% (w/w), 55% (w/w), 60% (w/w), 65% (w/w), 70% (w/w), 75% (w/w), 80% (w/w), or 85% (w/w) of water. In some embodiments, the topical composition disclosed herein can comprise 50% (w/w), 55% (w/w), 60% (w/w), 65% (w/w), 70% (w/w), 75% (w/w), 80% (w/w), or 85% (w/w) of water or a range defined by any two of the preceding values.

Some embodiments provide a composition including excipients that may function as skin penetration enhancers.

Examples of excipients that may function as skin penetration enhancers include alcohols, fatty acids, fatty acid esters, polyols, sulphoxides, glyceryl monooleate, lauryl lactate, Dodecyl-2-(N,N-dimethyl)-amino propionate (DDAIP), N-(4-bromobenzoyl)-S,S-dimethyliminosulfurane, NexACT enhancers, 2-nonyl-1,3-dioxolane (SEPA®), 1-dodecylazacycloheptan-2-one (Azone®), pyrrolidones, essential oil, terpenes, terpenoids, oxazolidinones, urea and the like.

Further excipients that may function as skin penetration enhancers are known in the art and include, but are not limited to, monoglycerides, polyglycosylated glycerides, glyceryl monoethyl ether, polysorbates, beta-cyclodextrin, cyclopentadecalactone, alkyl-2-(N,N-disubstituted amino)-alkanoate ester, 2-(n-nonyl)-1,3-dioxolane, isopropyl myristate, terpinol, menthol, cineol, monoolein, sodium oleate, oleyl oleate, laurylcapram, bisabolol, capsaicin, and capsicum. Other examples of excipients that may function as skin penetration enhancers and a description of their mechanism of action may be found in Goodman and Barry, “Percutaneous Absorption,” in Mechanisms-Methodology-Drug Delivery, 2nd Edition, Bronaugh and Maibach, eds., 1989, pp. 567-593, Marcel Dekker, Inc., N.Y., which is incorporated herein by reference in its entirety.

In some embodiments, an excipient that may function as a skin penetration enhancer can be selected from the group consisting of n-octanol, D-limonene, oleic acid, cineol, isopropyl myristate, monooleate, monoolein, sodium oleate, oleyl oleate, laurylcapram, sodium lauryl sulfate, bisabolol, lauric acid, myristic acid, isopropyl palmitate, diisopropyl adipate, dimethyl isosorbide, propylene glycol, butylene glycol, polyethylene glycol, dipropylene glycol, ethoxydiglycol, and pentylene glycol or combinations thereof. In a typical embodiment, the skin penetration enhancer can be selected from the group consisting of oleic acid, laurocapram, sodium lauryl sulphate, bisabolol, lauric acid, myristic acid, isopropyl myristate, isopropyl palmitate, diisopropyl adipate, dimethyl isosorbide, propylene glycol, butylene glycol, polyethylene glycol, dipropylene glycol, ethoxydiglycol, and pentylene glycol, or combinations thereof.

Examples of suitable fatty acids include, but are not limited to, valeric acid, heptanoic acid, pelagonic acid, caproic acid, capric acid, lauric acid, myristic acid, stearic acid, oleic acid, and caprylic acid; and branched fatty acids, such as isovaleric acid, neopentanoic acid, neoheptanoic acid, neononanoic acid, trimethyl hexanoic acid, neodecanoic acid, and isostearic acid.

Examples of suitable fatty acid esters include but are not limited to, isopropyl n-butyrate, isopropyl n-hexanoate, isopropyl n-decanoate, isopropyl myristate, isopropyl palmitate, and octyldodecyl myristate; alkyl fatty acid esters such as ethyl acetate, butyl acetate, methyl acetate, methylvalerate, methylpropionate, diethyl sebacate, and ethyl oleate; and diisopropyl adipate and dimethyl isosorbide.

In some embodiments, the compositions of the present disclosure may comprise one or more thickening agents. Thickening agents, including thickener or gelling agents, include substances that can increase the viscosity of a composition. Thickeners include those that can increase the viscosity of a composition without substantially modifying the efficacy of the active ingredient within the composition. Thickeners can also increase the stability of the compositions of the present invention. Non-limiting examples of additional thickening agents include carboxylic acid polymers, crosslinked polyacrylate polymers, polyacrylamide polymers, polysaccharides, and gums. Examples of carboxylic acid polymers include crosslinked compounds containing one or more monomers derived from acrylic acid, substituted acrylic acids, and salts and esters of these acrylic acids and the substituted acrylic acids, wherein the crosslinking agent contains two or more carbon-carbon double bonds and is derived from a polyhydric alcohol (see, e.g., U.S. Pat. Nos. 5,087,445; 4,509,949; 2,798,053; CTFA International Cosmetic Ingredient Dictionary, Fourth edition, 1991, pp. 12 and 80, each of which is incorporated by reference with respect to its disclosure of thickening agents and/or cross linked polymers). Examples of commercially available carboxylic acid polymers include carbomers, which are homopolymers of acrylic acid crosslinked with allyl ethers of sucrose or pentaerytritol (e.g., Carbopol™ 900 series from B.F. Goodrich). Non-limiting examples of crosslinked polyacrylate polymers include cationic and nonionic polymers. Examples are described in U.S. Pat. Nos. 5,100,660; 4,849,484; 4,835,206; 4,628,078; 4,599,379, each of which is incorporated by reference with respect to its disclosure of thickening agents and/or crosslinked polymers. Non-limiting examples of polyacrylamide polymers (including nonionic polyacrylamide polymers including substituted branched or unbranched polymers) include polyacrylamide, isoparaffin and laureth-7, multi-block copolymers of acrylamides and substituted acrylamides with acrylic acids and substituted acrylic acids. Non-limiting examples of polysaccharides include cellulose, carboxymethyl hydroxyethylcellulose, cellulose acetate propionate carboxylate, hydroxyethylcellulose, hydroxyethyl ethylcellulo se, hydroxypropylcellulo se, hydroxypropyl methylcellulose, methyl hydroxyethylcellulo se, microcrystalline cellulose, sodium cellulose sulfate, and mixtures thereof. Another example is an alkyl substituted cellulose where the hydroxy groups of the cellulose polymer is hydroxyalkylated (preferably hydroxy ethylated or hydroxypropylated) to form a hydroxyalkylated cellulose which is then further modified with a C10-C30 straight chain or branched chain alkyl group through an ether linkage. In some embodiments, these polymers are ethers of C10-C30 straight or branched chain alcohols with hydroxyalkylcelluloses. Other useful polysaccharides include scleroglucans comprising a linear chain of (1-3) linked glucose units with a (1-6) linked glucose every three unit. Non-limiting examples of gums that can be used with the present invention include acacia, agar, algin, alginic acid, ammonium alginate, amylopectin, calcium alginate, calcium carrageenan, carnitine, carrageenan, dextrin, gelatin, gellan gum, guar gum, guar hydroxypropyltrimonium chloride, hectorite, hyaluroinic acid, hydrated silica, hydroxypropyl chitosan, hydroxypropyl guar, karaya gum, kelp, locust bean gum, natto gum, potassium alginate, potassium carrageenan, propylene glycol alginate, sclerotium gum, sodium carboyxmethyl dextran, sodium carrageenan, tragacanth gum, xanthan gum, and mixtures thereof.

In some embodiments, optimal delivery of an active drug substance to the site of a disease or disorder implicating dysbiosis of the skin can be achieved by formulating the compositions disclosed herein into a nanoparticle or nanoemulsion. Such particles and emulsions are known in the art and include but are not limited to, polylactic acid particles, polylactic/glycolic acid nanoparticles, polystyrene nanoparticles, silicon dioxide nanoparticles, metallic nanoparticles, water-in-oil emulsions, oil-in-water emulsions, polymer nanoparticles and emulsions, and block copolymer nanoparticles and emulsions.

In some embodiments, a composition as disclosed herein can comprise at least 5% (w/w), 6% (w/w), 7% (w/w), 8% (w/w), 9% (w/w), 10% (w/w), 11% (w/w), or 12% (w/w) of one or more excipients that may function as skin penetration enhancer. In some embodiments, a composition as disclosed herein can comprise 5% (w/w), 6% (w/w), 7% (w/w), 8% (w/w), 9% (w/w), 10% (w/w), 11% (w/w), 12% (w/w), 13% (w/w), 14% (w/w), 15% (w/w), 16% (w/w), 17% (w/w), 18% (w/w), 19% (w/w), 20% (w/w), 21% (w/w), 22% (w/w), 23% (w/w), 24% (w/w), 25% (w/w), 26% (w/w), 27% (w/w), 28% (w/w), 29% (w/w), or 30% (w/w)) of one or more excipients that may function as skin penetration enhancers or a range defined by any two of the preceding values. In a typical embodiment, the skin penetration enhancer can be ethoxydiglycol.

In some embodiments as contemplated herein, one or more excipients disclosed herein may be present without regard to their function in enhancing or modulating skin penetration. In some embodiments, compositions as disclosed herein may incorporate one or more of the compounds disclosed herein without providing significant penetration of, into, or through the skin. In some embodiments, skin penetration is limited to the stratum corneum. In some embodiments, skin penetration is limited to hair follicles and infection sites. In some embodiments, skin penetration is limited to intermediate dermal layers. In some embodiments, skin penetration is limited to the epidermis. In some embodiments, the compositions as disclosed herein allow penetration of the active drug substance throughout the dermis.

In some embodiments, a composition as disclosed herein can comprise at least 0.01% (w/w), 01% (w/w), 1% (w/w), 2% (w/w), 3% (w/w), 4% (w/w), 5% (w/w), 6% (w/w), 7% (w/w), 8% (w/w), 9% (w/w), 10% (w/w), 11% (w/w), or 12% (w/w) of one or more excipients that may function as an antioxidant. In some embodiments, a composition as disclosed herein can comprise 0.01% (w/w), 01% (w/w), 1% (w/w), 2% (w/w), 3% (w/w), 4% (w/w), 5% (w/w), 6% (w/w), 7% (w/w), 8% (w/w), 9% (w/w), 10% (w/w), 11% (w/w), 12% (w/w), 13% (w/w), 14% (w/w), 15% (w/w), 16% (w/w), 17% (w/w), 18% (w/w), 19% (w/w), 20% (w/w), 21% (w/w), 22% (w/w), 23% (w/w), 24% (w/w), 25% (w/w), 26% (w/w), 27% (w/w), 28% (w/w), 29% (w/w), or 30% (w/w)) of one or more excipients that may function as antioxidants or a range defined by any two of the preceding values. In some embodiment, an antioxidant may comprise one or more of selenium, vitamin A, vitamin E, vitamin C, retinyl palmitate, ascorbic acid, alpha-tocopherol, beta tocopherol, delta tocopherol, gamma tocopherol, tocotrienol, mixed tocopherols, mixed tocotrienols, other tocopherols,and/or tocotrienols, ethylenediaminetetraacetic acid (EDTA), ethylene glycol bis(2-aminoethyl)tetraacetic acid (EGTA), sodium metabisulfite, sodium bisulfite, citric acid, tartaric acid, or any combination thereof, or any esters or derivatives thereof, or any compound known in the art to bind, degrade, or otherwise shelter the active component of the formulation from, dioxygen, ozone, superoxide, exile radicals, reactive oxygen species, or other oxidative components as may be present in the environment and to which the compositions disclosed may be susceptible. In some embodiments, an antioxidant may comprise one or more of acetyl cysteine, ascorbic acid polypeptide, ascorbyl dipalmitate, ascorbyl methylsilanol pectinate, ascorbyl palmitate, ascorbyl stearate, BHA, BHT (butylated hydroxytoluene), t-butyl hydroquinone, cysteine, cysteine HCl, diamylhydroquinone, di-t-butylhydroquinone, dicetyl thiodipropionate, dioleyl tocopheryl methylsilanol, disodium ascorbyl sulfate, distearyl thiodipropionate, ditridecyl thiodipropionate, dodecyl gallate, erythorbic acid, esters of ascorbic acid, ethyl ferulate, ferulic acid, gallic acid esters, hydroquinone, isooctyl thioglycolate, kojic acid, magnesium ascorbate, magnesium ascorbyl phosphate, methylsilanol ascorbate, natural botanical anti-oxidants such as green tea or grape seed extracts, nordihydroguaiaretic acid, octyl gallate, phenylthioglycolic acid, potassium ascorbyl tocopheryl phosphate, potassium sulfite, propyl gallate, quinones, rosmarinic acid, sodium ascorbate, sodium bisulfite, sodium erythorbate, sodium metabisulfite, sodium sulfite, superoxide dismutase, sodium thioglycolate, sorbityl furfural, thiodiglycol, thiodiglycolamide, thiodiglycolic acid, thioglycolic acid, thiolactic acid, thiosalicylic acid, tocophereth-5, tocophereth-10, tocophereth-12, to cophereth-18, to cophereth-50, tocopherol, to cophersolan, tocopheryl acetate, tocopheryl linoleate, tocopheryl nicotinate, tocopheryl succinate, aor tris(nonylphenyl)phosphite or any combination thereof, or any derivative thereof.

In some embodiments, a composition may include additional components added to enhance the odor, texture or color of the composition. For example, fragrances may be added to enhance odor. For example, emulsifiers or inert spheres may be added to enhance texture. For example, colorants may be added to enhance color.

In some embodiments, the compositions of the present disclosure may comprise one or more essential oils. Essential oils include oils derived from herbs, flowers, trees, and other plants. Such oils are typically present as tiny droplets between the plant's cells, and can be extracted by several method known to those of skill in the art (e.g., steam distilled, enfleurage (i.e., extraction by using fat), maceration, solvent extraction, or mechanical pressing). When these types of oils are exposed to air they tend to evaporate (i.e., a volatile oil). As a result, many essential oils are colorless, but with age they can oxidize and become darker. Essential oils are insoluble in water and are soluble in alcohol, ether, fixed oils (vegetal), and other organic solvents. Typical physical characteristics found in essential oils include boiling points that vary from about 160° to 240° C. and densities ranging from about 0.759 to about 1.096.

Essential oils typically are named by the plant from which the oil is found. For example, rose oil or peppermint oil are derived from rose or peppermint plants, respectively. Non-limiting examples of essential oils contemplated as components of the compositions disclosed herein may include sesame oil, macadamia nut oil, tea tree oil, evening primrose oil, Spanish sageoil, Spanish rosemary oil, coriander oil, thyme oil, pimento berries oil, rose oil, anise oil, balsam oil, bergamot oil, rosewood oil, cedar oil, chamomile oil, cucumber oil, sage oil, clary sage oil, clove oil, cypress oil, eucalyptus oil, fennel oil, sea fennel oil, frankincense oil, geranium oil, ginger oil, grapefruit oil, jasmine oil, juniper oil, lavender oil, lemon oil, lemongrass oil, lime oil, mandarin oil, marjoram oil, myrrh oil, neroli oil, orange oil, patchouli oil, pepper oil, black pepper oil, petitgrain oil, pine oil, rose otto oil, rosemary oil, sandalwood oil, spearmint oil, spikenard oil, vetiver oil, wintergreen oil, or ylang ylang, or other such oils as are known to one of skill in the art, or any combination thereof.

The CTFA International Cosmetic Ingredient Dictionary and Handbook (2016), 16th Edition, which is hereby incorporated by reference in its entirety, describes a wide variety of non-limiting cosmetic ingredients that can be used in the context of the present invention. Examples of these ingredient classes include: fragrances (artificial and natural), dyes and color ingredients (e.g., Blue 1, Blue 1 Lake, Red 40, titanium dioxide, D&C blue no. 4, D&C green no. 5, D&C orange no. 4, D&C red no. 17, D&C red no. 33, D&C violet no. 2, D&C yellow no. 10, and D&C yellow no. 11), adsorbents, emulsifiers, stabilizers, lubricants, solvents, moisturizers (including, e.g., emollients, humectants, film formers, occlusive agents, and agents that affect the natural moisturization mechanisms of the skin), water-repellants, UV absorbers (physical and chemical absorbers such as paraminobenzoic acid (“PABA”) and corresponding PABA derivatives, titanium dioxide, zinc oxide, etc.), cinnamic acid and derivatives thereof, including p-hydroxycinnamic acid, essential oils, vitamins (e.g., A, B, C, D, E, and K), trace metals (e.g., zinc, calcium and selenium), anti-irritants (e.g., steroids and non-steroidal anti-inflammatories), botanical extracts (e.g., aloe vera, chamomile, cucumber extract, ginkgo biloba, ginseng, and rosemary), anti-microbial agents, antioxidants (e.g., BHT and tocopherol), chelating agents (e.g., disodium EDTA and tetrasodium EDTA), preservatives (e.g., methylparaben and propylparaben), pH adjusters (e.g., sodium hydroxide and citric acid), absorbents (e.g., aluminum starch octenylsuccinate, kaolin, corn starch, oat starch, cyclodextrin, talc, and zeolite), skin bleaching and lightening agents (e.g., hydroquinone and niacinamide lactate), humectants (e.g., glycerin, propylene glycol, butylene glycol, pentylene glycol, sorbitol, urea, and manitol), exfoliants (e.g., alpha-hydroxyacids, and beta-hydroxyacids such as lactic acid, glycolic acid, and salicylic acid; and salts thereof) waterproofing agents (e.g., magnesium/aluminum hydroxide stearate), skin conditioning agents (e.g., aloe extracts, allantoin, bisabolol, ceramides, dimethicone, hyaluronic acid, and dipotassium glycyrrhizate), thickening agents (e.g., substances which that can increase the viscosity of a composition such as carboxylic acid polymers, crosslinked polyacrylate polymers, polyacrylamide polymers, polysaccharides, and gums), and silicone containing compounds (e.g., silicone oils and polyorgano siloxanes).

In some embodiments, the compositions of the present disclosure may comprise one or more UV absorption agents. UV absorption agents that can be used in combination with the compositions of the present invention include chemical and physical sunblocks. Non-limiting examples of chemical sunblocks that can be used include para-aminobenzoic acid (PABA), PABA esters (glyceryl PABA, amyldimethyl PABA and octyldimethyl PABA), butyl PABA, ethyl PABA, ethyl dihydroxypropyl PABA, benzophenones (oxybenzone, sulisobenzone, benzophenone, and benzophenone-1 through 12), cinnamates (octyl methoxycinnamate, isoamyl p-methoxycinnamate, octylmethoxy cinnamate, cinoxate, diisopropyl methyl cinnamate, DEA-methoxycinnamate, ethyl diisopropylcinnamate, glyceryl octanoate dimethoxycinnamate and ethyl methoxycinnamate), cinnamate esters, salicylates (homomethyl salicylate, benzyl salicylate, glycol salicylate, isopropylbenzyl salicylate, etc.), anthranilates, ethyl urocanate, homosalate, octisalate, dibenzoylmethane derivatives (e.g., avobenzone), octocrylene, octyl triazone, digalloy trioleate, glyceryl aminobenzoate, lawsone with dihydroxyacetone, ethylhexyl triazone, dioctyl butamido triazone, benzylidene malonate polysiloxane, terephthalylidene dicamphor sulfonic acid, disodium phenyl dibenzimidazole tetrasulfonate, diethylamino hydroxybenzoyl hexyl benzoate, bis diethylamino hydroxybenzoyl benzoate, bis benzoxazoylphenyl ethylhexylimino triazine, drometrizole trisiloxane, methylene bis-benzotriazolyl tetramethylbutylphenol, and bis-ethylhexyloxyphenol methoxyphenyltriazine, 4-methylbenzylidenecamphor, and isopentyl 4-methoxycinnamate. Non-limiting examples of physical sunblocks include, kaolin, talc, petrolatum and metal oxides (e.g., titanium dioxide and zinc oxide). Compositions of the present invention can have UVA and UVB absorption properties. The compositions can have an sun protection factor (SPF) of 2, 3, 4, 56, 7, 8, 9, 10, 11, 12, 13, 14, 15, 20, 25, 30, 35, 40, 45, 50, 55, 60, 70, 80, 90 or more, or any integer or derivative therein.

In some embodiments, the compositions disclosed herein may be incorporated into products. Non-limiting examples of products include cosmetic products, food-based products (e.g., fortified water, energy drinks, nutritional drinks, vitamins, supplements, solid foods), pharmaceutical products, etc. In some embodiments, for example, cosmetic products include sunscreen products, sunless skin tanning products, hair products (e.g., shampoos, conditioners, colorants, dyes, bleaches, straighteners, and permanent wave products), fingernail products, moisturizing creams, skin creams and lotions, softeners, day lotions, gels, ointments, foundations, night creams, lipsticks and lip balms, cleansers, toners, masks, deodorants, antiperspirants, exfoliating compositions, shaving-related products (e.g., creams, “bracers” and aftershaves), pre-moistened wipes and washcloths, tanning lotions, bath products such as oils, foot care products such as powders and sprays, skin colorant and make-up products such as foundations, blushes, rouges eye shadows and lines, lip colors and mascaras, baby products (e.g., baby lotions, oils, shampoos, powders and wet wipes), and skin or facial peel products. Additionally, the cosmetic products can be formulated as leave-on or rinse-off products. Further products can include hair re-growth compositions, serums, sprays, and the like.

In some embodiments, the topical composition may be applied to a body portion, such as a hand, foot, knee, elbow, and the like to treat pain and/or inflammation of the body portion. The composition may be applied by any suitable means, such as rubbing, spraying, rolling, wiping, and the like, and massaged into the body portion to be treated.

In some embodiments, the compounds as disclosed and described herein and/or topical compositions thereof can be used in combination therapy with at least one other agent. In some embodiments, a compound as disclosed and described herein and/or topical composition thereof is administered concurrently with the administration of another agent, which may be part of the same topical composition as the compound of the present invention or a different composition. In some embodiments, a topical composition of the present invention is administered prior or subsequent to administration of another agent.

In some embodiments, the compositions of the present disclosure may comprise one or more pharmaceutical ingredients. In addition to those disclosures made elsewhere herein, non-limiting examples of pharmaceutical ingredients include anti-acne agents, agents used to treat rosacea, analgesics, anesthetics, anorectals, antihistamines, anti-inflammatory agents including non-steroidal anti-inflammatory drugs, antibiotics, antifungals, antivirals, antimicrobials, anti-cancer actives, scabicides, pediculicides, antineoplastics, antiperspirants, antipruritics, antipsoriatic agents, antiseborrheic agents, biologically active proteins and peptides, burn treatment agents, cauterizing agents, depigmenting agents, depilatories, diaper rash treatment agents, enzymes, hair growth stimulants, hair growth retardants including DFMO and its salts and analogs, hemostatics, kerotolytics, protease inhibitors, intercalating agents, nucleotide or nucleoside mimetics, canker sore treatment agents, cold sore treatment agents, dental and periodontal treatment agents, photosensitizing actives, skin protectant/barrier agents, steroids including hormones and corticosteroids, sunburn treatment agents, sunscreens, transdermal actives, nasal actives, vaginal actives, wart treatment agents, wound treatment agents, wound healing agents, etc. In some embodiments, the compositions disclosed herein may comprise any one or more of the compounds disclosed in U.S. Patent Application Publications No. 2018/0221331, 2018/0289751 and 2017/031221, each of which is hereby expressly incorporated by reference in its entirety and especially for their respective disclosures of pharmaceutically and/or biologically active compounds, as well as pharmaceutically acceptable and/or biocompatible excipients suitable for inclusion in the compositions of the present disclosure.

In some embodiments, the compositions of the present disclosure may be present in a container and/or a kit. A kit may include a container. Exemplary containers include a bottle, a metal tube, a laminate tube, a plastic tube, a dispenser, a pressurized container, a barrier container, a package, a compartment, a lipstick container, a compact container, cosmetic pans that can hold cosmetic compositions, or other types of containers such as injection or blow-molded plastic containers into which the dispersions or compositions or desired bottles, dispensers, or packages are retained. The kit and/or container may include indicia on its surface. The indicia, for example, can be a word, a phrase, an abbreviation, a picture, or a symbol. in some embodiments, a container according to the present disclosure may dispense a pre-determined amount of a composition. In some embodiments, such as, for example, a metal, laminate, or plastic tube, packet, or pouch, a container may be squeezed to dispense a desired amount of the composition. As contemplated herein, a composition may be dispensed as a spray, foam, an aerosol, a liquid, a fluid, or a semi-solid. A container may have spray, pump, or squeeze mechanisms. A kit may also include instructions for using the kit and/or compositions. Instructions can include an explanation of how to apply, use, and maintain the compositions.

In some embodiments, the compositions as disclosed herein are provided in an anaerobic form. In some embodiments, the compositions as disclosed herein are provided in a packaging or enclosure which limits the availability or presence of oxygen. In some embodiments, the compositions as disclosed herein comprise an oxygen scavenger. In some embodiments, said oxygen scavenger may be dissolved within the composition. In some embodiments, said oxygen scavenger may be a solid item or mass separate from the fluid mass of the composition. In some embodiments, said oxygen scavenger may be a device or material that is inserted within the packaging or enclosure. In some embodiments, the composition may be packaged in an environment lacking oxygen, such as under a nitrogen, argon, helium, xenon, or krypton overlay. In some embodiments, the composition may be packaged under a carbon dioxide, CF₃, CF₄, methane, ethane, propane, isopropane, butane, isobutane, pentane, or isopentane overlay.

In some embodiments, the compositions as disclosed herein are provided in a moisture-free or substantially moisture-free form. In some embodiments, the compositions as disclosed herein are provided in a packaging or enclosure which limits the availability or presence of water or moisture. In some embodiments, the compositions as disclosed herein comprise a water/moisture scavenger or drying agent. In some embodiments, said water/moisture scavenger or drying agent may be dissolved within the composition. In some embodiments, said water/moisture scavenger or drying agent may be a solid item or mass separate from the fluid mass of the composition. In some embodiments, said water/moisture scavenger or drying agent may be a device or material that is inserted within the packaging or enclosure. In some embodiments, the composition may be packaged in an environment lacking water or moisture, such as under a dry or substantially dry air, nitrogen, argon, helium, xenon, or krypton overlay. In some embodiments, the composition may be packaged under a carbon dioxide, CF₃, CF₄, methane, ethane, propane, isopropane, butane, isobutane, pentane, or isopentane overlay. In some embodiments, said water/moisture scavenger or drying agent may comprise an adsorbent, an anhydrous ceramic medium, a bentonite, charcoal, a polymer such as a polyurethane or polyurea, a zeolite, an oxazolidine, an active chemical scavenger, a silane (such as vinyltrimethoxysilane), a cellulose, a fiber, a molecular sieve composition, or other such compositions or elements as are known in the art for the reduction, removal, or prevention of moisture in pharmaceutical, cosmetic, chemical, or biological compositions.

According to the methods disclosed herein, a reduction in inflammation due to dysbiosis, including inflammation due to atopic dermatitis, may be achieved by modulating the dosing schedule such that subjects experience periodic partial or full reductions in dosing for fixed amounts of time, followed by a resumption of dosing. In some embodiments, one or more dosages are administered daily for between one and thirty days, followed by a dosing holiday lasting for between one and thirty days. In some embodiments, during the dosing holiday, no dose is administered. In some further embodiments, the composition of the present disclosure is allowed to clear completely from the subject's body prior to administration of the next dose. In some embodiments, during the dosing holiday, a dose less than the usual daily dose is administered. In some further embodiments, an amount of the administered composition less than the therapeutically effective amount is allowed to remain within the subject during the dosing holiday. In some further embodiments, an amount of the administered composition sufficient to maintain therapeutic levels in the affected tissues is allowed to remain within the subject.

According to the present disclosure, the dosing schedule can be varied so as to attain the desired therapeutic effect. In each of the embodiments as disclosed herein, variations in dosing schedule can be repeated throughout the duration of the therapeutic protocol being administered. In each of the embodiments as disclosed herein, the first dosage can be higher, lower, or the same as the dosages following the first dosage. In each of the embodiments disclosed herein, a loading dose may precede the disclosed dosing regimen, and a dosing holiday may or may not follow the administration of the loading dose.

In some embodiments the methods of the present disclosure comprise administration of the one or more compositions provided herein daily or less frequently than daily, such as every second day, every third day, every fourth day, every fifth day, every sixth day, or every seventh day or for a time period that is within a range defined by any two of the aforementioned times.

The methods described herein are further illustrated by the following non-limiting examples.

### EXAMPLES

Some aspects of the embodiments discussed above are disclosed in further detail in the following examples, which are not in any way intended to limit the scope of the present disclosure.

### Example 1

### Preparation of a Drug Substance

Multiple 10 L fermenters were inoculated with S. hominis strain A9. Cell pellets were collected by centrifugation, washed with 1× sterile saline solution, and resuspended in a solution of 4% (w/w) monosodium glutamate, 2% dextran 500, and 2% sorbitol. The resuspension was lyophilized and dry milled using sequential mesh screening through 20 mesh followed by 60 mesh screens. Milled, screened, lyophilized drug product was stored at −20° C.

### Example 2

### Evaluation of Fermentation Methods and Growth Media

Animal-free growth mediums A) AF-Tryptic Soy Broth, B) AF-Terrific Broth, and C) AF-Luria Broth were compared for Sh-A9 RCB growth evaluation in shaker flask cultures at 37° C. and 200 rpm. Dextrose and AF-peptone feed supplementation during exponential cell growth phase was also evaluated in shaker flask cultures using the RCB vials. Result: AF-TSB out performed other AF-peptone based mediums as evaluated by determining CFU/mL over time and diameter of zones of inhibition on S. aureus strain Sa 113 UCSD lawns. Dextrose and peptone feeding mid-culture did not result in a significant increase in zone of inhibition area on Sa 113 UCSD lawns. All cell banks grew CFUs which inhibited Staph aureus 113 UCSD in zone of inhibition assay lawns, had the same genome sequence and contained the hogocidin lantibiotic operon.

Sh-A9 growth in different concentrations of AF-TSB (1× to 5×) were also evaluated. Maximum cell biomass produced in each TSB concentration was determined by both measuring CFU/mL and by mg wet weight cells/mL culture upon centrifugation of cell biomass. In addition, anti-SA activity (which may be attributable to hogocidin AMP/lantibiotic or other antimicrobial activity) secreted into growth medium was assayed in 0.2 μ filtered Sh-A9 conditioned medium by measuring zones of Staph aureus growth inhibition on Sa 113 UCSD lawns.

SH-A9 conditioned medium harvested after overnight growth (>16 hour) in either 1×, 2×, 3× or 4× AF-TSB were filter sterilized and aliquoted into tubes. A 10% volume of a 10-fold dilution series of stationary phase USA/300 MRSA cells were added to the tubes containing the conditioned media aliquots. Tubes containing USA/300 cells in the 4 different Sh-A9 conditioned media or in fresh TSB were incubated 16 hours at 30° C. followed by reading OD600 as a measure of the ability of Sh-A9 conditioned media to inhibit the growth of USA/300 MRSA.

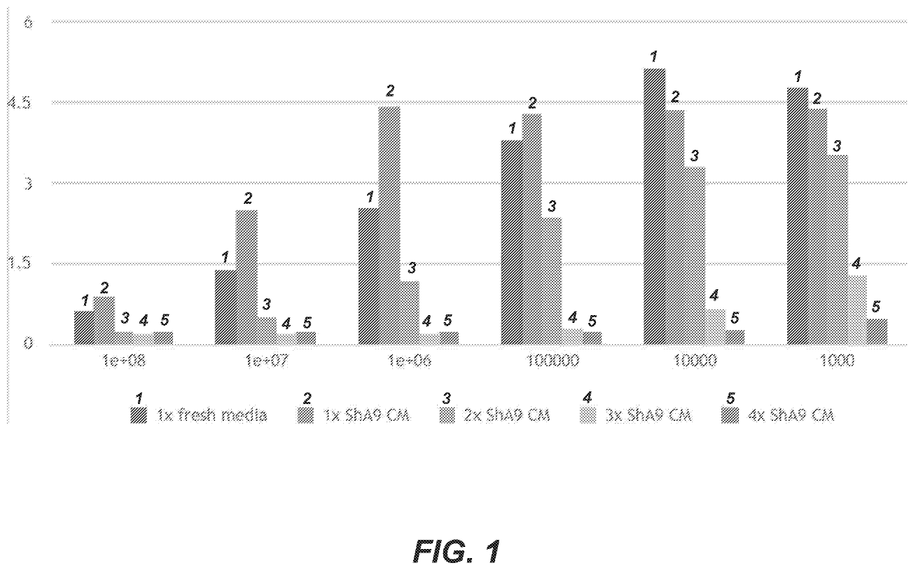

Sh-A9 growth in AF-TSB of increasing concentration secrete relatively greater antimicrobial activity into the conditioned medium compared to the same seed of Sh-A9 grown in standard 1× AF-TSB. This activity is measurable by assaying Staph aureus growth inhibition on Sa 113 UCSD lawns or using a turbidity based growth assay using USA/300 MRSA UCSD as the test strain (FIG. 1).

### Example 3

### Preparation of a Drug Substance

Multiple 5-liter sparged aerobic bioreactor development processes were run using AF-TSB (Corning) growth medium. Initial process development used air sparging with increasing agitation rates, with a final process using sparged oxygen and air at high volumes and flow rates with low radial agitation at 37° C. with anti-foam control. Cells were harvested after 8 hours at 37° C. following a 1 to 50 inoculation into the bioreactor from a seed flask culture.

OD₆₀₀ turbidity in the bioreactor after 8 hours sparged oxygen growth ranged from <8 to >10 with CFU/mL ranging <1×10⁹ CFU/mL to >6×10⁹ CFU/mL. Harvest was performed prior to bioreactor culture reaching stationary growth phase which was demonstrated by decreasing CFU/mL over time in earlier-run processes.

In a second study, four 10-liter and one 5-liter sparged oxygen/air final process parameter batches were run. Inoculation of the bioreactor at 1 to 50 with a seed shaker flask consistently reached harvest OD6₀₀ specification of >8 and <10 in ˜6 hours in all 5 processes performed.

Washed cell biomass from each run was split and freeze dried in either of 2 cryopreservation buffers described below (Example 7) and resulted in milled/sieved bacterial powders (drug substances) for product formulation development and stability assessment. Representative results are shown in table 1, below.

TABLE 1

| Sample | | | | |
|---|---|---|---|---|
| Type | Lot # | CFU | Total Cell Count | Moisture |
| Lyophilized | CO-25-07 | 1.14E+11 CFU/g | 3.65E+11 cells/g | 5.09% |
| Material | CO-25-08 | 1.69E+11 CFU/g | 3.13E+11 cells/g | 5.44% |
| | CO-25-10 | 1.60E+11 CFU/g | 2.80E+11 cells/g | 6.63% |
| Cell Bank | WCB | 3.8E+8 CFU/ml | 1.55E+9 cells/ml | N/A |
| | MCB | 4.15E+8 CFU/ml | 1.47E+9 cells/ml | N/A |

### Example 4

### Preparation of a Drug Product

Cell harvest can be performed by either centrifugation or using Hollow Fiber Tangential Filtration Membranes depending on manufacturing vessel size and equipment. Collected Sh-A9 cells are washed with sterile saline (0.9% USP) prior to mixing the resulting cell paste with 3 parts 4× Lyophilizing Sugar Buffer (w/w) to prepare a 1× Sh-A9 cell-sugar buffer slurry.

Lyophilization of 1× Sh-A9 cells in sugar buffer slurry is performed in pre-weighed Lyo-Guard Trays (Gore Inc.). Batches of 1× Sh-A9 cell-sugar buffer slurries poured into Lyo-Guard Trays are placed immediately into the lyophilizer and the cycle started by decreasing the temperature to −40° C. in 60 minutes (1° C./minute). Hold time at -40° C. is 6 to 18 hours depending on volume. Drying cycle steps are based on pressure differentials rather than based on time. The amount of drying time is not significantly different despite use of time versus pressure differentials.

Lyophilized Sh-A9 in Lyo-Guard Trays are stored at −20° C. with desiccant packs in foil bags. Lyophilized Sh-A9 cakes are fragmented in the Lyo-Guard Trays and then milled through sterile 20 mesh followed by 60 or 100 mesh stainless steel sieve screens to produce bulk Sh-A9 drug substance (DS). Bulk Sh-A9 DS Powder is weighed into sterile plastic pouches and stored at −20° C. with desiccant under N₂ gas or vacuum. Sh-A9 DS powder is stable at −20° C. for at least 12 months.

### Example 5

### Development of an Anhydrous Lotion Formulation

A drug substance was prepared as in Example 1. An anhydrous lotion composition was prepared by mixing 64% (w/w) Soy Oil, 7% (w/w) Stearyl Alcohol, 7% (w/w) Cetostearyl Alcohol, and 1% (w/w) Tocopherol (Mixture 1) under N₂ overlay at >45° C.; and 20% (w/w) Soy Oil, 0.5% (w/w) Colloidal Silicon Dioxide, and 0.5% (w/w) Drug Substance (Mixture 2) under N₂ overlay at 25° C. Mixture 1 was cooled to 25° C. and both mixtures were combined under N₂ overlay and stored under N₂ overlay. In this example, w/w refers to weight of a component divided by the weight of the final combined composition.

Similarly, an anhydrous lotion placebo composition was prepared by mixing 64% (w/w) Soy Oil, 7% (w/w) Stearyl Alcohol, 7% (w/w) Cetostearyl Alcohol, and 1% (w/w) Tocopherol (Mixture 1) under N₂ overlay at >45° C.; and 20% (w/w) Soy Oil, 0.5% (w/w) Colloidal Silicon Dioxide, and by mixing either with or without 0.5% (w/w) agar powder (Mixture 2) under N₂ overlay at 25° C. until a homogenous suspension was formed. Mixture 1 was cooled to 25° C. and both mixtures were combined under N₂ overlay and stored under N₂ overlay. In this example, w/w refers to weight of a component divided by the weight of the final combined composition.

### Example 6

### Development of an Anhydrous Oil Formulation

A drug substance was prepared as in Example 1. An anhydrous oil composition was prepared by mixing 98% (w/w) soy oil, 1% (w/w) Tocopherol, 0.5% (w/w) Colloidal SiO₂, and 0.5% (w/w) Drug Substance. The composition was capped under N₂ overlay.

Similarly, an anhydrous oil placebo composition was prepared by mixing 98% (w/w) Soy Oil , 1% (w/w) Tocopherol, 0.5% (w/w) Colloidal SiO₂, and 0.5% (w/w) agar powder. The composition was capped under N₂ overlay. Once Mixture 1 was cooled to below 25° C., Mixture 2 was poured into Mixture 1 and components were stirred under N₂ overlay until a uniform gel formed incorporating the extra soy oil, colloidal silica, and placebo drug substance mixture evenly into an anhydrous gel matrix.

An alternative placebo oil composition was produced by combining 0.5% (w/w) MSG, Sorbitol, and Dextran-500 in soy oil.

### Example 7

### Cryoprotectant Identification

Bacterial cell pellets were resuspended in four different media to assess freezing, survival, and stability of the lyophile, as well as recovery of bacterial growth after lyophilization and storage.

Freeze Media #1 1×: 10% sucrose, 5% AF-Soytone (Corning)

Freeze Media #2 1×: 10% sucrose, 3% AF-Soytone (Corning), 2.5% (mono)sodium glutamate, 4% ascorbic acid

Freeze Media #3 1×: 4% (mono)sodium glutamate, 2% Dextran 500, 2% sorbitol

Freeze Media #3 1×: 1% (mono)sodium glutamate, 2.5% Dextran 64-76K, 0.8% Sucrose, 0.42% Histidine, 5% Mannitol

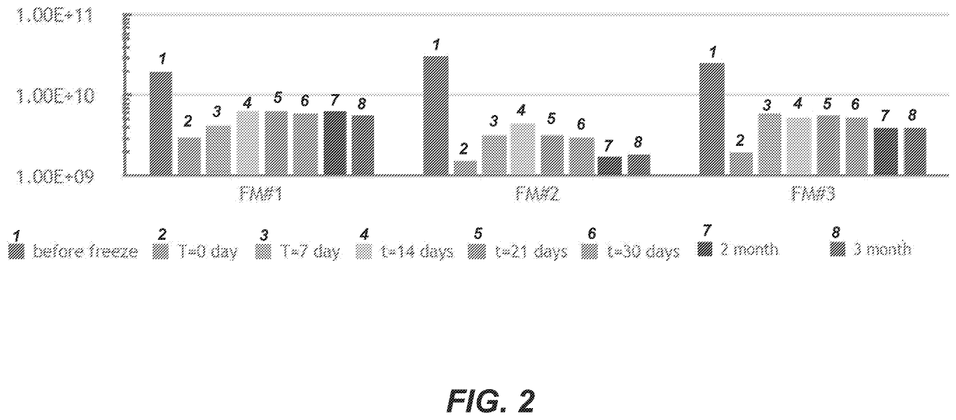

Initial cryoprotectant mixtures FM 1, 2, and 3 were evaluated in Eppendorf tubes stored at −20C after lyophilization for CFU recovery over 3 months with water addition. As shown in FIG. 1, FM1 and FM3 were identified as being superior (FIG. 2).

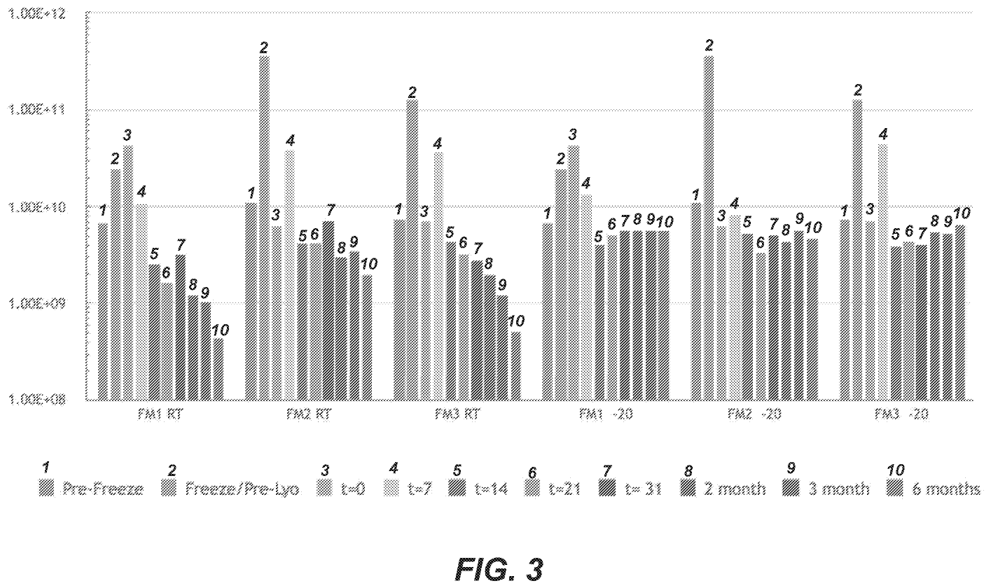

Subsequently, septum closure lyophilization-vials were produced using the 3 cryoprotectant mixtures and CFU recovery was evaluated over time at room temperature (17° C.) storage compared to −20° C. storage for non-milled lyophilized Sh-A9 “cakes” over 6 months. −20° C. storage demonstrated stable CFU recovery from all three cryoprotectant mixtures while 17° C. storage demonstrated 1 log reduction of CFU recovery by 6 months (FIG. 3).

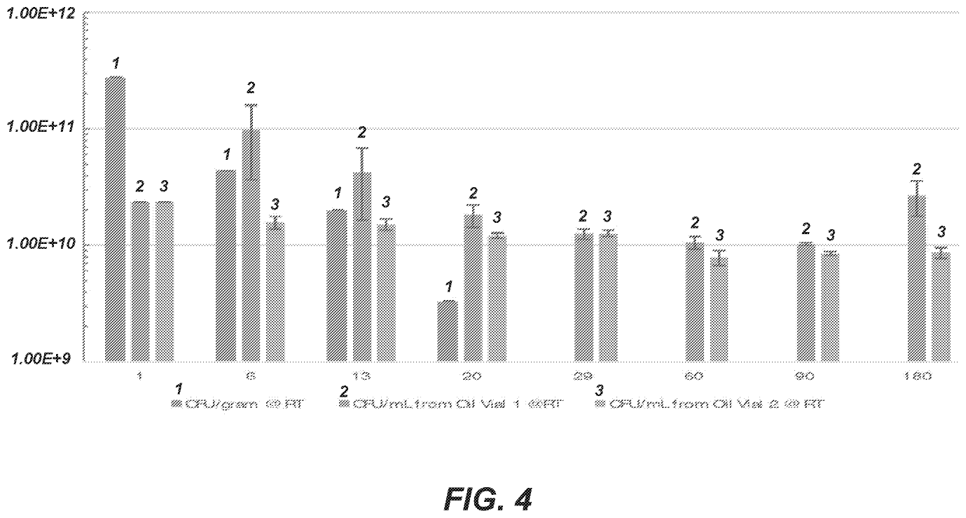

Bulk Sh-A9 cells were lyophilized in sterile culture flasks in the 3 cryoprotectant mixtures to evaluate the effect of dry milling/sieving of the “cracked bulk dried cakes” on stability of the resulting powered Sh-A9 Drug Substance and for use in initial anhydrous drug product formulations for stability assessment. Sh-A9 in FM#1 was sieved through a 100mesh screen and formulated in Sesame oil with fumed silica, then vialed and sealed under N₂ overlay and stored at 17° C. Triplicate sample from 2 vials at each time point were assayed for CFU recovery over 6 months. Powdered Sh-A9 stored at 17° C. and open to the atmosphere was also evaluated. As shown in FIG. 4, after the first 30 days stable recovery of CFUs from Sesame oil formulations stored at 17° C. is demonstrated and continues over the subsequent 5 months. Powdered Sh-A9 open to the atmosphere lost significant CFUs over the first 30 days and became “cakey” and difficult to handle and recovery evaluation was discontinued after 30 days.

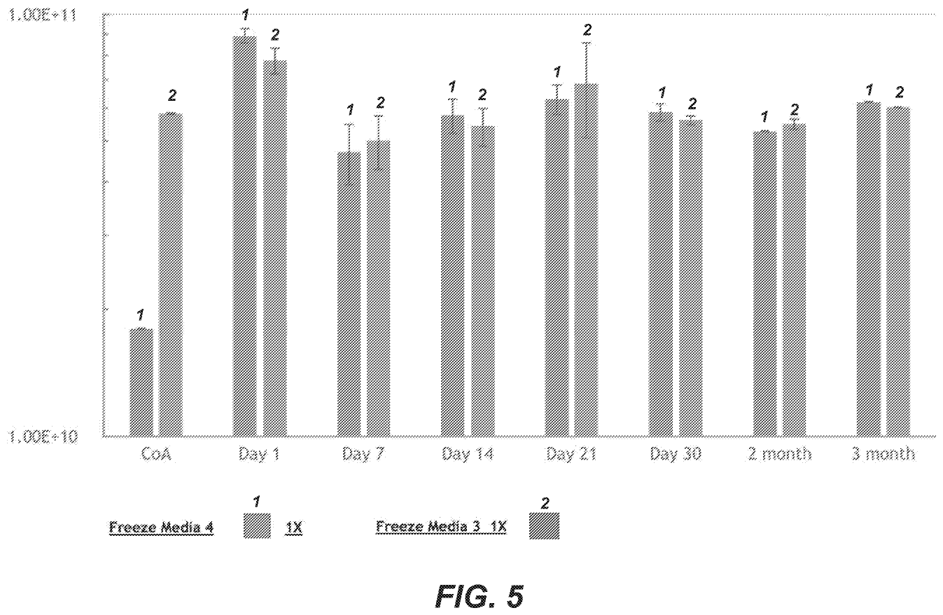

Additional lots of Sh-A9 lyophilized drug substance powder were produced representing Sh-A9 cell bulk lyophilized in FM#3 and FM#4. The freeze-dried bulk cakes were broken into pieces and milled through a 20mesh screen followed by a 60mesh screen. Powdered Sh-A9 was stored at −20° C. with desiccant. Dried powders were assayed for CFU recovery for stability. Six-month stability data of dried milled powders is presented in FIG. 5.

Through this process we were able to determine that Cryoprotectant FM#3, representing chemically defined, animal free and non-GMO ingredients is useful for drug substance stabilization. Further, Lyophilized Sh-A9 cells can be milled/sieved to at least 60 mesh and can be sieved to 100 mesh for use in anhydrous drug product formulations. Lyophilized Sh-A9 Drug Substance powder can be stored at −20° C. under desiccation, and 1 x 10¹¹ CFU/gram has been shown to be recoverable after storage.

### Example 8

### Development of Anhydrous Drug Product Using Stable Lyophilized Sh-A9 Drug Substance

For these studies, the following excipients were used:

Topical Oils (Croda Inc.-All Oils are Super Refined and compendia grade produced under cGMP): Sesame, Corn, Olive, Soy, Safflower. Soy, Corn and Sesame oils also supplied without BHT at 800 -1000 ppm added as antioxidant preservative for oils.

Other Topical Excipients (Croda Inc., Spectrum, Cabot Corp): Beeswax Super Refined (JP), Stearyl alcohol, Cetylstearyl alcohol, DL-tocopherol, CAB-O-Sil fumed silica M-5P

### Recovery of CFUs from Lyophilized Milled/Sieved Powders and Anti-SA Activity

Initial CFU counts were determined by adding small amount (eg: 20 to 40 mg) of milled/sieved Sh-A9 powder to a pre-weighed vial, reconstituting the powder in sterile water, and serially diluting into TSB and plating to determine colony growth. To determine CFU counts from anhydrous oil formulations, mixtures of milled/sieved Sh-A9 powder were mixed with various oils, and then extracted with sterile saline or AF-TSB, followed by serial dilution and plating as above. Sa113 UCSD was also plated on a top-agar lawn, and 0.003 mL diluted samples were spotted in order to determine zones of inhibition. Likewise, Sh-A9 FM#1 dry Powder was dusted on an Sa113 UCSD top-agar lawn to determine zones of inhibition for the dry product.

### Oil Formulation

0.15 grams of fumed silica (Cab-o-Sil M-5P, lot 3869248) was added to 50 mL Super Refined Sesame oil NF NP-LQ- (MH) [CRODA: batch 0001024665] followed by 2.0 grams of Sh-A9 FM#1 sieved powder. The bottle head space was N₂ purged, capped, and the contents vortexed to a homogeneous suspension. 2 mL of bacterial suspension was transferred to 4 mL sterile amber vials, N₂ was added to head space and vials capped. Bulk product bottle with bacterial suspension was re-vortexed after ever 2 vials were filled and N₂ sealed, to keep contents homogeneous. Sh-A9 FM#1 powder (0.18 gm) was transferred to a sterile vial for determination of the CFU gram in the powder. It was determined that the powder provided 2.77×10¹¹ cfu/gram. At various timepoints thereafter, samples were taken and assayed as above to determine the remaining CFU/ml in the formulation. It was expected that after room temperature incubation it would be possible to recover at least 1.11×10¹⁰ cfu/mL from the sample. To explore the effect of different batches and different oils, additional samples using different lots of Sh-A9 powder, designated CO-25-3A (FM#4) and CO-25-3B (FM#3) Sh-A9, were prepared using various oils as indicated below, with results as shown in table 2, below.

TABLE 2

| Grams powder | CO-25-3A (FM#4), | CO-25-3B (FM#3), |
|---|---|---|
| added to 50 mL oil | 1.80E+10 CFU/gram | 5.80E+10 CFU/gram |
| Soybean-USP | 0.23 | 0.27 |
| Soybean-USP-NP | 0.21 | 0.26 |
| Olive | 0.23 | 0.28 |
| Safflower-USP | 0.24 | 0.28 |

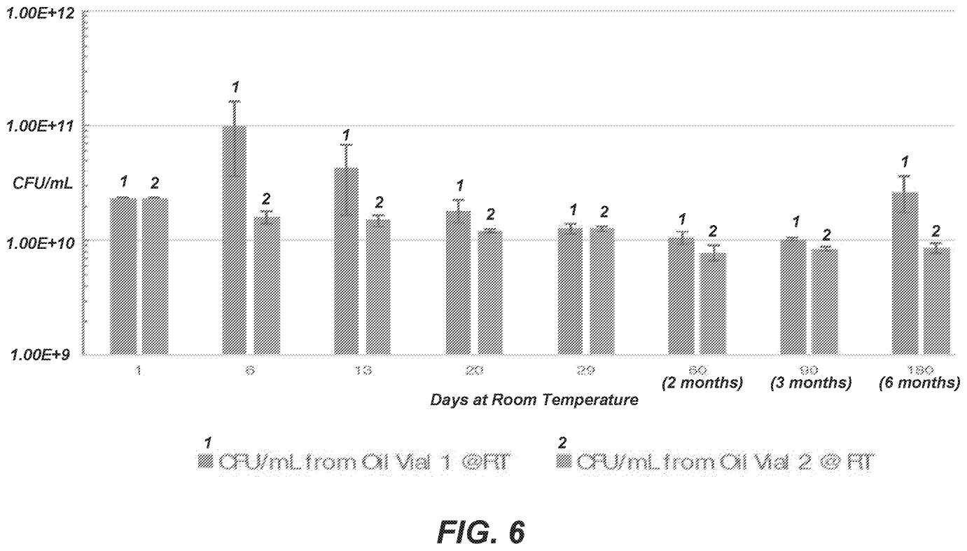

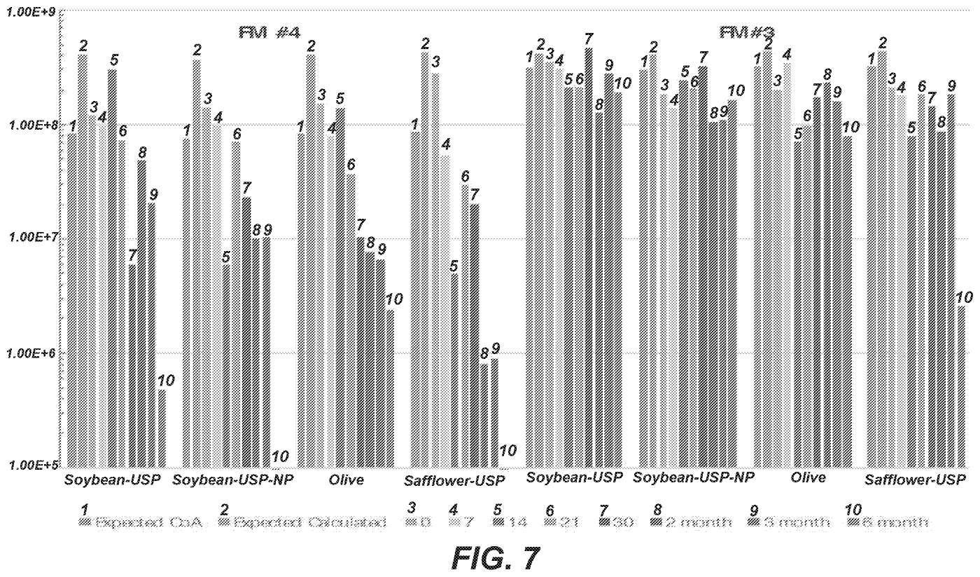

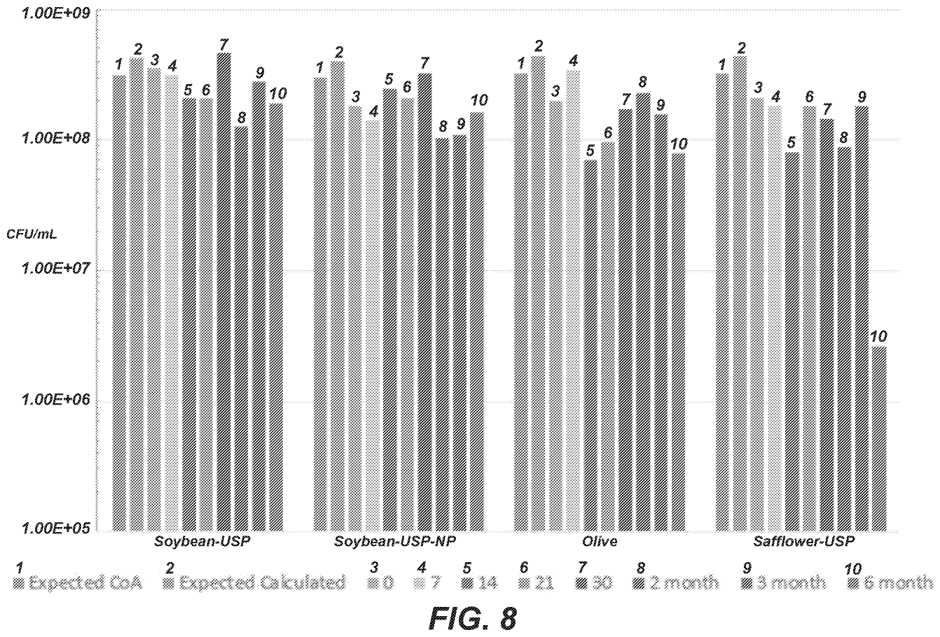

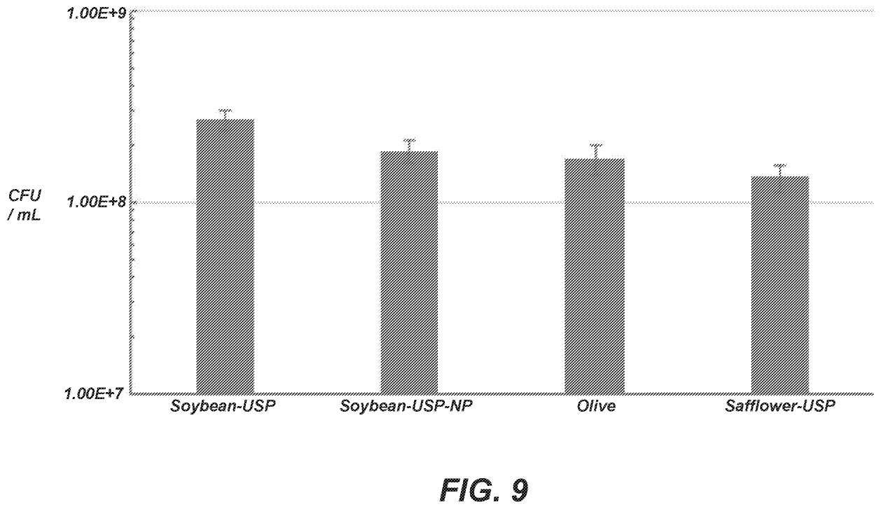

CFU recovery/stability determination from oil formulations at 6 months are shown in FIGS. 6-10.

Oil formulations containing FM#4 lyophilized powder formulations demonstrated decreases in recoverable CFUs within the first 30 days at 17° C. storage. FM#3 lyophilized powder oil formulations demonstrated stable recoverable CFUs for up to 6 months in Soy oil formulations. The presence of 800 to 1000 ppm BTH as an oil preservative had no effect on recoverable CFUs under the conditions tested. USP Soy oil with no preservative (NP) was used as a control to assess BHT toxicity to Sh-A9 Drug Products. USP Soy oil with or without BHT demonstrated similar CFU recovery over 6 months (FIG. 6).

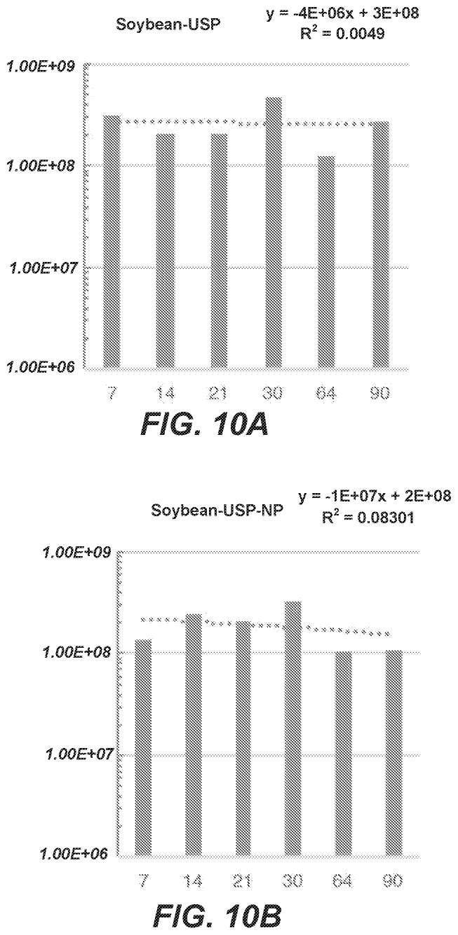

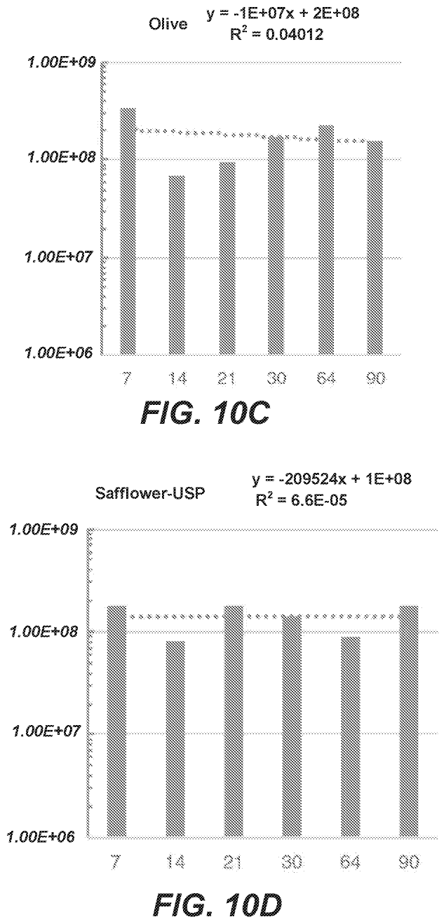

Expanded analysis using day 7 to day 90 CFU/mL recovery data (n=6 time points) suggests that the data variability measured may be due to the variability of the extraction process for any given sample rather than to product formulation destabilizing effects. Relative flatness of the regression trend lines and sigmoid point-to-point trend lines in the data support this interpretation (See FIGS. 10A-10D).

50% Cetaphil/50% Glycerol Fresh Formulation

A Sh-A9 colony was picked from a fresh agar plate seeded from a glycerol stock vial of UCSD MCB 20160628 and inoculated into 5 mL AF-TSB and grown overnight at 37° C. with 200 rpm shaking. The 5 mL Sh-A9 culture was seeded into a 500 mL production flask containing AF-TSB and grown overnight at 37° C. with 200 rpm shaking. To a 5 liter AF-TSB filled bioreactor at 37° C., 100 mL of the Sh-A9 flask culture was added and the oxygen sparged aerobic process initiated and OD600 was followed until ˜OD600 8 to 10 was reached to indicate culture harvest. A sample of harvested cells was plated by serial dilution to determine CFUs produced. The harvested culture was stored at 4° C. overnight prior to washing and product formulation.

Cells were harvested by centrifugation at 3000 g at 10° C. for 15 min in 500 mL bottles after overnight storage in conditioned medium at 4° C. Cell pellets were resuspended in 0.9% saline at 4° C. to wash cells. Cells were then pooled into a single bottle followed by centrifugation at 3000 g at 10° C. The cell pellet was washed twice with saline. 50% Cetaphil / 50% Glycerol Fresh Product Formulation was prepared as outlined in the ADRN TMT IND-008 and 0.4 mL stability aliquots were produced and stored under N₂ in amber vials at 4° C. or at 17° C.

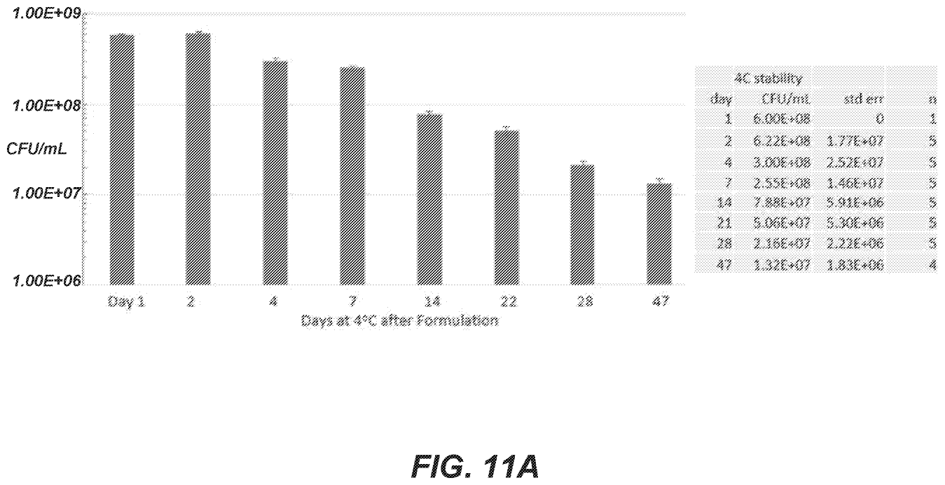

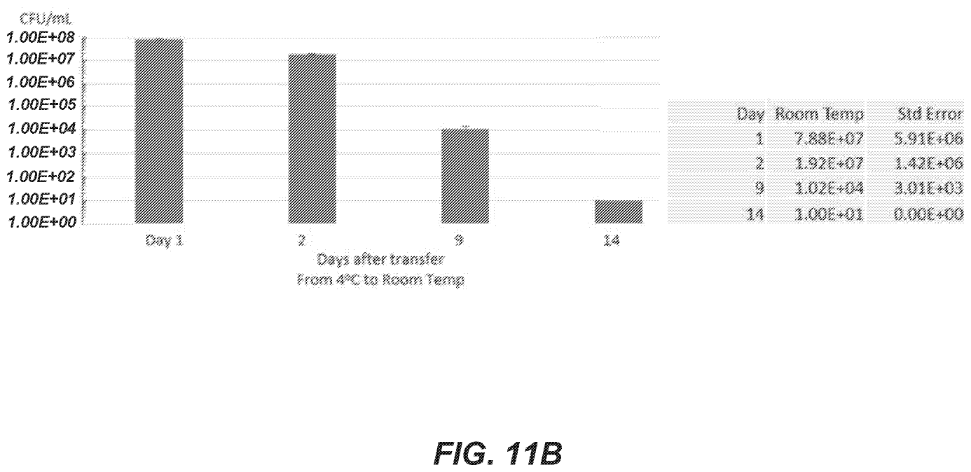

50% Cetaphil/50% Glycerol Fresh Product Formulation was stored at 17° C. and experience rapid multi-log reduction of recoverable CFUs, such that by day 9 of storage at 17° C. >3 logs of viable CFUs were lost (FIGS. 11A-11B)

50% Cetaphil/50% Glycerol Fresh Product Formulation stored at 4° C. experience a slower rate in the reduction of recoverable CFUs, and demonstrates at least a 30-day expiration date when stored at 4° C. Higher initial drug substance may be needed in these product formulations and additional development efforts for a cold chain product may be helpful. Evaluation of −20° C. long term storage with a 30 day expiration upon changing the temperature to 4° C. at the initiation of dosing could be considered. Fumed Silica Stabilization of Sh-A9 Oil Formulations and Accelerated Stability

Fumed silica (CAS number 112945-52-5) consists of microscopic droplets of amorphous silica fused into branched, chainlike, three-dimensional secondary particles that agglomerate into tertiary particles. The resulting powder has an extremely low bulk density and high surface area. Its three-dimensional structure results in viscosity-increasing, when used as a thickener or reinforcing filler. Fumed silica serves as a universal thickening agent and an anticaking agent (free-flow agent) with powders. Like silica gel, it serves as a desiccant to absorb water. It is used in cosmetics for its light-diffusing properties.

To maintain the anhydrous nature of the excipient oil in which lyophilized bacterial products are historically formulated, fumed silica has been added as a desiccant and for its anti-caking properties.

Early Sh-A9 oil formulation manufactured at the Drug Product CDMO were produced without the addition of fumed silica and CFU recovery from the product demonstrate high variability and lack of repeatability among replicates. Investigation into the CFU recovery issue identified that fumed silica was not added to the formulation, and that over time caking occurred and was visually identified in product sample vials. The product caking in vials affected the ability to homogeneously resuspend the lyophilized bacteria particles by vortex mixing and resulted in variability in CFUs recovered, irrespective of other destabilize processes occurring during storage.

To better understand the stabilizing properties of fumed silica in Sh-A9 anhydrous product formulations, the content of fumed silica to that of the Sh-A9 lyophilized powder in soy oil formulations was investigated.

To 50 mL bottles of super refined soy oil fumed silica was added, N₂ overlaid, capped and vortex and mix by inversion until a homogeneous suspension of fumed silica is observed. To each bottle, approximately 200 mg of CO-25-10 Sh-A9 Milled/Sieved Powder was added, N₂ overlaid, capped and vortex and mix by inversion until a homogeneous suspension was observed. Two mL aliquots were vialed under N₂ into sterile amber vials, capped and store at 17° C. in dark or 30° C. and 45° C. for accelerated temperature stability studies. CFU recovery/stability determination from oil formulation were performed as described above in Example 8. The ratio of fumed silica to drug substance for each formulation tested is shown in Table 3.

TABLE 3

| | mg fumed silica/mg |
|---|---|
| Formulation | Sh-A9 Drug Substance |
| 1 | 225 mg DS without fumed silica |
| 2 | 0.23 |
| 3 | 0.34 |
| 4 | 0.45 |
| 5 | 0.64 |

Resuspension of Sh-A9 formulated in soy oil and vialed without or with 0.23 mg fumed silica/mg DS demonstrated observable caking with product adherence to the container bottom upon product settling over time. Extensive vortex mixing of vials was not sufficient to suspend product into a homogeneous appearance. Different sized particles were apparent in vials upon attempted resuspension for assay time points. Large variations in CFUs recovered (log-fold) over short period of time at 17° C. were demonstrated and dependent on which vial was chosen and is consistent with non-homogeneous particle disruption and mixing due to caking. Samples with no or low silica to Sh-A9 powder ratios demonstrated lower variability in CFU recovered when stored at 30° C. and 45° C. compared to 17° C., but demonstrate greater total loss of recovered CFUs due to the accelerated thermal conditions (FIGS. 12A-12C).

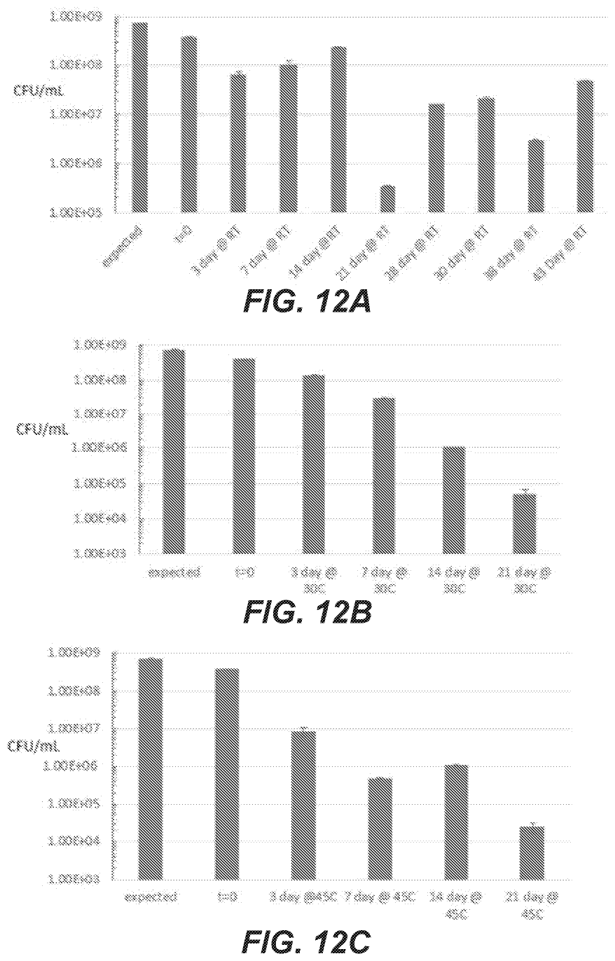

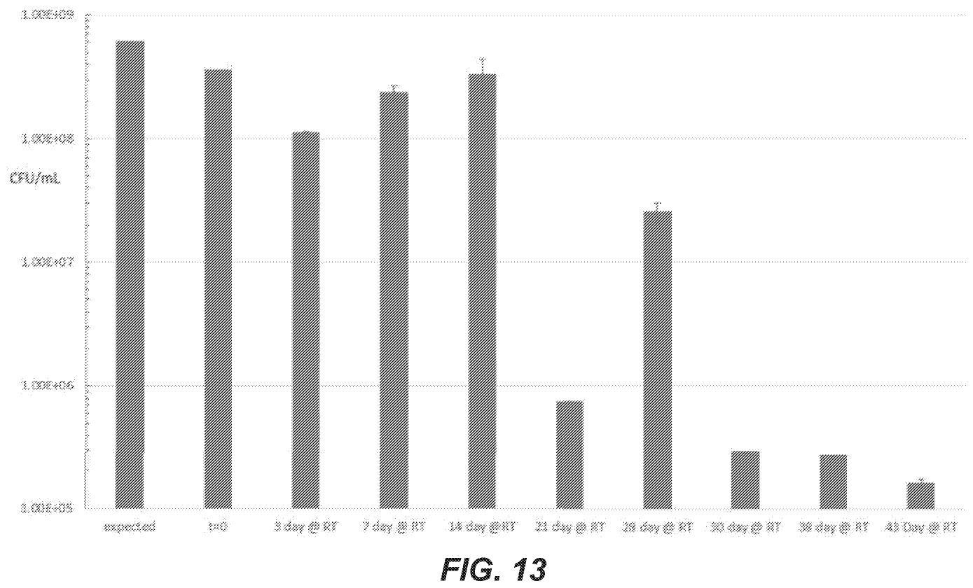

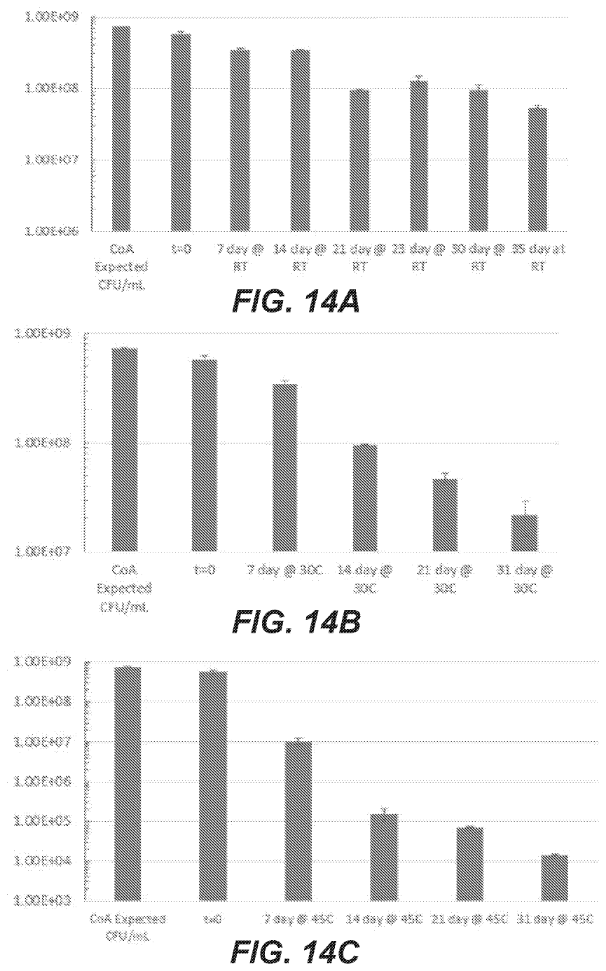

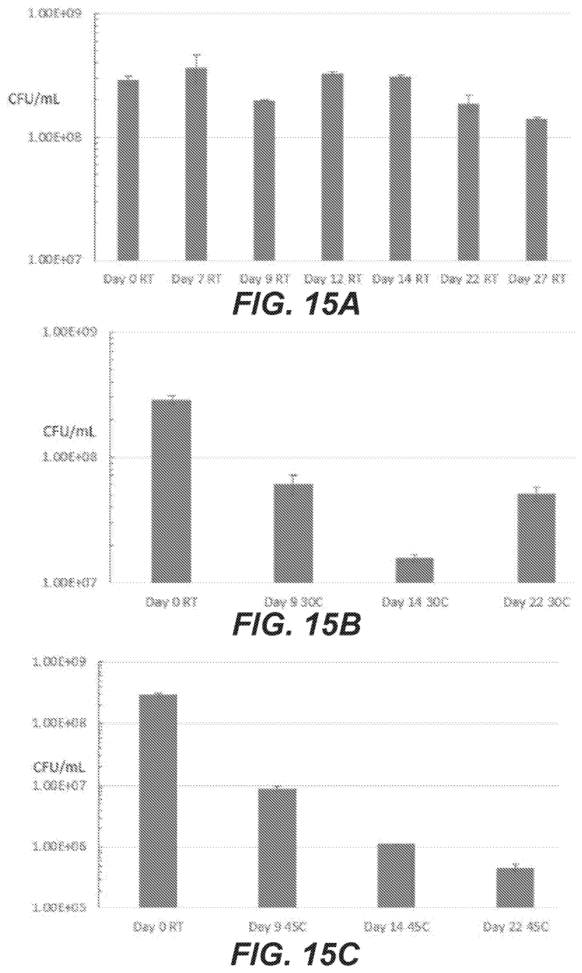

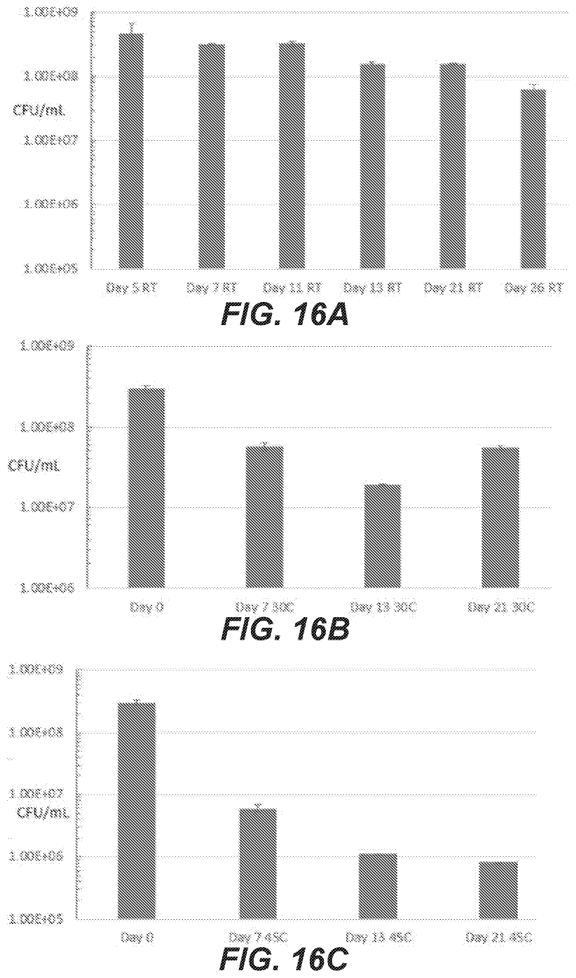

Increasing fumed silica to approximately 0.45 mg fumed silica per mg Sh-A9 lyophilized powder seems to increase recoverable CFUs from Soy oil formulations stored at 17° C. at early time points with consistent low variable results over time (FIGS. 12-16). Formulations with higher silica content produce homogeneous resuspensions of settled materials upon mild vortex or shaking. Caked product is not visible and product adherence to vial not detected under these conditions. Formulations with higher fumed silica content still demonstrate >1 log reduced CFU recovery within the first 14 days when stored at 30° C. and 45° C. compared to samples stored at 17° C. Increasing the fumed silica concentration to an amount at or above 0.45 mg/mg DS yields enhanced stability/CFU recovery over time under most temperature conditions tested (FIGS. 12-16).

### Anhydrous Lotion Formulations

1. Super Refined Bees Wax Thickened Oil Formulation

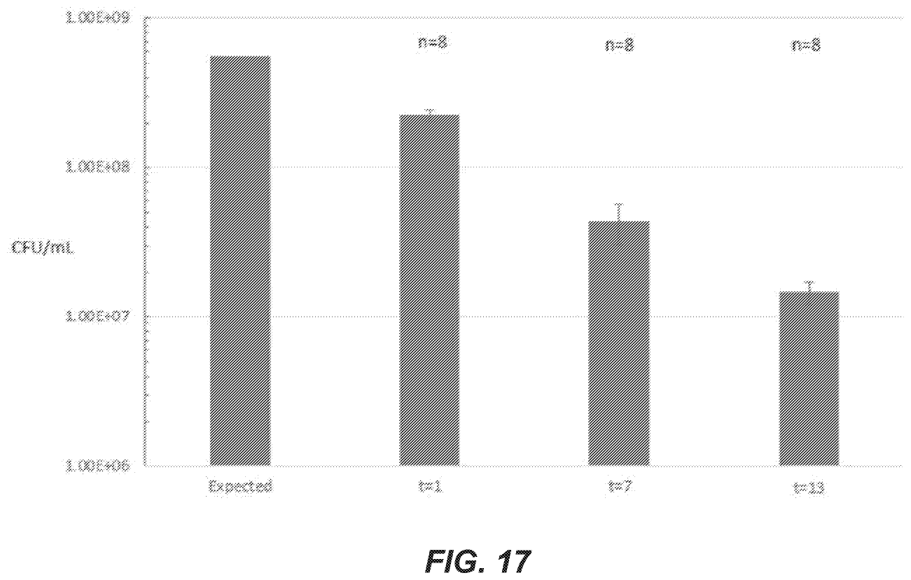

A thickened anhydrous formulation with a “cosmetically elegant feel” may appeal to patients better than a suspension formulated in oil. Initial investigations were performed with Super Refined Bees Wax (Croda) as a thickening agent and isopropyl palmitate (Croda) as a drying agent in a formulation containing 88% safflower oil, 5% beeswax, 5% isopropyl palmitate, 1% tocopherol, and 1% drug substance powder. As shown in FIG. 17, Sh-A9 lyophilized in FM#3 powder demonstrates reduced CFU recovery by >1 log when formulated in 5% Super refined bees wax as thickener in oil by day 13 post manufacture. Bees wax is naturally found in association with honey which has antimicrobial properties. Residual antimicrobial molecules may be present in Super Refined Bees Wax, and may be destabilizing or toxic to Sh-A9.

2. Fatty Alcohol Thickened Oil Formulations

Investigations into the use of alternative oil thickening excipients compared to the natural but chemically less defined bees wax have been initiated. Purified fatty alcohols represent a chemically defined and more stable alternative for use as a thickener in pharmaceutical formulations. The following formulations were prepared:

Lotion #1_formulation contains 0.34% drug substance powder, 85.18% soy oil, 6.81% stearyl alcohol, 6.81% cetostearyl alcohol, 0.85% vitamin E, and no fumed silica;

Lotion #2_formulation was prepared using Sh-A9 powder, homogenously suspended in fumed silica and soy oil (15% oil content) prior to addition to the remaining soy oil/fatty alcohol base lotion. The final formulation contained 0.42% Sh-A9 powder, 85% soy oil, 7% stearyl alcohol, 7% cetostearyl alcohol, 1% vitamin E, and 0.21% fumed silica.

Lotion Formulation #1 was dispensed into 3 sterile jars and stored at 17° C., 30° C., and 45° C. under N₂ atmosphere. Lotion Formulation #2 was dispensed into 2 sterile jars and stored at 17° C. and 30° C. under N₂ atmosphere.

Both formulations liquefied upon incubation at 45° C., thus Lotion #2 was dispensed for only 30° C. accelerated stability/recovery.

To determine CFU recovery from samples of the lotion formulations, samples of jar contents were mixed well and duplicate samples of lotion ranging from 200 mg to 400 mg were transferred to tared tubes to determine exact mass. Nine times the lotion mass transferred into tubes were calculated and that volume of AF-TSB was added to the lotion samples for CFU extraction by repeated vortex and shaking. Ten-fold serial dilution of the TSB phase containing extracted Sh-A9 cells were performed in AF-TSB to determine CFU recovery/stability.

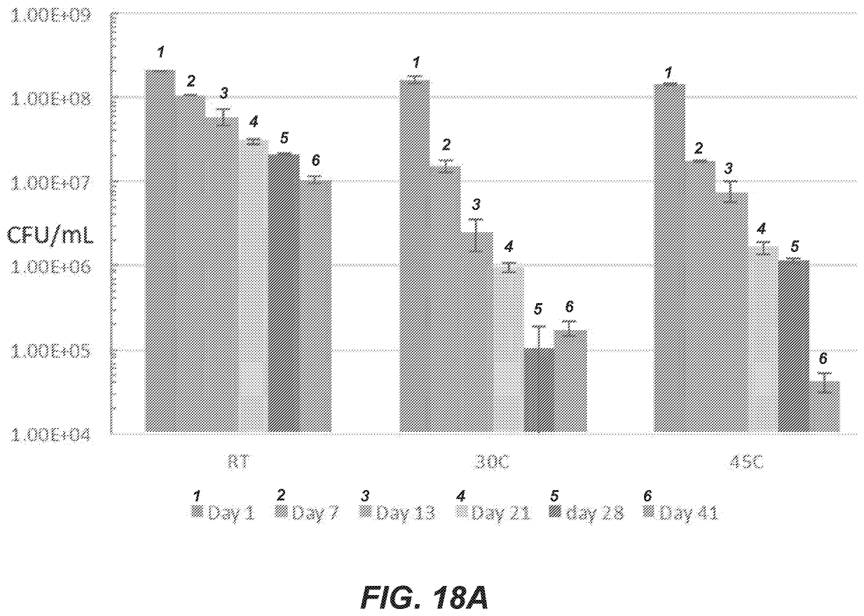

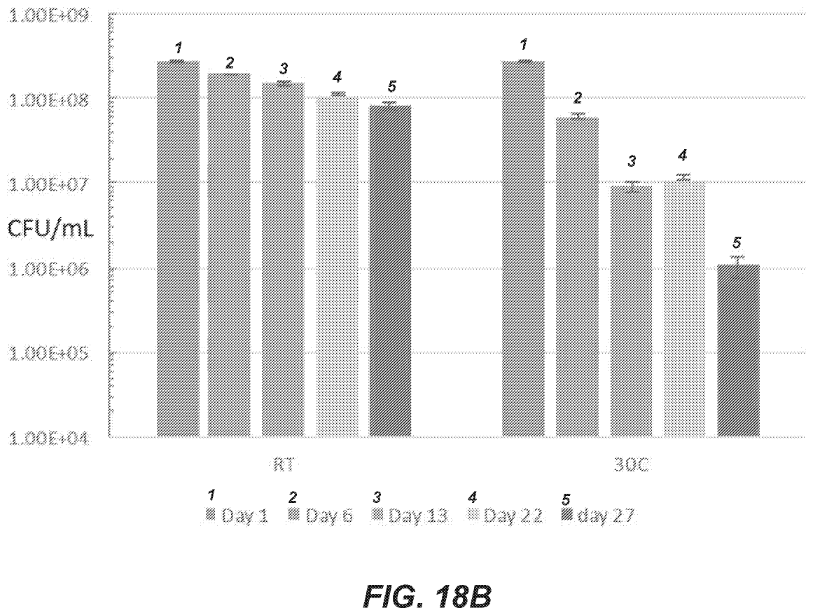

Accelerated Stability assessment of FM#3 Sh-A9 lyophilized power in soy oil and fatty alcohol based lotions display appreciable reduction in recoverable CFU at 30° C. within the first 13 days at accelerated temperatures (FIGS. 18A-18B). 45° C. storage liquefies the base lotion although recoverable CFUs seems to higher for 45° C. stored samples compared to that stored at 30° C. (FIGS. 18A-18B). Pre-mixing Sh-A9 lyophilized powder in soy oil and fumed silica to a homogeneous suspension prior to addition to the remainder of lotion base, is tending to increase recoverable CFU from lotions stored at 17° C. compared lotions prepared with fumed silica.

With respect to the use of substantially any plural and/or singular terms herein, those having skill in the art can translate from the plural to the singular and/or from the singular to plural as is appropriate to the context and/or application. The various singular/plural permutations can be expressly set forth herein for sake of clarity.

It will be understood by those within the art that, in general, terms used herein, and especially in the appended claims (for example, bodies of the appended claims) are generally intended as “open” terms (for example, the term “including” should be interpreted as “including but not limited to,” the term “having” should be interpreted as “having at least,” the term “includes” should be interpreted as “includes but is not limited to,” etc.). It will be further understood by those within the art that if a specific number of an introduced claim recitation is intended, such an intent will be explicitly recited in the claim, and in the absence of such recitation no such intent is present. For example, as an aid to understanding, the following appended claims can contain usage of the introductory phrases “at least one” and “one or more” to introduce claim recitations. However, the use of such phrases should not be construed to imply that the introduction of a claim recitation by the indefinite articles “a” or “an” limits any particular claim containing such introduced claim recitation to embodiments containing only one such recitation, even when the same claim includes the introductory phrases “one or more” or “at least one” and indefinite articles such as “a” or “an” (for example, “a” and/or “an” should be interpreted to mean “at least one” or “one or more”); the same holds true for the use of definite articles used to introduce claim recitations. In addition, even if a specific number of an introduced claim recitation is explicitly recited, those skilled in the art will recognize that such recitation should be interpreted to mean at least the recited number (for example, the bare recitation of “two recitations,” without other modifiers, means at least two recitations, or two or more recitations). Furthermore, in those instances where a convention analogous to “at least one of A, B, and C, etc.” is used, in general such a construction is intended in the sense one having skill in the art would understand the convention (for example, “ a system having at least one of A, B, and C” would include but not be limited to systems that have A alone, B alone, C alone, A and B together, A and C together, B and C together, and/or A, B, and C together, etc.). In those instances where a convention analogous to “at least one of A, B, or C, etc.” is used, in general such a construction is intended in the sense one having skill in the art would understand the convention (for example, “ a system having at least one of A, B, or C” would include but not be limited to systems that have A alone, B alone, C alone, A and B together, A and C together, B and C together, and/or A, B, and C together, etc.). It will be further understood by those within the art that virtually any disjunctive word and/or phrase presenting two or more alternative terms, whether in the description, claims, or drawings, should be understood to contemplate the possibilities of including one of the terms, either of the terms, or both terms. For example, the phrase “A or B” will be understood to include the possibilities of “A” or “B” or “A and B.”

In addition, where features or aspects of the disclosure are described in terms of Markush groups, those skilled in the art will recognize that the disclosure is also thereby described in terms of any individual member or subgroup of members of the Markush group.

As will be understood by one skilled in the art, for any and all purposes, such as in terms of providing a written description, all ranges disclosed herein also encompass any and all possible sub-ranges and combinations of sub-ranges thereof. Any listed range can be easily recognized as sufficiently describing and enabling the same range being broken down into at least equal halves, thirds, quarters, fifths, tenths, etc. As a non-limiting example, each range discussed herein can be readily broken down into a lower third, middle third and upper third, etc. As will also be understood by one skilled in the art all language such as “up to,” “at least,” “greater than,” “less than,” and the like include the number recited and refer to ranges which can be subsequently broken down into subranges as discussed above. Finally, as will be understood by one skilled in the art, a range includes each individual member. Thus, for example, a group having 1-3 articles refers to groups having 1, 2, or 3 articles. Similarly, a group having 1-5 articles refers to groups having 1, 2, 3, 4, or 5 articles, and so forth.

It will be further understood by one of ordinary skill in the art that the term “natural product” as used herein has its ordinary meaning in the art, and thus use lot this term does not constitute an admission or suggestion that the compositions disclosed herein constitute or are directed to a product of nature for purposes of analysis under 35 U.S.C. §101.

While various aspects and embodiments have been disclosed herein, other aspects and embodiments will be apparent to those skilled in the art. The various aspects and embodiments disclosed herein are for purposes of illustration and are not intended to be limiting, with the true scope and spirit being indicated by the following claims.

What is claimed is:

1. A composition, comprising one or more probiotic bacteria and further comprising an oil and at least one pharmaceutically acceptable excipient.

2. The composition of claim 1, wherein said one or more probiotic bacteria comprises a bacterium of the genus Staphylococcus.

3. The composition of any of claims 1 and 2, wherein said bacterium comprises one or more of Staphylococcus hominis and Staphylococcus epidermis.

4. The composition of any of claims 1-3, wherein said bacterium comprises a strain of Staphylococcus hominis.

5. The composition of any of claims 1-4, wherein said bacterium comprises Staphylococcus hominis strain A9.

6. The composition of any of claims 1-5, wherein said oil comprises one or more of soy oil, sesame oil, mineral oil, corn oil, olive oil, peanut oil, macadamia nut oil, canola oil, or emu oil.

7. The composition of any of claims 1-6, wherein said oil comprises soy oil.

8. The composition of any of claims 1-7, wherein said pharmaceutically acceptable excipient comprises one or more of tocopherol, monosodium glutamate, starch, colloidal silicon dioxide, microcrystalline cellulose, alginate, magnesium stearate, sodium stearate, stearyl alcohol, acetyl alcohol, cetostearyl alcohol, vinyl alcohol, or polyvinyl alcohol.

9. The composition of any of claims 1-8, wherein said composition is formulated as an oil composition.

10. The composition of any of claims 1-9, wherein said composition is formulated as a lotion composition.

11. The composition of any of claims 1-10, wherein said composition comprises an active drug substance or active cosmetic substance, wherein said active drug substance or active cosmetic substance comprises one or more probiotic bacteria, lyophilized bacteria, growth medium, lyophilized growth medium, bacterial extract, or lyophilized bacterial extract.

12. A composition, comprising:
an active drug substance comprising lyophilized coagulase negative Staphylococcus (CoNS);
an inert particle; and
an oil.

13. A composition, comprising:
an active drug substance comprising lyophilized Staphylococcus hominis strain A9;
fumed silica; and
an oil selected from the group comprising soy oil, sesame oil, mineral oil, corn oil, olive oil, peanut oil, macadamia nut oil, canola oil, emu oil, or any combination thereof.

14. The composition of any one of claims 1-13, wherein the composition comprises a sugar, a starch, a cellulose, powdered tragacanth, malt, gelatin, talc, a solid lubricant, calcium sulfate, a vegetable oil, a polyol, alginic acid; a TWEEN, sodium lauryl sulfate, an emulsifier, a wetting agent, a coloring agent, a flavoring agent, a tableting agent, a stabilizer; an antioxidant, a preservative, pyrogen-free water, isotonic saline, a phosphate buffer solution, or any combination thereof.

15. The composition of any of claims 1-14, wherein said active drug substance is present as a particle, microparticle, or nanoparticle.

16. The composition of claim 12, wherein said particle, microparticle, or nanoparticle has an average diameter of between 1 nm and 1 mm, between 10 nm and 500 um, between 100 nm and 100 um, between 1 um and 250 um, between 10 um and 100 um, between 10 um and 50 um, or between 20 um and 30 um.

17. The composition of any of claims 1-16, wherein said composition is stable for 2 months or more at room temperature, stable for 4 months or more at room temperature, or stable for 6 months or more at room temperature.

18. The composition of any of claims 1-17, wherein said composition retains at least 50%, at least 70%, or at least 80% of its colony forming activity for 2 months or more at room temperature at 4° C.

19. The composition of any of claims 1-18, wherein said composition is stable for 2 months or more, for 4 months or more, for 6 months or more, at 4° C.

20. The composition of any of claims 1-19, wherein said composition is anhydrous.

21. The composition of any of claims 1-20, wherein said composition is substantially free of water or moisture.

22. A method of treating a disease, disorder, or condition of the skin by administering to a subject in need thereof a composition according to any of claims 1-21.

23. The method of claim 22, wherein said disease, disorder, or condition comprises one or more of atopic dermatitis, eczema, pyotraumatic dermatitis, pyoderma, superficial pyoderma, folliculitis, rosacea, Netherton syndrome, acne, wounds (including abrasions, radiation damage, and burns), psoriasis, mastitis, icthyosis, lichen formation, and sebhorreic dermatitis, or any combination thereof.

24. The method of any of claims 22-23, wherein said disease, disorder, or condition comprises a bacterial infection, overgrowth, or dysbiosis.

25. The method of any of claims 22-24, wherein said bacterial infection, overgrowth, or dysbiosis comprises colonization, overgrowth, or infection with one or more of Staphylococcus aureus, Staphylococcus intermedius, Staphylococcus pseudintermedius, Staphylococcus felis, Staphylococcus schleiferi, Micrococcus spp., Acinetobacter spp., and/or alpha hemolytic streptococci, Cutibacterium acnes or any combination thereof.
